SEQUENCE LISTING (160 sequences)

SEQ ID NO: 1 - RNA, 401 nt, Artificial Sequence
  Synthetic Sequence
cuggaccucccaaaagccaacuuauugugauauuuguaaauuauaguuuuagcaguucguuugccacaugaguggaacaucgugaaugcacuuuugauaagugcucgguuauuuuauauuguaacuaccagccuucagaggcgaucguaugcauaguuucuugaagucaauuuguccguguauucaaauguuugcuuucgugaaaacucgcauuguuuugucacucuaccaaguaaucaauuuguaccaaucaaucgcauaugguuguccuagaucuaaaaauggcaauaauuugccuucgguauugcaccuaauguauucaagaacaaguagggaagcucgaaauuucucaaauacuuacccaaaaaauagauagaaauauauuuucgauucgcaaucgu

SEQ ID NO: 2 - DNA, 40 bp, Artificial Sequence
  Synthetic sequence
aagctcgaaatttctcaaatacttacccaaaaaatagata

SEQ ID NO: 3 - DNA, 60 bp, Artificial Sequence
  Synthetic Sequence
accgttgtagcaatacttctttgattagtaataacatcactatctattttttgggtaagt

SEQ ID NO: 4 - DNA, 60 bp, Artificial Sequence
  Synthetic Sequence
atttgagaaatttcgagctttcaaccgattgagggagggaaggtaaatattgacggaaat

SEQ ID NO: 5 - DNA, 55 bp, Artificial Sequence
  Synthetic Sequence
tgtagcaatacttctttgattagtaataacatcacattttttgggtaagtcagtc

SEQ ID NO: 6 - DNA, 55 bp, Artificial Sequence
  Synthetic Sequence
gtcagatttgagaaatttcgtcaaccgattgagggagggaaggtaaatattgacg

SEQ ID NO: 7 - DNA, 52 bp, Artificial Sequence
  Synthetic Sequence
tgtagcaatacttctttgattagtaataacatcacttttgggtaagtcagtc

SEQ ID NO: 8 - DNA, 52 bp, Artificial Sequence
  Synthteic Sequence
gtcagatttgagaaatttcaaccgattgagggagggaaggtaaatattgacg

SEQ ID NO: 9 - DNA, 48 bp, Artificial Sequence
  Synthetic Sequence
tgtagcaatacttctttgattagtaataacatcacgggtaagtcagtc

SEQ ID NO: 10 - DNA, 48 bp, Artificial Sequence
  Synthetic Sequence
gtcagatttgagatcaaccgattgagggagggaaggtaaatattgacg

SEQ ID NO: 11 - DNA, 60 bp, Artificial Sequence
  Synthetic Sequence
atttgagaaatttcgagctttgggttatataactatatgtaaatgctgatgcaaatccaa

SEQ ID NO: 12 - RNA, 22 nt, Artificial Sequence
  Synthtetic sequence
uggaauguaaggaagugugugg

SEQ ID NO: 13 - DNA, 22 bp, Artificial Sequence
  Synthetic sequence
tggaatgtaaggaagtgtgtgg

SEQ ID NO: 14 - DNA, 51 bp, Artificial Sequence
  Synthetic Sequence
accgttgtagcaatacttctttgattagtaataacatcacccacacacttc

SEQ ID NO: 15 - DNA, 51 bp, Artificial Sequence
  Synthetic Sequence
cttacattccatcaaccgattgagggagggaaggtaaatattgacggaaat

SEQ ID NO: 16 - RNA, 157 nt, Artificial Sequence
  Synthetic Sequence
cgacucuuagcgguggaucacucggcucgugcgucgaugaagaacgcagcuagcugcgagaauuaaugugaauugcaggacacauugaucaucgacacuucgaacgcacuugcggccccggguuccucccggggcuacgccugucugagcgucgcuu

SEQ ID NO: 17 - RNA, 22 nt, Artificial Sequence
  Synthetic Sequence
uggaauguaaggaagugugugg

SEQ ID NO: 18 - DNA, 55 bp, Artificial Sequence
  Synthetic Sequence
accgttgtagcaatacttctttgattagtaataacatcactcatcgacgcacgag

SEQ ID NO: 19 - DNA, 55 bp, Artificial Sequence
  Synthetic Sequence
ccgagtgatccaccgtcaaccgattgagggagggaaggtaaatattgacggaaat

SEQ ID NO: 20 - RNA, 121 nt, Artificial Sequence
  Synthetic Sequence
gucuacggccauaccacccugaacgcgcccgaucucgucugaucucggaagcuaagcagggucgggccugguuaguacuuggaugggagaccgccugggaauaccgggugcuguaggcuuu

SEQ ID NO: 21 - DNA, 30 bp, Artificial Sequence
  Synthetic Sequence
tagtacttggatgggagaccgcctgggaat

SEQ ID NO: 22 - DNA, 55 bp, Artificial Sequence
  Synthetic Sequence
accgttgtagcaatacttctttgattagtaataacatcacattcccaggcggtct

SEQ ID NO: 23 - DNA, 55 bp, Artificial Sequence
  Synthetic Sequence
cccatccaagtactatgggttatataactatatgtaaatgctgatgcaaatccaa

SEQ ID NO: 24 - RNA, 31 nt, Artificial Sequence
  Synthetic Sequence
uagucugcaccugcaccagucgcucagggau

SEQ ID NO: 25 - DNA, 31 bp, Artificial Sequence
  Synthetic Sequence
tagtctgcacctgcaccagtcgctcagggat

SEQ ID NO: 26 - DNA, 50 bp, Artificial Sequence
  Synthetic Sequence
accgttgtagcaatacttctttgattagtaataacatcacatccctgagc

SEQ ID NO: 27 - DNA, 50 bp, Artificial Sequence
  Synthetic sequence
gactggtgcatcaaccgattgagggagggaaggtaaatattgacggaaat

SEQ ID NO: 28 - DNA, 55 bp, Artificial Sequence
  Synthetic Sequence
accgttgtagcaatacttctttgattagtaataacatcactccctgagcgactgg

SEQ ID NO: 29 - DNA, 55 bp, Artificial Sequence
  Synthetic sequence
tgcaggtgcagactatcaaccgattgagggagggaaggtaaatattgacggaaat

SEQ ID NO: 30 - DNA, 60 bp, Artificial Sequence
  Synthetic sequence
agagcataaagctaaatcggttgtaccaaaaacattatgaccctgtaatacttttgcggg

SEQ ID NO: 31 - DNA, 60 bp, Artificial Sequence
  Synthetic sequence
agaagcctttatttcaacgcaaggataaaaatttttagaaccctcatatattttaaatgc

SEQ ID NO: 32 - DNA, 60 bp, Artificial Sequence
  Synthetic sequence
aatgcctgagtaatgtgtaggtaaagattcaaaagggtgagaaaggccggagacagtcaa

SEQ ID NO: 33 - DNA, 60 bp, Artificial Sequence
  Synthetic Sequence
atcaccatcaatatgatattcaaccgttctagctgataaattaatgccggagagggtagc

SEQ ID NO: 34 - DNA, 60 bp, Artificial Sequence
  Synthetic sequence
tatttttgagagatctacaaaggctatcaggtcattgcctgagagtctggagcaaacaag

SEQ ID NO: 35 - DNA, 60 bp, Artificial Sequence
  Synthetic Sequence
agaatcgatgaacggtaatcgtaaaactagcatgtcaatcatatgtaccccggttgataa

SEQ ID NO: 36 - DNA, 60 bp, Artificial Sequence
  Synthetic Sequence
tcagaaaagccccaaaaacaggaagattgtataagcaaatatttaaattgtaaacgttaa

SEQ ID NO: 37 - DNA, 60 bp, Artificial Sequence
  Synthetic Sequence
tattttgttaaaattcgcattaaatttttgttaaatcagctcattttttaaccaatagga

SEQ ID NO: 38 - DNA, 60 bp, Artificial Sequence
  Synthetic sequence
acgccatcaaaaataattcgcgtctggccttcctgtagccagctttcatcaacattaaat

SEQ ID NO: 39 - DNA, 60 bp, Artificial Sequence
  synthetic sequence
ggataggtcacgttggtgtagatgggcgcatcgtaaccgtgcatctgccagtttgagggg

SEQ ID NO: 40 - DNA, 60 bp, Artificial Sequence
  Synthetic Sequence
acgacgacagtatcggcctcaggaagatcgcactccagccagctttccggcaccgcttct

SEQ ID NO: 41 - DNA, 60 bp, artificial sequence
  Synthetic sequence
ggtgccggaaaccaggcaaagcgccattcgccattcaggctgcgcaactgttgggaaggg

SEQ ID NO: 42 - DNA, 60 bp, Artificial sequence
  Synthetic sequence
cgatcggtgcgggcctcttcgctattacgccagctggcgaaagggggatgtgctgcaagg

SEQ ID NO: 43 - DNA, 60 bp, Artificial Sequence
  Synthetic sequence
cgattaagttgggtaacgccagggttttcccagtcacgacgttgtaaaacgacggccagt

SEQ ID NO: 44 - DNA, 60 bp, Artificial Sequence
  Synthetic sequence
gccaagcttgcatgcctgcaggtcgactctagaggatccccgggtaccgagctcgaattc

SEQ ID NO: 45 - DNA, 60 bp, artificial sequence
  synthetic sequences
gtaatcatggtcatagctgtttcctgtgtgaaattgttatccgctcacaattccacacaa

SEQ ID NO: 46 - DNA, 60 bp, Artificial Sequence
  Synthetic sequence
catacgagccggaagcataaagtgtaaagcctggggtgcctaatgagtgagctaactcac

SEQ ID NO: 47 - DNA, 60 bp, Artificial Sequence
  synthetic sequence
attaattgcgttgcgctcactgcccgctttccagtcgggaaacctgtcgtgccagctgca

SEQ ID NO: 48 - DNA, 60 bp, Artificial Sequence
  synthetic sequence
ttaatgaatcggccaacgcgcggggagaggcggtttgcgtattgggcgccagggtggttt

SEQ ID NO: 49 - DNA, 60 bp, Artificial Sequence
  synthetic sequence
gttgcagcaagcggtccacgctggtttgccccagcaggcgaaaatcctgtttgatggtgg

SEQ ID NO: 50 - DNA, 60 bp, Artificial Sequence
  synthetic sequence
ttccgaaatcggcaaaatcccttataaatcaaaagaatagcccgagatagggttgagtgt

SEQ ID NO: 51 - DNA, 60 bp, Artificial Sequence
  synthetic sequence
tgttccagtttggaacaagagtccactattaaagaacgtggactccaacgtcaaagggcg

SEQ ID NO: 52 - DNA, 60 bp, Artificial Sequence
  synthetic sequence
aaaaaccgtctatcagggcgatggcccactacgtgaaccatcacccaaatcaagtttttt

SEQ ID NO: 53 - DNA, 60 bp, Artificial Sequence
  synthetic sequence
ggggtcgaggtgccgtaaagcactaaatcggaaccctaaagggagcccccgatttagagc

SEQ ID NO: 54 - DNA, 60 bp, Artificial Sequence
  synthetic sequence
ttgacggggaaagccggcgaacgtggcgagaaaggaagggaagaaagcgaaaggagcggg

SEQ ID NO: 55 - DNA, 60 bp, Artificial Sequence
  synthetic sequence
cgctagggcgctggcaagtgtagcggtcacgctgcgcgtaaccaccacacccgccgcgct

SEQ ID NO: 56 - DNA, 60 bp, Artificial Sequence
  synthetic sequence
taatgcgccgctacagggcgcgtactatggttgctttgacgagcacgtataacgtgcttt

SEQ ID NO: 57 - DNA, 60 bp, Artificial sequence
  synthetic sequence
cctcgttagaatcagagcgggagctaaacaggaggccgattaaagggattttagacagga

SEQ ID NO: 58 - DNA, 60 bp, Artificial sequence
  synthetic sequence
acggtacgccagaatcctgagaagtgtttttataatcagtgaggccaccgagtaaaagag

SEQ ID NO: 59 - DNA, 60 bp, Artificial Sequence
  synthetic sequence
ttgcctgagtagaagaactcaaactatcggccttgctggtaatatccagaacaatattac

SEQ ID NO: 60 - DNA, 60 bp, Artificial Sequence
  synthetic sequence
cgccagccattgcaacaggaaaaacgctcatggaaatacctacattttgacgctcaatcg

SEQ ID NO: 61 - DNA, 60 bp, Artificial Sequence
  synthetic sequence
tctgaaatggattatttacattggcagattcaccagtcacacgaccagtaataaaaggga

SEQ ID NO: 62 - DNA, 60 bp, Artificial sequence
  synthetic sequence
cattctggccaacagagatagaacccttctgacctgaaagcgtaagaatacgtggcacag

SEQ ID NO: 63 - DNA, 60 bp, Artificial Sequence
  synthetic sequence
acaatatttttgaatggctattagtctttaatgcgcgaactgatagccctaaaacatcgc

SEQ ID NO: 64 - DNA, 60 bp, Artificial Sequence
  synthetic sequence
cattaaaaataccgaacgaaccaccagcagaagataaaacagaggtgaggcggtcagtat

SEQ ID NO: 65 - DNA, 60 bp, Artificial Sequence
  synthetic sequence
taacaccgcctgcaacagtgccacgctgagagccagcagcaaatgaaaaatctaaagcat

SEQ ID NO: 66 - DNA, 60 bp, Artificial Sequence
  synthetic sequence
caccttgctgaacctcaaatatcaaaccctcaatcaatatctggtcagttggcaaatcaa

SEQ ID NO: 67 - DNA, 60 bp, Artificial Sequence
  synthetic sequence
cagttgaaaggaattgaggaaggttatctaaaatatctttaggagcactaacaactaata

SEQ ID NO: 68 - DNA, 60 bp, Artificial Sequence
  synthetic sequence
gattagagccgtcaatagataatacatttgaggatttagaagtattagactttacaaaca

SEQ ID NO: 69 - DNA, 60 bp, Artificial Sequence
  synthetic sequence
cattatcattttgcggaacaaagaaaccaccagaaggagcggaattatcatcatattcct

SEQ ID NO: 70 - DNA, 60 bp, Artificial Sequence
  synthetic sequence
gattatcagatgatggcaattcatcaatataatcctgattgtttggattatacttctgaa

SEQ ID NO: 71 - DNA, 60 bp, Artificial Sequence
  synthetic sequence
taatggaagggttagaacctaccatatcaaaattatttgcacgtaaaacagaaataaaga

SEQ ID NO: 72 - DNA, 60 bp, Artificial Sequence; identical to SEQ ID NO: 71
  synthetic sequence

SEQ ID NO: 73 - DNA, 60 bp, Artificial Sequence
  synthetic sequence
cgggagaaacaataacggattcgcctgattgctttgaataccaagttacaaaatcgcgca

SEQ ID NO: 74 - DNA, 60 bp, Artificial Sequence
  synthetic sequence
gaggcgaattattcatttcaattacctgagcaaaagaagatgatgaaacaaacatcaaga

SEQ ID NO: 75 - DNA, 60 bp, Artificial Sequence
  synthetic sequence
aaacaaaattaattacatttaacaatttcatttgaattaccttttttaatggaaacagta

SEQ ID NO: 76 - DNA, 60 bp, Artificial Sequence
  synthetic sequence
cataaatcaatatatgtgagtgaataaccttgcttctgtaaatcgtcgctattaattaat

SEQ ID NO: 77 - DNA, 60 bp, Artificial Sequence
  synthetic sequence
tttcccttagaatccttgaaaacatagcgatagcttagattaagacgctgagaagagtca

SEQ ID NO: 78 - DNA, 60 bp, Artificial Sequences
  synthetic sequences
atagtgaatttatcaaaatcataggtctgagagactacctttttaacctccggcttaggt

SEQ ID NO: 79 - DNA, 60 bp, Artificial Sequence
  synthetic sequence
gaaaactttttcaaatatattttagttaatttcatcttctgacctaaatttaatggtttg

SEQ ID NO: 80 - DNA, 60 bp, Artificial Sequence
  synthetic sequence
aaataccgaccgtgtgataaataaggcgttaaataagaataaacaccggaatcataatta

SEQ ID NO: 81 - DNA, 60 bp, Artificial Sequence
  synthetic sequence
ctagaaaaagcctgtttagtatcatatgcgttatacaaattcttaccagtataaagccaa

SEQ ID NO: 82 - DNA, 60 bp, Artificial Sequence
  synthetic sequence
cgctcaacagtagggcttaattgagaatcgccatatttaacaacgccaacatgtaattta

SEQ ID NO: 83 - DNA, 60 bp, Artificial Sequence
  synthetic sequence
ggcagaggcattttcgagccagtaataagagaatataaagtaccgacaaaaggtaaagta

SEQ ID NO: 84 - DNA, 60 bp, Artificial Sequence
  synthetic sequence
attctgtccagacgacgacaataaacaacatgttcagctaatgcagaacgcgcctgttta

SEQ ID NO: 85 - DNA, 60 bp, Artificial Sequence
  synthetic sequence
tcaacaatagataagtcctgaacaagaaaaataatatcccatcctaatttacgagcatgt

SEQ ID NO: 86 - DNA, 60 bp, Artificial Sequence
  synthetic sequence
agaaaccaatcaataatcggctgtctttccttatcattccaagaacgggtattaaaccaa

SEQ ID NO: 87 - DNA, 60 bp, Artificial Sequence
  synthetic sequence
gtaccgcactcatcgagaacaagcaagccgtttttattttcatcgtaggaatcattaccg

SEQ ID NO: 88 - DNA, 60 bp, Artificial Sequence
  synthetic sequence
cgcccaatagcaagcaaatcagatatagaaggcttatccggtattctaagaacgcgaggc

SEQ ID NO: 89 - DNA, 60 bp, Artificial Sequence
  synthetic sequence
attttgcacccagctacaattttatcctgaatcttaccaacgctaacgagcgtctttcca

SEQ ID NO: 90 - DNA, 60 bp, Artificial Sequence
  synthetic sequence
gagcctaatttgccagttacaaaataaacagccatattatttatcccaatccaaataaga

SEQ ID NO: 91 - DNA, 60 bp, Artificial Sequence
  synthetic sequence
aacgattttttgtttaacgtcaaaaatgaaaatagcagcctttacagagagaataacata

SEQ ID NO: 92 - DNA, 60 bp, Artificial Sequence
  synthetic sequence
aaaacagggaagcgcattagacgggagaattaactgaacaccctgaacaaagtcagaggg

SEQ ID NO: 93 - DNA, 60 bp, Artificial Sequence
  synthetic sequence
taattgagcgctaatatcagagagataacccacaagaattgagttaagcccaataataag

SEQ ID NO: 94 - DNA, 60 bp, Artificial Sequence
  synthetic sequence
agcaagaaacaatgaaatagcaatagctatcttaccgaagccctttttaagaaaagtaag

SEQ ID NO: 95 - DNA, 60 bp, Artificial Sequence
  synthetic sequence
cagatagccgaacaaagttaccagaaggaaaccgaggaaacgcaataataacggaatacc

SEQ ID NO: 96 - DNA, 60 bp, Artificial Sequence
  synthetic sequence
caaaagaactggcatgattaagactccttattacgcagtatgttagcaaacgtagaaaat

SEQ ID NO: 97 - DNA, 60 bp, Artificial Sequence
  synthetic sequence
acatacataaaggtggcaacatataaaagaaacgcaaagacaccacggaataagtttatt

SEQ ID NO: 98 - DNA, 60 bp, Artificial sequence
  synthetic sequence
ttgtcacaatcaatagaaaattcatatggtttaccagcgccaaagacaaaagggcgacat

SEQ ID NO: 99 - DNA, 60 bp, Artificial Sequence
  synthetic sequence
tcaccgtcaccgacttgagccatttgggaattagagccagcaaaatcaccagtagcacca

SEQ ID NO: 100 - DNA, 60 bp, Artificial Sequence
  synthetic sequence
ttaccattagcaaggccggaaacgtcaccaatgaaaccatcgatagcagcaccgtaatca

SEQ ID NO: 101 - DNA, 60 bp, Artificial sequence
  synthetic sequence
gtagcgacagaatcaagtttgcctttagcgtcagactgtagcgcgttttcatcggcattt

SEQ ID NO: 102 - DNA, 60 bp, Artificial Sequence
  synthetic sequence
tcggtcatagcccccttattagcgtttgccatcttttcataatcaaaatcaccggaacca

SEQ ID NO: 103 - DNA, 60 bp, Artificial Sequence
  synthetic sequence
gagccaccaccggaaccgcctccctcagagccgccaccctcagaaccgccaccctcagag

SEQ ID NO: 104 - DNA, 60 bp, Artificial Sequence
  synthetic sequence
ccaccaccctcagagccgccaccagaaccaccaccagagccgccgccagcattgacagga

SEQ ID NO: 105 - DNA, 60 bp, Artificial Sequence
  synthetic sequence
ggttgaggcaggtcagacgattggccttgatattcacaaacaaataaatcctcattaaag

SEQ ID NO: 106 - DNA, 60 bp, Artificial Sequence
  synthetic sequence
ccagaatggaaagcgcagtctctgaatttaccgttccagtaagcgtcatacatggctttt

SEQ ID NO: 107 - DNA, 60 bp, Artificial Sequence
  synthetic sequence
gatgatacaggagtgtactggtaataagttttaacggggtcagtgccttgagtaacagtg

SEQ ID NO: 108 - DNA, 60 bp, Artificial Sequence
  synthetic sequence
cccgtataaacagttaatgccccctgcctatttcggaacctattattctgaaacatgaaa

SEQ ID NO: 109 - DNA, 60 bp, Artificial Sequence
  synthetic sequence
ccaggcggataagtgccgtcgagagggttgatataagtatagcccggaataggtgtatca

SEQ ID NO: 110 - DNA, 60 bp, Artificial Sequence
  synthetic sequence
ccgtactcaggaggtttagtaccgccaccctcagaaccgccaccctcagaaccgccaccc

SEQ ID NO: 111 - DNA, 60 bp, Artificial Sequence
  synthetic sequence
tcagagccaccaccctcattttcagggatagcaagcccaataggaacccatgtaccgtaa

SEQ ID NO: 112 - DNA, 60 bp, Artificial Sequence
  synthetic sequence
cactgagtttcgtcaccagtacaaactacaacgcctgtagcattccacagacagccctca

SEQ ID NO: 113 - DNA, 60 bp, Artificial Sequence
  synthetic sequence
tagttagcgtaacgatctaaagttttgtcgtctttccagacgttagtaaatgaattttct

SEQ ID NO: 114 - DNA, 60 bp, Artificial Sequence
  synthetic sequence
gtatgggattttgctaaacaactttcaacagtttcagcggagtgagaatagaaaggaaca

SEQ ID NO: 115 - DNA, 60 bp, Artificial Sequence
  synthetic sequence
actaaaggaattgcgaataataattttttcacgttgaaaatctccaaaaaaaaggctcca

SEQ ID NO: 116 - DNA, 60 bp, Artificial Sequence
  Synthetic sequence
aaaggagcctttaattgtatcggtttatcagcttgctttcgaggtgaatttcttaaacag

SEQ ID NO: 117 - DNA, 60 bp, Artificial sequence
  synthetic sequence
cttgataccgatagttgcgccgacaatgacaacaaccatcgcccacgcataaccgatata

SEQ ID NO: 118 - DNA, 60 bp, Artificial Sequence
  Synthetic sequence
ttcggtcgctgaggcttgcagggagttaaaggccgcttttgcgggatcgtcaccctcagc

SEQ ID NO: 119 - DNA, 60 bp, Artificial Sequence
  synthetic sequence
ctttttcatgaggaagtttccattaaacgggtaaaatacgtaatgccactacgaaggcac

SEQ ID NO: 120 - DNA, 60 bp, Artificial Sequence
  synthetic sequence
caacctaaaacgaaagaggcaaaagaatacactaaaacactcatctttgacccccagcga

SEQ ID NO: 121 - DNA, 60 bp, Artificial Sequence
  synthetic sequence
ttataccaagcgcgaaacaaagtacaacggagatttgtatcatcgcctgataaattgtgt

SEQ ID NO: 122 - DNA, 60 bp, Artificial Sequence
  synthetic sequence
cgaaatccgcgacctgctccatgttacttagccggaacgaggcgcagacggtcaatcata

SEQ ID NO: 123 - DNA, 60 bp, Artificial Sequence
  synthetic sequence
agggaaccgaactgaccaactttgaaagaggacagatgaacggtgtacagaccaggcgca

SEQ ID NO: 124 - DNA, 60 bp, Artificial sequence
  synthetic sequence
taggctggctgaccttcatcaagagtaatcttgacaagaaccggatattcattacccaaa

SEQ ID NO: 125 - DNA, 60 bp, Artificial Sequence
  synthetic sequence
tcaacgtaacaaagctgctcattcagtgaataaggcttgccctgacgagaaacaccagaa

SEQ ID NO: 126 - DNA, 60 bp, Artificial Sequence
  synthetic sequence
cgagtagtaaattgggcttgagatggtttaatttcaactttaatcattgtgaattacctt

SEQ ID NO: 127 - DNA, 60 bp, Artificial Sequence
  synthetic sequence
atgcgattttaagaactggctcattataccagtcaggacgttgggaagaaaaatctacgt

SEQ ID NO: 128 - DNA, 60 bp, Artificial Sequence
  Synthetic sequence
taataaaacgaactaacggaacaacattattacaggtagaaagattcatcagttgagatt

SEQ ID NO: 129 - DNA, 60 bp, Artificial sequence
  synthetic sequence
taagagcaacactatcataaccctcgtttaccagacgacgataaaaaccaaaatagcgag

SEQ ID NO: 130 - DNA, 60 bp, Artificial sequence
  synthetic sequence
aggcttttgcaaaagaagttttgccagagggggtaatagtaaaatgtttagactggatag

SEQ ID NO: 131 - DNA, 60 bp, Artificial sequence
  synthetic sequence
cgtccaatactgcggaatcgtcataaatattcattgaatccccctcaaatgctttaaaca

SEQ ID NO: 132 - DNA, 60 bp, Artificial Sequence
  synthetic sequence
gttcagaaaacgagaatgaccataaatcaaaaatcaggtctttaccctgactattatagt

SEQ ID NO: 133 - DNA, 60 bp, Artificial Sequence
  synthetic sequence
cagaagcaaagcggattgcatcaaaaagattaagaggaagcccgaaagacttcaaatatc

SEQ ID NO: 134 - DNA, 60 bp, Artificial sequence
  synthetic sequence
gcgttttaattcgagcttcaaagcgaaccagaccggaagcaaactccaacaggtcaggat

SEQ ID NO: 135 - DNA, 60 bp, Artificial sequence
  synthetic sequence
tagagagtacctttaattgctccttttgataagaggtcatttttgcggatggcttagagc

SEQ ID NO: 136 - DNA, 60 bp, Artificial sequence
  synthetic sequence
ttaattgctgaatataatgctgtagctcaacatgttttaaatatgcaactaaagtacggt

SEQ ID NO: 137 - DNA, 60 bp, Artificial sequence
  synthetic sequence
gtctggaagtttcattccatataacagttgattcccaattctgcgaacgagtagatttag

SEQ ID NO: 138 - DNA, 49 bp, Artificial sequence
  synthetic sequence
tttgaccattagatacatttcgcaaatggtcaataacctgtttagctat

SEQ ID NO: 139 - DNA, 60 bp, Artificial sequence
  synthetic sequence
aacatccaataaatcatacaggcaaggcaaagaattagcaaaattaagcaataaagcctc

SEQ ID NO: 140 - DNA, 60 bp, Artificial sequence
  synthetic sequence
gtgagcgagtaacaacccgtcggattctccgtgggaacaaacggcggattgaccgtaatg

SEQ ID NO: 141 - DNA, 60 bp, Artificial Sequence
  synthetic sequence
ttcttttcaccagtgagacgggcaacagctgattgcccttcaccgcctggccctgagaga

SEQ ID NO: 142 - DNA, 60 bp, Artificial sequence
  synthetic sequence
tctgtccatcacgcaaattaaccgttgtagcaatacttctttgattagtaataacatcac

SEQ ID NO: 143 - DNA, 60 bp, Artificial sequence
  synthetic sequence
attcgacaactcgtattaaatcctttgcccgaacgttattaattttaaaagtttgagtaa

SEQ ID NO: 144 - DNA, 60 bp, artificial sequence
  synthetic sequence
tgggttatataactatatgtaaatgctgatgcaaatccaatcgcaagacaaagaacgcga

SEQ ID NO: 145 - DNA, 60 bp, Artificial sequence
  synthetic sequence
gttttagcgaacctcccgacttgcgggaggttttgaagccttaaatcaagattagttgct

SEQ ID NO: 146 - DNA, 60 bp, Artificial sequence
  synthetic sequence
tcaaccgattgagggagggaaggtaaatattgacggaaattattcattaaaggtgaatta

SEQ ID NO: 147 - DNA, 60 bp, Artificial sequence
  synthetic sequence
gtattaagaggctgagactcctcaagagaaggattaggattagcggggttttgctcagta

SEQ ID NO: 148 - DNA, 60 bp, Artificial sequence
  synthetic sequence
agcgaaagacagcatcggaacgagggtagcaacggctacagaggctttgaggactaaaga

SEQ ID NO: 149 - DNA, 60 bp, Artificial sequence
  synthetic sequence
taggaataccacattcaactaatgcagatacataacgccaaaaggaattacgaggcatag

SEQ ID NO: 150 - DNA, 60 bp, Artificial sequence
  synthetic sequence
attttcatttggggcgcgagctgaaaaggtggcatcaattctactaatagtagtagcatt

SEQ ID NO: 151 - DNA, 20 bp, Artificial sequence
  synthetic sequence
tctgtccatcacgcaaatta

SEQ ID NO: 152 - DNA, 20 bp, Artificial sequence
  synthetic sequence
aattttaaaagtttgagtaa

SEQ ID NO: 153 - DNA, 20 bp, Artificial sequence
  synthetic sequence
tcgcaagacaaagaacgcga

SEQ ID NO: 154 - DNA, 20 bp, Artificial sequence
  synthetic sequence
tcgcaagacaaagaacgcga

SEQ ID NO: 155 - DNA, 20 bp, Artificial sequence
  synthetic sequence
tattcattaaaggtgaatta

SEQ ID NO: 156 - DNA, 20 bp, Artificial sequence
  synthetic sequence
tagcggggttttgctcagta

SEQ ID NO: 157 - DNA, 14 bp, Artificial sequence
  synthetic sequence
tctcatggcccttc

SEQ ID NO: 158 - DNA, 40 bp, Artificial sequence
  synthetic sequence
ctactaatagtagtagcattaacatccaataaatcataca

SEQ ID NO: 159 - DNA, 25 bp, Artificial Sequence
  synthetic sequence
tgtttgctttcgtgaaaactcgcat

SEQ ID NO: 160 - DNA, 24 bp, Artificial sequence
  synthetic sequence
acattaggtgcaataccgaaggca

(19) United States
(12) Patent Application Publication
(10) Pub. No.: US 2022/0073903 A1
(43) Pub. Date: Mar. 10, 2022

(54) METHODS OF PURIFYING RIBONUCLEIC ACID SPECIES

(71) Applicant: The Research Foundation for the State University of New York, Albany, NY (US)

(72) Inventors: Kenneth Halvorsen, Glenmont, NY (US); Lifeng Zhou, Albany, NY (US)

(21) Appl. No.: 17/368,718

(22) Filed: Jul. 6, 2021

Related U.S. Application Data
(60) Provisional application No. 63/048,563, filed on Jul. 6, 2020.

(51) Int. Cl. C12N 15/10

(52) U.S. Cl. CPC C12N 15/1024

(57) ABSTRACT

The present disclosure is directed to ribonucleic acid (RNA) isolation and purification. For example, the present disclosure relates to a method of purifying a single ribonucleic acid (RNA) species, including: isolating a DNA nanoswitch-target complex within a gel medium, wherein the DNA nanoswitch-target complex includes a DNA nanoswitch and a target-of-interest; digesting the DNA nanoswitch and the gel medium to form digested byproducts, and a free target-of-interest; and isolating the free target-of-interest, wherein the free target-of-interest is a single RNA species.

### CROSS-REFERENCE TO RELATED APPLICATIONS

The present disclosure claims priority or the benefit under 35 U.S.C. § 119 of U.S. provisional application Nos. 63/048,563 filed Jul. 6, 2020, entirely incorporated by reference.

### STATEMENT OF GOVERNMENTAL SUPPORT

This invention was made with governmental support under grant No. GM124720 awarded by the National Institution of Health. The government has certain rights in this invention.

### INCORPORATION OF THE SEQUENCE LISTING

The present application contains a sequence listing the content of which is hereby incorporated by reference.

### FIELD OF THE INVENTION

The present disclosure relates to the area of molecular biology and to nucleic acid compositions and methods of use thereof. More specifically, the present disclosure relates to methods of purifying nucleotides, or oligonucleotides such as ribonucleic acid (RNA) and species thereof.

### BACKGROUND

Purification is a cornerstone of RNA research, arguably beginning in 1868 when Friedrich Miescher achieved the first nucleic acid (“nuclein”) purification. Subsequently, many types of RNA with diverse functions have been discovered including messenger RNA (mRNA), catalytic ribozymes, self-splicing RNAs, and gene regulating RNAs. The importance of RNA in human health is now well appreciated, especially with many viruses having RNA as their genetic information carrier. Additionally, recent discoveries of microRNAs, long noncoding RNAs (lncRNAs) and chemically modified RNAs have reshaped the understanding of the importance of RNA in biological processes and diseases. The recent explosion of RNA research has made RNA purification increasingly important.

RNA purification is typically used to isolate all or most RNAs from a biological sample and is commonly done with organic extraction or spin columns. However, purification of specific RNAs is substantially more difficult, and magnetic beads-based purification is the only major approach for performing such isolations. The inventors have observed that single-stranded DNA (ssDNA) capture probes on beads are problematic in targeting specific RNA sequences in cell lysates or total RNA samples. For example, the bead substrate approach is complex, expensive, and has low yield and low specificity due to non-specific capture of RNA on the bead surface.

Prior art of interest includes U.S. Patent Publication No. 2018/0223344 (herein incorporated by reference) where it is suggested to use a DNA nanoswitch in purification, however the methodology does not contemplate releasing a bound target-of-interest from a DNA nanoswitch within a medium such as a gel medium. or simultaneously purifying one or more targets-of-interest using two or more different DNA nanoswitches while simultaneously releasing the one or more bound targets-of-interest from the nanoswitches and digesting the separation medium.

What is needed are methods of purifying targeted RNA molecules alone, or in combination from a mixture of cell components and additional nucleic acids. There is a continuing need for methods to detect and/or purify single species RNA.

### SUMMARY

The present disclosure relates to a method of purifying one or more single ribonucleic acid (RNA) species, including: isolating a DNA nanoswitch-target complex within a gel medium, wherein the DNA nanoswitch-target complex includes a DNA nanoswitch and a target-of-interest; digesting the DNA nanoswitch and the gel medium to form digested byproducts, and a free target-of-interest; and isolating the free target-of-interest, wherein the free target-of-interest is a single RNA species.

In some embodiments the present disclosure relates to a method of purifying two or more single ribonucleic acid (RNA) species, including: isolating at least a first DNA nanoswitch-target complex and a second DNA nanoswitch-target complex within a gel medium, wherein the first DNA nanoswitch-target complex includes a first DNA nanoswitch and a first target-of-interest and the second DNA nanoswitch-target complex includes a second DNA nanoswitch and a second target-of-interest; digesting the first DNA nanoswitch, second DNA nanoswitch, and gel medium to form digested byproducts, a first free target-of-interest, and a second free target-of-interest; and isolating the first free target-of-interest and the second free target-of-interest, wherein the first free target-of-interest and the second free target-of-interest are different single RNA species.

In some embodiments, the present disclosure relates to a method of purifying a single ribonucleic acid (RNA) species, including: contacting a deoxyribonucleic acid (DNA) nanoswitch and an RNA target to form a DNA nanoswitch-RNA target complex; isolating the DNA nanoswitch-RNA target complex within a medium; freeing the RNA target from the DNA nanoswitch-RNA target complex to form free RNA; and isolating the free RNA, wherein the free RNA is a single RNA species. In some embodiments, the free RNA is a messenger RNA (mRNA), a catalytic ribozyme, a self-splicing RNA, or a gene regulating RNA.

The illustrative aspects of the present disclosure are designed to solve the problems herein described and/or other problems not discussed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 is a flowchart of a method of purifying a single ribonucleic acid (RNA) species in accordance with the present disclosure.

FIG. 2A shows the design and operation of the two-state DNA nanoswitch. A double stranded DNA is made with a single-stranded scaffold, complementary backbone oligos, and detector strands that can be addressably inserted at different locations. Addition of the key oligonucleotide binds the overhangs of the two detector regions ‘a’ and ‘b’ thereby forming a loop. This conformational change can be read out using gel electrophoresis.

FIG. 2B shows the sequence specificity of DNA nanoswitches. An agarose gel showing the sequence specificity of the nanoswitch. Switch A turns on only in the presence of key oligonucleotide A and switch B turns on only in the presence of key oligonucleotide B with no background detection of the incorrect strand.

FIG. 2C shows the loop size configuration of the on state. The left panel shows detector positions on the nanoswitch are shown in green. The middle panel shows the combination of two positions, which gives different loop sizes on recognition of the key oligonucleotide. The right panel shows that different loop sizes can be identified using a gel read out. Larger loop sizes provide a shorter read-out time.

FIG. 2D shows data relating to the limit of detection using nanoswitches.

FIGS. 3A, 3B, 3C, 3D, 3E, and 3F are schematic illustrations depicting: FIG. 3A the challenge of single species RNA purification; FIG. 3B a DNA nanoswitch converting from a linear to looped form in the presence of target RNA; FIG. 3C the purification workflow including detecting (steps 1-2) and purifying (steps 3-5) RNA; FIG. 3D a proof-of-concept validation for the purification of an mRNA fragment in total RNA with nanoswitch re-detection and verification by qRT-PCR; FIG. 3E shows the quantified purification yields of mRNA fragment and miR-206 based on qRT-PCR tests; and FIG. 3F depicts multiplexed purification of mRNA fragment and miR-206 in single reaction, wherein from left to right, the process flow shows the multiplexed detection, purification and verification by redetection without cross contamination.

FIGS. 4A, 4B, 4C, 4D, and 4E are schematic illustrations depicting: FIG. 4A components of eukaryotic ribosome; FIG. 4B multiplexed detection showing separate detection of 5.8S and 5S rRNAs as different loop sizes; FIG. 4C multiplexed purification demonstrating individual isolation of the 5.8S and 5S rRNA molecules without observable cross-contamination; FIG. 4D shows a process flow of purification and verification by LC-MS/MS of modified RNAs; FIG. 4E shows modification probing of the purified RNA molecules by LC-MS/MS, wherein Left: m^{4,4}C modification standard and right: purified RNA from 10 nM sample, and wherein the target RNA molecule has a m^{4,4}C modification on a cytosine (sequence shown above graph on right, * indicates position of modification).

FIGS. 5A, and 5B depict a schematic illustration and data relating to digestion of DNA nanoswitches in gel pieces. FIG. 5A is a schematic illustration of digesting DNA nanoswitches in agarose gel pieces by DNase I. FIG. 5B is data referring to digestion of DNA nanoswitches in 0.8% (left) and 3.2% (right) agarose gel pieces, wherein 10 ng DNA nanoswitch was pre-stained by 1×GelRed (Biotium, Inc.) and was loaded to each well. The gel bands of nanoswitches were cut out carefully to ensure similar size and then gel images were taken for reference (shown as inset in the figure). Each gel piece was submerged in 100 μl 1×DNase I reaction buffer (NEB, Inc.) in 1.5 ml tube and then 2U DNase I (NEB, Inc.) was added. Then, all samples were incubated at 37° C. Gel images were taken after incubation for 15, 30, 45 and 60 min.

FIGS. 6A, 6B, 6C, 6D, provide data relating to optimization of the capture probe and sensitivity test of DNA nanoswitch of mRNA fragment. FIG. 6A shows the detection test of the nanoswitch with different capture probe lengths by using corresponding DNA targets shows that the 20 nt probe has the highest detection efficiency. FIG. 6B shows detection sensitivity test of the nanoswitch with 20 nt capture probes by using corresponding DNA targets shows a detection limit as low as 3.1 pM. FIG. 6C shows testing different lengths of capture probes to detect an mRNA fragment. FIG. 6D shows sensitivity of detecting mRNA fragment using a nanoswitch with 20 nt capture probes.

FIGS. 7A and 7B depict a schematic illustration and data relating to proof-of-concept single species RNA purification. FIG. 7A shows detection, purification and redetection of mRNA fragment and miR-206 in water, finished in triplicate (lanes 1, 2 and 3). FIG. 7B shows detection, purification and redetection of mRNAfragment and miR-206 in total RNA, finished in triplicate (lanes 1, 2 and 3). The frames indicate the areas of gel images shown in FIG. 3D. Each pre-purification sample (for each lane) has 30 μl volume that contains 0.53 nM nanoswitch (mRNA fragment) or 0.6 nM nanoswitch (miR-206), 3.3 nM target RNA, 10 mM MgCl₂, and 1×PBS. 250 ng HeLa total RNA was added for the test within total RNA. Redetection samples contain 10 μL volume with 0.15 nM nanoswitch, 4 μL purified RNA sample, 10 mM MgCl₂, 1×PBS, and 200 nM blocking oligos.

FIG. 8 shows data relating to length validation for purified mRNA fragment. FIG. 8 is a gel image showing the amplicons of qRT-PCR of the purified mRNA fragment is shown in lane L3, alongside the dsDNA template in lane L2 and a 100 bp ladder in lane L1. The gel was 1.6% agarose gel, run in cold room at 60V for 60 min. The frame indicates the areas of gel images shown in FIG. 3D.

FIGS. 9A and 9B show data relating to quantification of the purification yield of mRNA based on qRT-PCR test. FIG. 9A shows one of three gel images of mRNA fragment detection. For each test, the detection bands of four lanes (1, 2, 3 and 4) were combined, each containing 10 fmol nanoswitches and 10 fmol target mRNA fragment with 10 mM MgCl₂ and 1×PBS in a 10 μL volume. The results of qRT-PCR of the three independently purified products is shown in the table. Each purified sample was quantified three times by qRT-PCR (test 1 to 3) and the average concentration is the average value of the three tests. FIG. 9B shows equations used to calculate the recovery yield and overall yield. The data shown in FIG. 3E is summarized in the table on the bottom.

FIGS. 10A and 10B depict data relating to quantification of the purification yield of miR-206 based on qRT-PCR test. FIG. 10(A) depicts of one of three gel images of miR-206 detection. For each test, the detection bands of four lanes (1, 2, 3 and 4) were combined, each containing 10 fmol nanoswitches and 10 fmol target microRNA with 10 mM MgCl₂ and 1×PBS in a 10 μL volume. The results of qRT-PCR of the three independently purified products is shown in the table. Each purified sample was quantified three times (test 1 to 3) and average concentration is the average value of the three tests. FIG. 10B depicts equations used to calculate the recovery yield and overall yield. μl*nM: volume×concentration. The data shown in FIG. 3E is summarized in the table on the bottom.

FIGS. 11A-11E depict gel images of the multiplexed purification of mRNA fragment and miR-206. FIG. 11A depicts loop sizes of DNA nanoswitches for mRNA fragment and miR-206 detection. FIG. 11B shows the entire gel image of the multiplexed detection of mRNA fragment and miR-206 in water. The gel was a 0.8% agarose gel and was run in the cold room at 60 V for 2 hours. FIG. 11C shows the entire gel image of the multiplexed detection of mRNA fragment and miR-206 for purification. The gel was a 0.8% agarose gel and was run in the cold room at 65 V for 2 hours. Totally, 60 μl detection sample (containing 0.8 nM unpurified nanoswitch for each target RNA, 10 nM of each target RNA, 10 mM MgCl₂, 1×PBS) was prepared and 10 μl was loaded to each well. (FIGS. 11D-E) Redetection of the mRNA fragment and miR-206 purified from the detection bands shown in FIG. 11C. The dotted frames indicate the areas of gel images shown in FIG. 3F.

FIGS. 12A and 12B depict data relating to the detection and purification of 5.8S and 5S rRNA. FIG. 12A shows negative control and detection test of nanoswitches designed for 5.8S and 5S rRNA. Detection was validated using DNA controls. FIG. 12B shows he entire gel image of the 5.8S and 5S rRNA multiplexed detection. Totally, 80 μl sample (containing 0.75 nM nanoswitch of each target RNA, 50 ng/μl total RNA of HeLa cell, 10 mM MgCl₂, 1×PBS, 3.3×GelRed) was prepared and incubated with a thermal annealing ramp (40° C. to 25° C. over 12 hours) and 10 μl was loaded to each well. For each target, two clean columns were used and 40 μl purified sample was obtained.

FIGS. 13A-13D depict data relating to purification of RNA with chemical modification. FIG. 13A and FIG. 13B show optimizing design of DNA nanoswitches for the detection of RNA with chemical modification m^{4,4}C. The nanoswitch with 15 nt capture probe length has higher detection efficiency compared to the nanoswitch with 10 nt capture probes. FIG. 13C shows PEG purification of DNA nanoswitch and the negative control and detection test. FIG. 13D shows gel images showing detection of chemically modified RNA (at 1 and 10 nM concentration) when spiked into total RNA. For each case, 80 μl detection sample (containing 4 nM nanoswitch, 50 ng/μl total RNA of HeLa cell, 10 mM MgCl₂, 1×PBS, 3.3×GelRed) was prepared and incubated with a thermal annealing ramp (40° C. to 25° C. over 12 hours) and 10 μl was loaded to each well. * indicates the possible complexes formed by two or more nanoswitches, which is known to occur at high nanoswitch concentration as in this case.

FIG. 14 depicts data relating to purification of RNA molecules with chemical modifications such as modification probing of the purified RNA molecules by LC-MS/MS, top: m^{4,4}C modification standard, middle and bottom are the interrogations of RNA purified from 10 nM and 1 nM samples respectively.

FIGS. 15A-15C depict data relating to test of DNA gel extraction spin columns. FIG. 15A shows after cutting the gel bands, Freeze 'N Squeeze DNA gel spin columns (Bio-Rad Laboratories, Inc.) were used to extract RNA-looped DNA nanoswitches for the next purification steps. FIG. 15B shows two tests were conducted and 20 μl purified RNA sample was obtained for each. For verification by redetection, 5 μl purified RNA sample was used. Comparison of the gel band intensities of redetection and initial detection bands shows that the purification yield is low for both tests. FIG. 15C shows, in step 2, concentrating the sample extracted from the excised gel pieces in a universal vacuum system (Savant UVS 400) and noticed that after concentration, white powder appeared on the inner wall of the tube. The white powder is believed to be agarose which could influence the following purification steps and result in low purification yield.

FIG. 16 is a flowchart of a method of purifying a single ribonucleic acid (RNA) species in accordance with the present disclosure.

FIG. 17 is a flowchart of a method of purifying a single ribonucleic acid (RNA) species in accordance with the present disclosure.

### DETAILED DESCRIPTION

The compositions and methods of the present disclosure herein relate to detecting, isolating, and/or purifying one or more targets-of-interest, such as one or more ribonucleic acid molecules. In embodiments, the present disclosure relates to a method of purifying a single ribonucleic acid (RNA) species, including: isolating a DNA nanoswitch-target complex within a gel medium, wherein the DNA nanoswitch-target complex includes a DNA nanoswitch and a target-of-interest; digesting the DNA nanoswitch and the gel medium to form digested byproducts, and a free target-of-interest; and isolating the free target-of-interest, wherein the free target-of-interest is a single RNA species.

Embodiments of present disclosure advantageously provide improved methods, compositions, and assays for the detection, identification, or purification of one or more targets-of-interest such as one or more ribonucleic acid (RNA) molecules e.g., single species RNA, or species thereof. Additional benefits of the methods and compositions of the present disclosure may include purifying two or more targets-of-interest from a mixture including biological components such as a plurality of digested byproducts, nucleic acids, DNAs, RNA, proteins, and fragments thereof. Advantages may be especially apparent where it is desirable to purify multiple targets-of-interest using two or more different nanoswitches, or where process efficiencies are increased by releasing a bound target-of-interest from a DNA nanoswitch within a medium such as a gel medium, or where it is desirable to purifying targeted RNA molecules alone, or in combination from a mixture of cell components and additional nucleic acids.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein, the terms “bind” and “binding” generally refer to the non-covalent interaction between a pair of partner molecules or portions thereof that exhibit mutual affinity or binding capacity. In embodiments, binding can occur such that the partners are able to interact with each other to a substantially higher degree than with other, similar substances. This specificity can result in stable complexes that remain bound during handling steps such as chromatography, centrifugation, filtration, and other techniques typically used for separations and other processes.

The terms “deoxyribonucleotide” and “DNA” refer to a nucleotide or polynucleotide including at least one ribosyl moiety that has an H at the 2′ position of a ribosyl moiety. In embodiments, a deoxyribonucleotide is a nucleotide having an H at its 2′ position

By “hybridizable” or “complementary” or “substantially complementary” a nucleic acid (e.g. RNA, DNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (e.g., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA nanoswitch base pairs with a target RNA, etc.): G can also base pair with U. For example, G/U base-pairing is partially responsible for the degeneracy (i.e., redundancy) of the genetic code in the context of tRNA anti-codon base-pairing with codons in mRNA. In embodiments, hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “elute” and “eluting” refer to the disruption of non-covalent interactions between partner molecules such that the partners become unbound from one another. In embodiments, the disruption can be effected via introduction of a competitive binding species, or via a change in environmental conditions (e.g., ionic strength, pH, or other conditions).

As used herein, the term “forming a mixture” refers to the process of bringing into contact at least two distinct species such that they mix together and interact. “Forming a reaction mixture” and “contacting” refer to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture. “Conversion” and “converting” refer to a process including one or more steps wherein a species is transformed into a distinct product.

An “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The term “nucleotide” refers to a ribonucleotide or a deoxyribonucleotide or modified form thereof, as well as an analog thereof.

The term “nanoswitch” refers to a nucleic acid complex for use in detecting or binding to a target. A nanoswitch is typically a nucleic acid molecule, either single- or double-stranded, which is modified to contain segments of nucleic acids in a manner that would not otherwise exist in nature, and designed to assume a linear (or open) conformation in the absence of target and assume a looped (or closed) formation in the presence of a target.

Nucleic acid construct: The term “nucleic acid construct” as used herein refers to a nucleic acid molecule, either single- or double-stranded, which is isolated from a naturally occurring gene, or which is synthesized or modified to contain segments of nucleic acids in a manner that would not otherwise exist in nature.

As used herein, the term “nucleic acid molecule” refers to any molecule containing multiple nucleotides (e.g., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As described further below, bases include C, T, U, C, and G, as well as variants thereof. As used herein, the term refers to ribonucleotides (including oligoribonucleotides (ORN)) as well as deoxyribonucleotides (including oligodeoxynucleotides (ODN)). The term shall also include polynucleosides (i.e., a polynucleotide minus the phosphate) and any other organic base containing polymer. Nucleic acid molecules can be obtained from existing nucleic acid sources (e.g., genomic or cDNA), but include synthetic (e.g., produced by oligonucleotide synthesis). In embodiments, the terms “nucleic acid” “nucleic acid molecule” and “polynucleotide” may be used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

In embodiments, the term “oligonucleotide” refers to a polynucleotide of between 4 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized.

Expression vector: The term “expression vector” is defined herein as a linear or circular DNA molecule that comprises a polynucleotide encoding a polypeptide, and which is operably linked to additional nucleotides that provide for its expression.

The term “isolated” means a substance in a form or environment that does not occur in nature. Non-limiting examples of isolated substances include (1) any non-naturally occurring substance, (2) any substance such as an nucleic acid, RNA, DNA, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated.

The terms “sequence identity”, “identity” and the like as used herein with respect to polynucleotide or polypeptide sequences refer to the nucleic acid residues or amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polynucleotide or polypeptide sequence in the comparison window may include additions or deletions (e.g., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity.

It would be understood that, when calculating sequence identity between a DNA sequence and an RNA sequence, T residues of the DNA sequence align with, and can be considered “identical” with, U residues of the RNA sequence. For purposes of determining “percent complementarity” of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs.

In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety. In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm, and the sequence identity is calculated by identifying the number of exact matches identified by the program divided by the “alignment length”, where the alignment length is the length of the entire alignment including gaps and overhanging parts of the sequences. In embodiments, the mafft alignment program is suitable for use herein.

The term “recombinant” when used herein to characterize a nucleic acid sequence such as a plasmid, vector, or construct refers to an artificial combination of two otherwise separated segments of sequence, e.g., by chemical synthesis and/or by manipulation of isolated segments of nucleic acids by genetic engineering techniques.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

“Substantially similar” refers to nucleic acid molecules wherein changes in one or more nucleotide bases result in substitution of one or more amino acids, but do not affect the functional properties of the protein encoded by the DNA sequence. “Substantially similar” also refers to nucleic acid molecules wherein changes in one or more nucleotide bases do not affect the ability of the nucleic acid molecule to mediate alteration of gene expression by antisense or co-suppression technology. “Substantially similar” also refers to modifications of the nucleic acid molecules of the instant disclosure (such as deletion or insertion of one or more nucleotide bases) that do not substantially affect the functional properties of the resulting transcript vis-a-vis the ability to mediate alteration of gene expression by antisense or co-suppression technology or alteration of the functional properties of the resulting protein molecule. The disclosure encompasses more than the specific exemplary sequences.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### Certain Embodiments of the Present Disclosure

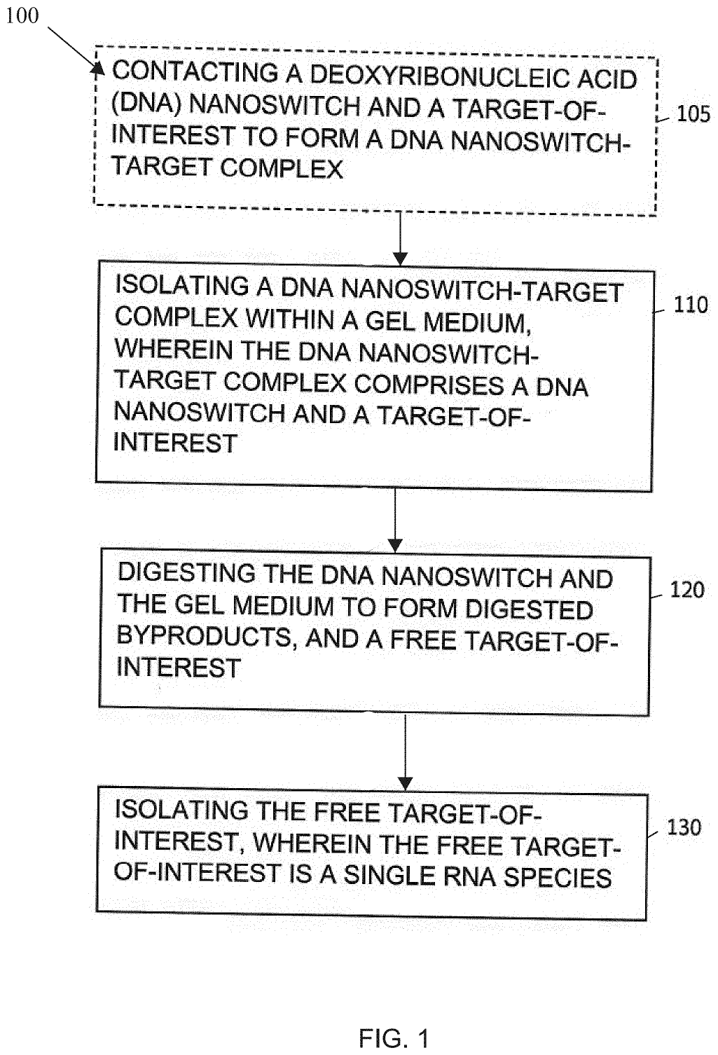

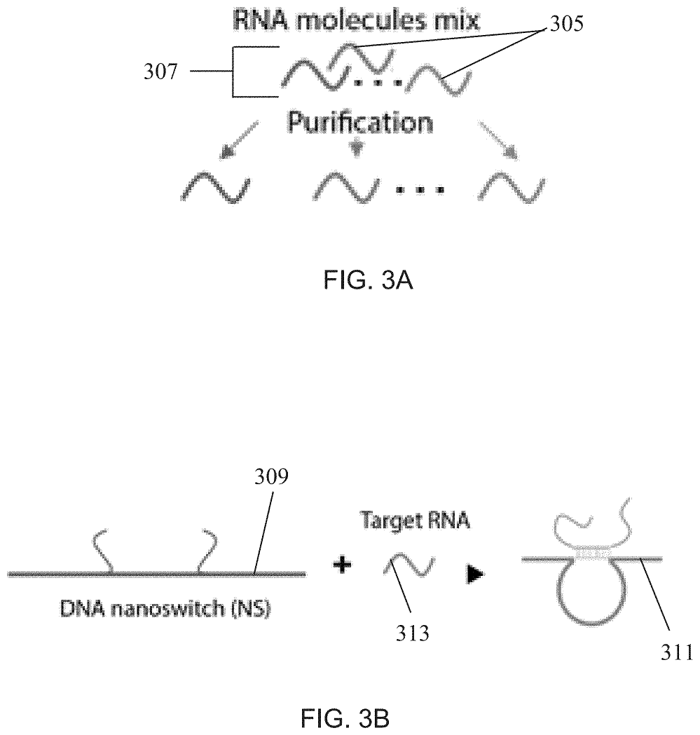

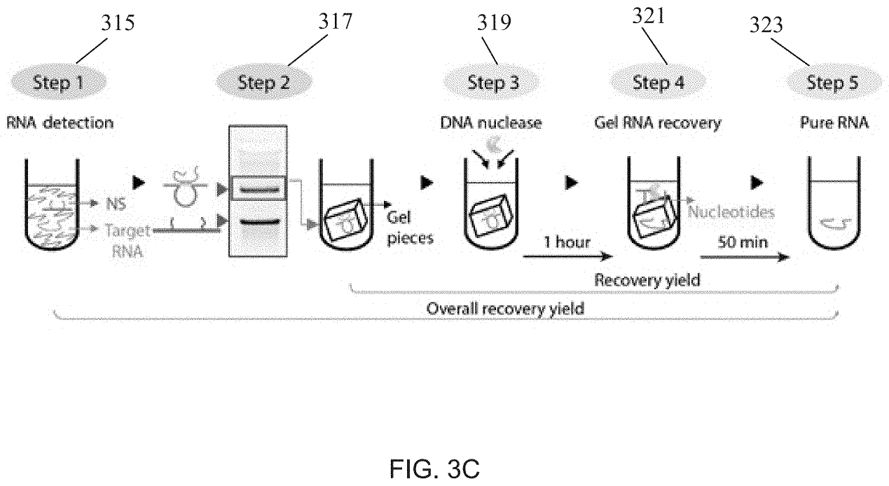

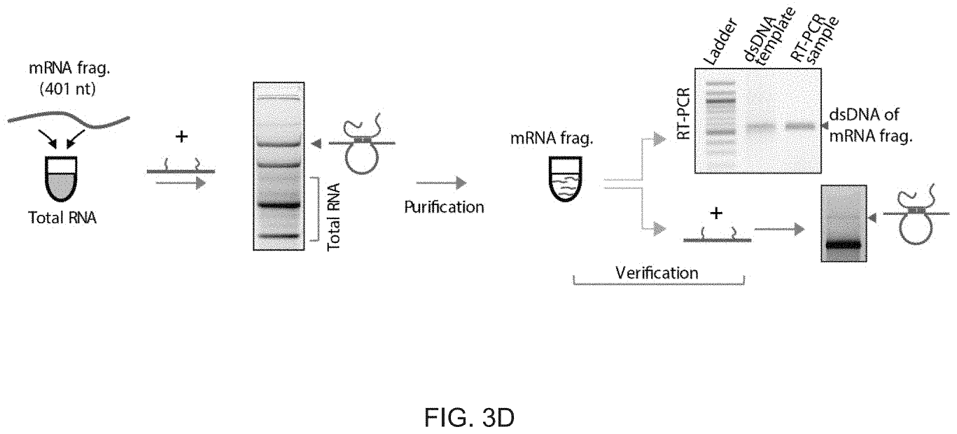

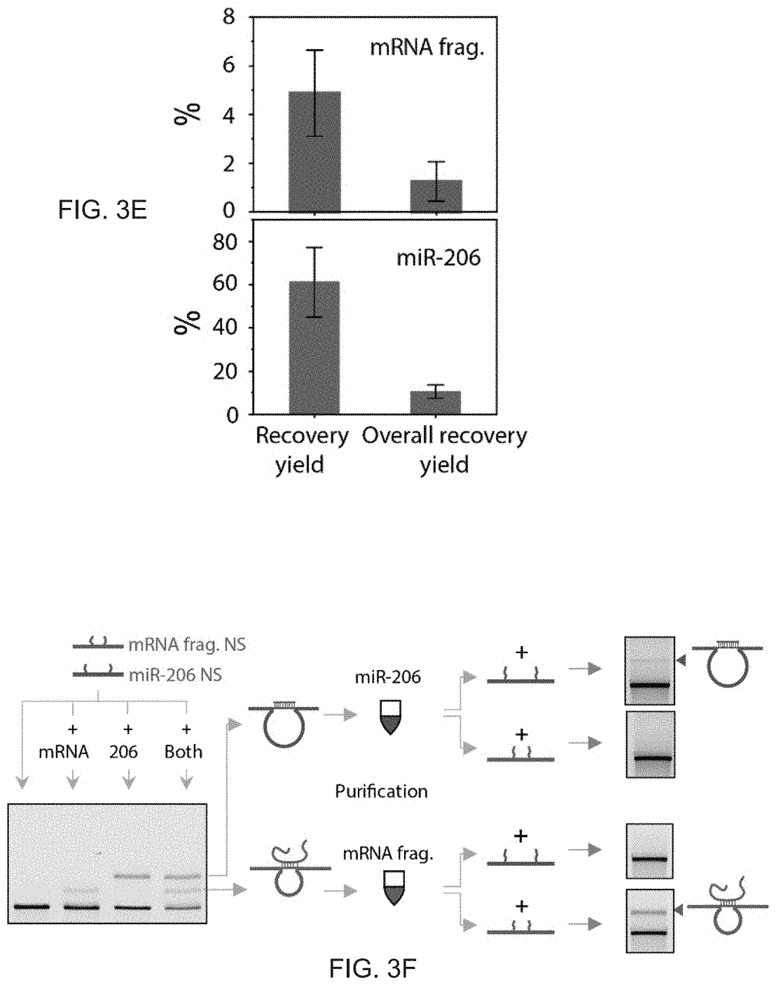

In embodiments, the present disclosure relates to one or more methods of isolating or providing one or more substantially purified target(s)-of-interest such as one or more ribonucleic acid (RNA) molecules. FIG. 1 is a flow diagram of a method 100 for a method of purifying a single ribonucleic acid (RNA) species in a sample in accordance with some embodiments of the present disclosure. The method 100 is described below with respect to the stages of processing as depicted in, e.g., FIGS. 3A-3C and may be performed, for example, in a suitable labware, such test tubes as shown below.

Initially, prior to process sequence 110, the method 100 may optionally include at process sequence 105 contacting a deoxyribonucleic acid (DNA) nanoswitch and a target-of-interest to form a DNA nanoswitch-target complex. For example, where it is desirable to target RNA, a preselected deoxyribonucleic acid (DNA) nanoswitch may be contacted with a desired RNA target to form a DNA nanoswitch-RNA target complex. In embodiments, one or more DNA nanoswitches are preformed or preselected to combine with a preselected target-of-interest such as a preselected RNA target. In some embodiments, DNA nanoswitches are preformed or preselected to includes hybridizable, or complementary, or substantially complementary nucleic acids, or nucleic acid molecules, including or consisting of one or more segments of nucleotides that enables it to non-covalently bind, e.g. form Watson-Crick base pairs and/or G/U base pairs, anneal, or hybridize, to one or more RNA targets, or preselected RNA targets in a sequence-specific, antiparallel, manner, or combine with a preselected target-of-interest such as a preselected RNA target. In embodiments, a deoxyribonucleic acid (DNA) nanoswitch includes one or more segments including predetermined nucleotides ordered to combine with, alone, or in combination, an RNA target-of-interest or a predetermined RNA.

In embodiments, DNA nanoswitch suitable for use herein is a nucleic acid complex for use in detecting targets. Targets are detected based on their binding interactions with the nanoswitches and the conformational changes that are induced in the nanoswitches as result of such binding. The nanoswitches are designed so that in the absence of the target they typically assume a linear (or open) conformation and in the presence of target they assume a looped (or closed) conformation. These conformations are detected and physically separable from each other using various techniques including but not limited to gel electrophoresis. In the context of gel electrophoresis, the open and closed conformations migrate to different extents through a gel, and they can be excised from the gel in order to, in some embodiments, further purify the target that is bound to the nanoswitch. It is envisioned that one can avoid damage to an RNA target during the purification process, by using DNA dyes that excite in blue light rather than UV responsive dyes. Suitable dyes include GelGreen, SYBR Green, SYBR Safe, and EvaGreen. See e.g., reference to Biorxiv reference: located on the world wide web at www.biorxiv.org/content/10.1101/2020.07.07.191338v1.abstract.

In embodiments, nanoswitches of the present disclosure are designed to detect a variety of targets, including but not limited to targets that are nucleic acids or proteins or peptides. Typically, the nanoswitch includes or is bound to a binding partner for a target of interest. In embodiments, the binding partner may be a preselected nucleic acid that binds to a target that is a nucleic acid (such as RNA) based on sequence complementarity. In embodiments, and as shown in FIG. 3C, nanoswitches of the present disclosure bind to a pre-selected portion of a target such as a target-of-interest, and portions of the target may not be bound to the nano-switch.

Various aspects of the present disclosure relate to the use of nanoswitches to detect targets-of-interest that are nucleic acids, such as RNA target nucleic acids. In some embodiments, the target comprises or consist of ribonucleic acid. In some embodiments, the ribonucleic acid is a messenger RNA (mRNA), a catalytic ribozyme, a self-splicing RNA, or a gene regulating RNA. In some embodiments, the targets-of-interest include one or more ribonucleic acids selected from the group including messenger RNA (mRNA), a catalytic ribozyme, a self-splicing RNA, a gene regulating RNA, microRNA, ribosomal RNA, viral RNA, long noncoding RNA, and chemically modified RNA. In embodiments, the target is characterized as an oligonucleotide, a polynucleotide of between 4 and 100 nucleotides of single- or double-stranded nucleic acid (e.g., DNA, RNA, or a modified nucleic acid). In embodiments, the target is characterized as an oligonucleotide. In embodiments, the target is characterized as an RNA oligonucleotide of 4 and 100 nucleotides. In embodiments, the target is characterized as a single-stranded RNA oligonucleotide of 4 and 100 nucleotides.

In embodiments, detection of nucleic acids such as RNA is important for a variety of applications including for example in the fields of medicine and forensics. In some embodiments, the present disclosure provides a programmable nucleic acid-based nanoswitch that undergoes a pre-defined conformational change upon binding a target nucleic acid such as target RNA, converting the nanoswitch from a linear “off” state (or conformation) to a looped “on” state (or conformation). In embodiments, the looped “on” state is a relates to a DNA complex or conformation that includes a combination of the DNA nanoswitch to the target forming a DNA nanoswitch-target complex of the present disclosure.

In embodiments, a DNA nanoswitch-target complex can be detected using separation techniques such as standard gel electrophoresis, which are capable of physically separating the open and closed conformations from each other and from other components in a mixture, and in some instances also are capable of facilitating isolation of the nanoswitch and its bound target, e.g. the DNA nanoswitch-target complex. In embodiments, other separation medium suitable for use herein may include liquid chromatography medium such as those used HPLC columns, or other medium such as those used in capillary electrophoresis.

In embodiments, the present disclosure demonstrates successful detection of a single target ribonucleic acid from a randomized pool of high concentration oligonucleotides with no false positive detection. The detection method can be accomplished quickly, including as demonstrated herein within 30 minutes from sample mixture to readout. The approach is a low cost and technically accessible, and thus well-suited for point-of-use detection.

In addition, the RNA complexes may also be used to simultaneously detect more than one target RNA. For example, the nucleic acid complex may be designed to hybridize to one, two or more target RNAs, with a different, discernable structure resulting from each such as wherein the ribonucleic acid is a messenger RNA (mRNA), a catalytic ribozyme, a self-splicing RNA, or a gene regulating RNA.

In embodiments, a nucleic acid complex such as a nanoswitch, as described herein includes a scaffold nucleic acid hybridized in a sequence specific manner to a plurality of oligonucleotides. The scaffold and the oligonucleotides may be referred to herein as being single-stranded. In embodiments, prior to hybridization to each other, both nucleic acid species are single-stranded. In embodiments, upon hybridization, a double-stranded nucleic acid is formed. Typically, the oligonucleotides hybridize to the scaffold nucleic acid in a consecutive, non-overlapping, manner.

In some non-limiting embodiments, the nucleic acid complexes are formed by hybridizing a scaffold nucleic acid to one or more oligonucleotides. The disclosure contemplates any variety of means and methods for generating the nucleic acid complexes described herein. It is also to be understood that while for the sake of brevity the disclosure refers to oligonucleotides that are hybridized to a scaffold nucleic acid, such a complex may have been formed by hybridizing single stranded scaffold to single stranded oligonucleotides, but it is not intended that it was exclusively formed in this manner. In embodiments, the nucleic acid complexes may include double-stranded and single-stranded regions. As used herein, a double-stranded region is a region in which all nucleotides on the scaffold are hybridized to their complementary nucleotides on the oligonucleotide. Double-stranded regions may include “single-stranded nicks” as the hybridized oligonucleotides typically are not ligated to each other. The single-stranded regions are scaffold sequences that are not hybridized to oligonucleotides. Certain complexes may include one or more single-stranded regions in between double-stranded regions (typically as a result of unhybridized nucleotides in between adjacent hybridized oligonucleotides). The complexes may be at least 50%, at least 60%, at least 70%, at least 80%, at least 90%, at least 95%, at least 96%, at least 97%, at least 98%, at least 99%, or 100% double-stranded. In some embodiments, they are at least 80% double stranded.

In embodiments, the DNA nanoswitch or nucleic acid complexes are modular complexes to which can be attached one or more targets of interest, one or more binding pairs of interest, and the like. The terms attach, link and conjugate are used interchangeably throughout this disclosure unless otherwise stated.

The nanoswitches provided herein are stable in complex fluids such as but not limited to serum-containing samples, including up to 30% FBS. In some embodiments, nanoswitches for use herein are configured to convert from unbound to bound forms in the presence of complex fluids (e.g., 30% FBS). Moreover, the nanoswitches are also stable for an extended period of time. Once synthesized, the nanoswitches may be dried and stored for days, weeks or months.

In some embodiments, the nucleic acid complexes can be made using nucleic acid nanostructure techniques such as but not limited to DNA origami. (Rothemund P. W. K. (2006) Nature 440: 297-302; Douglas S. M. et al. (2009) Nature 459: 414-8). In embodiments, the nucleic acid complexes may be formed as described in U.S. Patent Publication No. 2018/0223344 entitled Compositions and Methods for Analyte Detection Using Nanoswitches published on 9 Aug. 2018 to Chandrasekaren et al. (herein entirely incorporated by reference). In embodiments, the nucleic acid complexes can be formed as described in U.S. Pat. No. 9,914,958 entitled Nucleic Acid-Based Linkers For Detecting And Measuring Interactions (herein entirely incorporated by reference).

### Scaffolds

In embodiments, scaffold nucleic acid suitable for use herein may be of any length sufficient to allow association (e.g., binding) and dissociation (e.g., unbinding) of binding partners to occur and to be distinguished from other association and/or dissociation events using the read-out methods provided herein, including gel electrophoresis.

In embodiments, the scaffold nucleic acid is at least 500 nucleotides in length, and it may be as long as 50,000 nucleotides in length (or it may be longer). The scaffold nucleic acid may therefore be 1000-20,000 nucleotides in length, 1000-15,000 nucleotides in length, 1000-10,000 in length, or any range therebetween. In some embodiments, the scaffold ranges in length from about 5,000-10,000 nucleotides, and may be about 7000-7500 nucleotides in length or about 7250 nucleotides in length.

In some embodiments, the scaffold may be a naturally occurring nucleic acid (e.g., M13 scaffolds such as M13mp18). M13 scaffolds are disclosed by Rothemund 2006 Nature 440:297-302, the teachings of which are incorporated by reference herein. Such scaffolds are about 7249 nucleotides in length.

In some embodiments, the scaffold nucleic acid may also be non-naturally occurring nucleic acids such as polymerase chain reaction (PCR)-generated nucleic acids, rolling circle amplification (RCA)-generated nucleic acids, etc. In some embodiments, the scaffold nucleic acid is rendered single-stranded either during or post synthesis. Methods for generating a single-stranded scaffold include asymmetric PCR. Alternatively, double-stranded nucleic acids may be subjected to strand separation techniques in order to obtain the single-stranded scaffold nucleic acids. The scaffold nucleic acid may comprise DNA, RNA, DNA analogs, RNA analogs, or a combination thereof, provided it is able to hybridize in a sequence-specific and non-overlapping manner to the oligonucleotides. In some instances, the scaffold nucleic acid is a DNA.

### Oligonucleotides

In embodiments, the scaffold nucleic acid is hybridized to a plurality of oligonucleotides. Each of the plurality of oligonucleotides is able to hybridize to the scaffold nucleic acid in a sequence-specific and non-overlapping manner (i.e., each oligonucleotide hybridizes to a distinct sequence in the scaffold). The length and the number of oligonucleotides used may vary. In some instances, the length and sequence of the oligonucleotides is chosen so that each oligonucleotide is bound to the scaffold nucleic acid at a similar strength. This is important if a single condition is used to hybridize a plurality of oligonucleotides to the scaffold nucleic acid, such as for example in a one-pot synthesis scheme.

In embodiments, the number of oligonucleotides will depend in part on the application, the length of the scaffold, and the length of the oligonucleotides themselves. In embodiments, the oligonucleotides are designed to be of approximately equal length. In some embodiments, the oligonucleotides may be about 20-100 nucleotides in length. The oligonucleotides may be, without limitation, about 20, about 30, about 40, about 50, about 60, about 70, about 80, about 90 or about 100 nucleotides in length. In some embodiments, the oligonucleotides may be about 40-80 nucleotides in length. In some embodiments, the oligonucleotides may be about 60 nucleotides in length.

The number of oligonucleotides in the plurality may be about 70, about 80, about 90, about 100, about 110, about 120, about 130, about 140, about 150, about 160, about 170, about 180, about 190, about 200, about 300, about 400, about 400, about 500, about 600, about 700, about 800, about 900, or about 1000, without limitation.

In some embodiments and as described in the Examples, the nucleic acid complex may include the M13 ssDNA as the scaffold and about 120 oligonucleotides each equal to or about 60 nucleotides in length.

In embodiments, the oligonucleotides may be characterized as modified or unmodified or variable oligonucleotides. In embodiments, the variable oligonucleotides may be conjugated to reactive groups that are not normally present in a nucleic acid sequence, such as for example click chemistry reactive groups, or they may be conjugated to target-specific binding partners such as antibodies or antibody fragments, or they may comprise other moieties which are not typically present in an unmodified oligonucleotide. An example is a variable oligonucleotide comprising a phosphate at their 5′ end (referred to herein as a 5′ phosphate). Oligonucleotides having this latter modification are used herein in the detection of target nucleic acids, and in this context such oligonucleotides are referred to as “detector” strands since they detect the target nucleic acid via hybridization.

In some instances, the first and last oligonucleotides as well as “internal” oligonucleotides, typically at pre-defined positions along the length of the scaffold, may be modified oligonucleotides. The position of the variable oligonucleotides may be, but are not necessarily, evenly distributed along the length of the scaffold.

### Binding Interactions and Looped Conformations

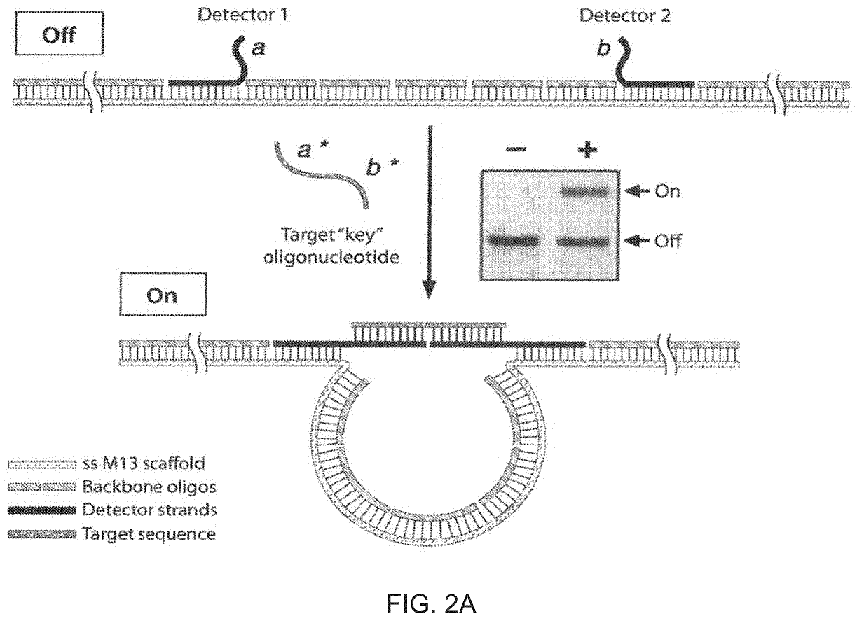

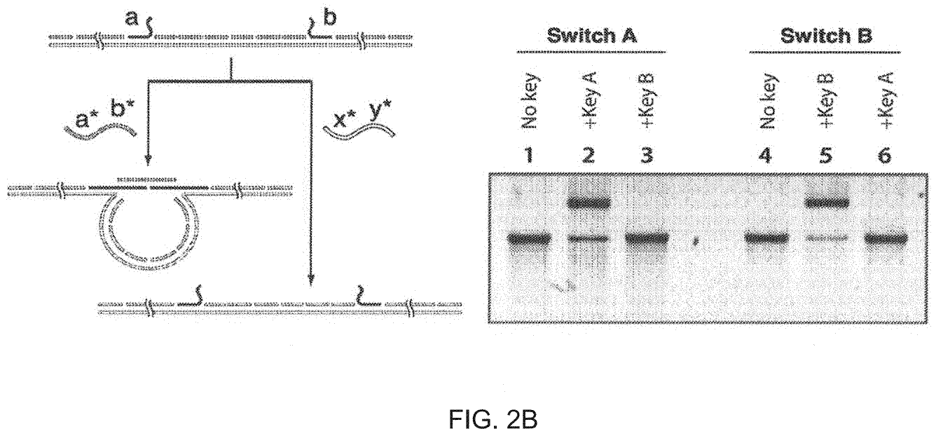

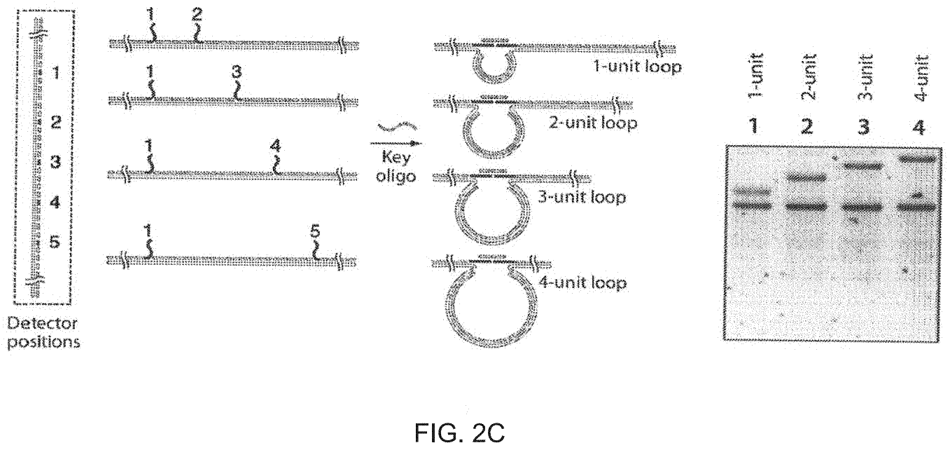

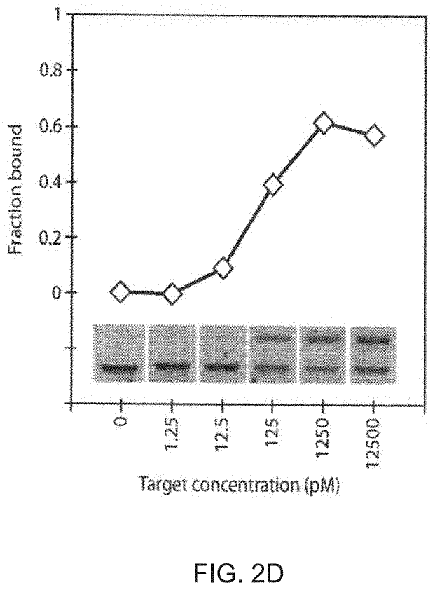

In embodiments, the location of the variable oligonucleotides dictates the location of the various substituents in the complex, such as detector strands, binding partners, latches, etc. It also dictates the size of the loops that are formed once the various substituents bind to each other, as shown in FIGS. 2A and 2C. This will in turn dictate the migration distance of the looped (closed) complex, and thus the ability of the end user to physically separate and thus distinguish between complexes of interest (e.g., closed complexes) and those not of interest (e.g., open complexes).

In embodiments, a nanoswitch may include a first and a second oligonucleotide that together hybridize to a target nucleic acid. In these embodiments, the hybridization of the nanoswitch to the target nucleic acid is considered the first binding interaction. Alternatively, a second binding interaction may be an additional binding interaction that occurs upon hybridization of a second target nucleic acid.

In embodiments, the nanoswitch is designed to detect one target nucleic acid by hybridization of that target to a first oligonucleotide and a second oligonucleotide, each having an overhang (i.e., a single-stranded region that is available for hybridization to the target nucleic acid). Such overhangs are shown in FIG. 2A. The first and second oligonucleotides, in this example, may be referred to as partially hybridized to the scaffold since each has a single-stranded overhang region and a region that is hybridized to the scaffold. The first and second oligonucleotides are denoted “detector 1” and “detector 2”. The overhangs may be referred to herein as 3′ overhangs and 5′ overhangs, referring to the directionality of the single-stranded region. The distance between the first and the second oligonucleotides, when bound to the scaffold, dictates the size of the loop and ultimately the migration distance of the nanoswitch when it is bound to the target (or when it is stabilized) via a latch binding interaction. In embodiments, the detector length may have a length of 5 to 30 or 7-20 nucleotides.

In some embodiments, the first oligonucleotide and the second oligonucleotide are separated from each other by 100-6000 nucleotides. In some instances, the first oligonucleotide and the second oligonucleotide are separated from each other by 500 to 5000 nucleotides, 600-5000 nucleotides, 1000-5000 nucleotides, or 1000-3000 nucleotides. In some embodiments, the first oligonucleotide and the second oligonucleotide are separated from each other by at least 500, 600, 700, 800, 900, 1000, 1500, 2000, 2500, 3000, 3500, 4000, 4500, 5000, 5500, 6000, or more nucleotides. In some embodiments, the first oligonucleotide and the second oligonucleotide are located about equidistant about the center of the scaffold nucleic acid. In some embodiments, the first and second oligonucleotides bind to regions of the scaffold nucleic acid that are internal to the scaffold (i.e., such regions exclude the most 5′ and the most 3′ nucleotides of the scaffold).

### Gel Electrophoresis

In embodiments, such as when measured using gel electrophoresis, the open and closed nanoswitch conformations migrate differentially through a medium such as a gel medium. In embodiments, a circular scaffold such as circular M13 migrates the slowest, a linearized double-stranded version of M13 (without internal binding interactions) migrates fastest, and nanoswitches in looped conformations migrate in between. In embodiments, the migration distance differs based on the length of the loop. As an example, loops that are on the order of about 2590 base pairs are clearly distinguishable from loops that are on the order of about 600 base pairs. Loops of other sizes can also be distinguished from each other, as described herein, and as demonstrated for example in FIG. 2C. The ability to distinguish between loops of different sizes means that the presence (or absence) of multiple targets (each detected by a complex having a loop of a particular size) can be determined simultaneously in a multiplexed assay. Such methods may be used to detect the presence of a single or multiple target and may form the basis of a diagnostic assay. Moreover, it should also be understood that nanoswitches having one loop can also be distinguished from nanoswitches having more than one loop, including those that have 2, 3 or more loops. In embodiments, a single type of nanoswitch can be used to detect two different targets and depending on the conformation of the nanoswitch (as determined by its migration distance in a gel), an end user can determine whether either or both targets are present in a sample. These nanoswitches can then also be extracted from the gel and the bound targets can be isolated.

In embodiments, electrophoresis is performed wherein a gel is run at 4 degrees Celsius to maintain the interaction of the targets to their binding partners (e.g., the binding of a protein target to target-specific antibodies) or to maintain latch binding interactions. It is contemplated that other separation medium is suitable for use herein such those used in capillary electrophoresis and liquid chromatography.

### Nucleic Acid Detection Nanoswitches

In some embodiments, nanoswitches designed for nucleic acid detection are provided. Such nanoswitches comprise a scaffold nucleic acid hybridized to a plurality of oligonucleotides, as described herein. A portion of an exemplary nanoswitch is provided in FIG. 1A. As illustrated, the nanoswitch includes a first and a second oligonucleotide that are partially hybridized to the scaffold nucleic acid (e.g., each of these oligonucleotides is partially hybridized to the scaffold and thus each is partially single-stranded). The first oligonucleotide includes a 3′ overhang and the second oligonucleotide includes a 5′ overhang.

In embodiments, the 3′ overhang is not complementary to the 5′ overhang, and rather both the 3′ and the 5′ overhangs are complementary to a target nucleic acid. FIG. 2A illustrates an embodiment in which the entire target nucleic acid (referred to in the Figure as “Target “Key” oligonucleotide”) hybridizes to a combination of the 3′ and 5′ overhang. However, in embodiments, the method can also be performed in which the 3′ and 5′ overhangs are designed to hybridize only the 5′ and 3′ regions of a target nucleic acid, with the internal or middle region of the target nucleic acid remaining unhybridized. In this latter instance, the nanoswitch is designed to detect a plurality of target nucleic acids of differing sequences provided that they are at least complementary to the 3′ and 5′ overhangs. In embodiments, the nanoswitch detects non-adjacent sequences on the target. Such non-adjacent sequences may be separated by 1 or 2 nucleotides or by 10's or 100's of nucleotides, without limitation. As shown in FIG. 3C shows a portion of the target nucleic acid hybridized to the nanoswitch.

In embodiments, the nanoswitch is configured such that the 3′ and 5′ overhangs come into sufficient proximity to each other in the presence of the target nucleic acid, and that it is only once the target nucleic acid hybridizes to the 3′ and 5′ overhangs that a looped conformation is formed.

In some embodiments, the overhangs may be of different or identical lengths, relative to each other. The overhang length may range from 5-20 nucleotides in length, without limitation. The overhangs may have a length of 5 or more, or 6 or more, or 7 or more nucleotides. One or both overhangs may have a length of 7, 8, 9, 10, 11, 12, 13, 14, 15, 16, 17, 18, 19 or 20 nucleotides, or, in embodiments, include of or consist of a segment having a length of 7, 8, 9, 10, 11, 12, 13, 14, 15, 16, 17, 18, 19 or 20 nucleotides.

In embodiments, the combined length of the overhangs may vary and may depend on their sequence and the length of the target nucleic acid. Their combined length may be 14 nucleotides or longer, without limitation. In some instances, the 3′ overhang and the 5′ overhang are of different lengths and their combined length is at least about 22 nucleotides.

In some embodiments, the combined length of the overhangs may be the same length as the target. Alternatively, the combined length of the overhangs may be longer or shorter than the length of the target. In some embodiments, the target may not bind to both overhangs to the same extent. In other words, one overhang may share more sequence complementarity with the target than the other overhang. In some embodiments, the overhangs will be referred to herein as the 3′ and 5′ overhangs intending the directionality of the overhangs. In some embodiments, the overhangs will be ligated to each other, as described herein, and thus the 3′ overhang may comprise a 3′ hydroxyl and the 5′ overhang may comprise a 5′ phosphate.

In some embodiments, the overhangs may be designed such that they include secondary structure such as but not limited to hairpin conformations. Such secondary structures may be melted during hybridization to the target, or they may be melted as a result of a change in condition or contact with an extrinsic trigger. Thus, also provided herein are compositions comprising any of the foregoing nucleic acid complexes. The composition may comprise a plurality of nucleic acid complexes. The nucleic acid complexes in the plurality may be identical to each other.

Alternatively, the nucleic acid complexes in the plurality may be different from each other. The nanoswitches may differ from each other with respect to their target specificity (e.g., the nucleotide sequence of their 3′ overhangs and/or the sequence of the 5′ overhangs). Nanoswitches may also differ from each other with respect to the distance between the 3′ overhang and the 5′ overhang along the length of the scaffold nucleic acid.

In embodiments, compositions including nanoswitches may further include a sample such as a nucleic acid sample. The sample may or may not comprise the target nucleic acid(s). The composition may or may not comprise the target nucleic acid.

Referring now to FIG. 2B a schematic illustration shows the sequence specificity of embodiments of one or more DNA nanoswitches in accordance with the present disclosure. For example, an agarose gel shows the sequence specificity of the nanoswitch. Switch A turns on only in the presence of key oligonucleotide A and switch B turns on only in the presence of key oligonucleotide B with no background detection of the incorrect strand.

### Target Nucleic Acid

The target nucleic acid may be a DNA, RNA or a combination thereof. It may be a naturally occurring nucleic acid. Examples include an miRNA, a tumor-specific nucleic acid, an allelic variant, and the like, without limitation.

The target nucleic acid, as used herein, refers to the nucleic acid that is hybridized to the nanoswitch. It is to be understood that the target may derive from and thus be a fragment of a much larger nucleic acid such as for example genomic DNA or an mRNA. Thus, a binding portion of the target (e.g., the nucleic acid bound to the nanoswitch) may range from about 7-50 nucleotides, or e.g., 10 to 35 nucleotides, or 5 to 30 nucleotides in some instances, while its parent nucleic acid may be much longer (for example on the order to kbs or more).

In embodiments, the target nucleic acid may be present and thus provided in a nucleic acid sample. The nucleic acid sample is a sample that is being tested for the presence of one or more target nucleic acids. In embodiments, the one or more target nucleic acids may have a known or predetermined nucleic acid sequence, or be substantially similar to a known or predetermined nucleic acid sequence.

In embodiments, the sample may contain the target(s) or it may be suspected of containing the target(s). The sample may comprise non-target nucleic acid. Non-target nucleic acid, as used herein, refers to nucleic acids that are not the targets of interest or do not include a binding portion to the nanoswitch. The methods provided herein allow for the detection of a target nucleic acid even if such target is present in an molar excess of non-target nucleic acid. Thus, the sample may comprise on the order of micromolar quantities of non-target nucleic acid and only nanomolar or picomolar quantities of target nucleic acid and still be able to detect the target. The target nucleic acid and non-target nucleic acid may be present in the sample at a molar ratio of 1:10², 1:10³, 1:10⁴, 1:10⁵, or up to 1:10⁹. The Examples demonstrate detection of picogram quantities of target nucleic acid in the presence of about 100 μM total nucleic acids, the vast majority of which will be non-target nucleic acids.

In some embodiments, the nucleic acid sample may be or may derived from a biological sample such as a bodily fluid (e.g., a blood sample, a urine sample, a sputum sample, a stool sample, a biopsy, and the like). The disclosure contemplates that such samples may be manipulated prior to contact with the nanoswitches. For example, the samples may be treated to lyse cells, degrade or remove protein components, fragment nucleic acids such as genomic DNA, and the like.

In some instances, the target nucleic acid is or is derived from or is a fragment of a miRNA, an mRNA, a genomic DNA, a non-coding RNA, and the like.

In some embodiments, the target-of-interest comprises or consist of ribonucleic acid. In some embodiments, the ribonucleic acid is a messenger RNA (mRNA), a catalytic ribozyme, a self-splicing RNA, a gene regulating RNA, a microRNA, a ribosomal RNA, a viral RNA, a long noncoding RNA, or a chemically modified RNA.

In some embodiments, targets-of-interest include one or more peptide nucleic acids.

In embodiments, the methods of the present disclosure include detection of nucleic acids. Such methods may be used to diagnose a condition, and thus may be referred to herein as diagnostic methods.

In embodiments, a method of the present disclosure includes contacting any of the foregoing nanoswitches with a nucleic acid sample under conditions that allow a target nucleic acid, if present in the nucleic acid sample, to hybridize to the 3′ overhang and the 5′ overhang of the nucleic acid complex, to form a DNA nanoswitch-target complex, and detecting the conformation of the nucleic acid complex by moving through a separation medium such as a gel medium. In embodiments, wherein a looped conformation is present within a medium such as an electrophoresis gel medium, the configuration is indicative of the presence of a specific target-of-interest such as a target nucleic acid (e.g., RNA) in the nucleic acid sample. In embodiments, in the absence of the target nucleic acid, the nucleic acid complex or nanoswitch adopts a linear conformation and a DNA nanoswitch-target complex of the present disclosure is not formed.

In embodiments, the conformation of the nucleic acid complex, e.g., nanoswitch may be determined (or detected) using gel electrophoresis or liquid chromatography, or other separation technique. The gel electrophoresis may be a bufferless gel electrophoresis such as the E-Gel®. Agarose Gel Electrophoresis System (Life Technologies). In embodiments, methods may include detection of the target nucleic acid and detection and optionally purification or substantial purification of the target nucleic acid. In some embodiments, the method may also include measuring an absolute or relative amount of target nucleic acid. This can be done for example by measuring the intensity of bands on a gel or of fractions from a liquid chromatography separation.

In some embodiments, the conditions that allow the target nucleic acid such as RNA to hybridize to the 3′ overhang and the 5′ overhang may be standard hybridization conditions as known in the art. Such conditions may include a suitable concentration of salt(s) and optionally a buffer. The condition may also include EDTA in order to preserve the target nucleic acid and the nucleic acid-based nanoswitch.

In some embodiments, the hybridization may be accomplished using a constant annealing temperature. Such constant temperature may range from about 4° C. to 55° C., 15° C. to 30° C., or 20° C. to 30° C., or may be about 25° C. The temperature may be regarded as room temperature (RT). The hybridization may be carried out over a period of hours such as 1, 2, 3, 4, 5 hours or more.

In some embodiments, the hybridization may be accomplished by decreasing the temperature from a temperature at which the target and the overhangs are not hybridized to each other to a temperature at which they are hybridized to each other. This is referred to herein as a temperature ramp or a decreasing annealing temperature. The starting temperature may be about 40-60° C. without limitation. The ending temperature may be about 4-25° C. without limitation. Thus, the temperature ramp may be from about 50° C. to about 4° C. or about 40° C. to about 4° C. The Examples demonstrate a temperature ramp from about 46° C. to about 4° C. The change in temperature is typically carried out over 1-12 hours. Thus, the change in temperature may decrease by about 0.1-1° C. per minute.

Regardless of whether a constant or decreasing annealing temperature is used, the hybridization may also be carried out for much shorter periods of time, for example on the order of 10-30 minutes, provided readout can be achieved. Thus, in some instances, if the method determines if the target is present, then the hybridization period can be short, particularly if the target is present in abundance. If the method is intended to measure the amount of target in the sample, then longer hybridization times may be required. Similarly, if the target is present in low abundance, longer hybridization times may be required, particularly if an amplifying latch mechanism is used.

In some embodiments, only a portion or preselected portion of the target-of-interest hybridizes to the nanoswitch of the present disclosure.

Referring now to FIG. 2D, data is providing relating to a limit of detection of nucleic acid targets. FIG. 2D shows the signal of the on-state at different target DNA concentrations. The gel of the looped and unlooped nanoswitches is shown as the inset. This disclosure contemplates detecting nucleic acid targets that are present in the picomolar range, as illustrated, as well as in the attomolar range.

In embodiments, the nanoswitches are robust, yielding reproducible results at a variety of DNA and RNA target concentrations including but not limited to 0.25 nM up to 25 nM.

Referring back to FIG. 1, method 100 may start at process sequence 110 by isolating a DNA nanoswitch-target complex within a gel medium such as an electrophoresis gel described above, wherein the DNA nanoswitch-target complex includes a DNA nanoswitch such as described above, and a target-of-interest such as described above. In embodiments, the target-of-interest is RNA. In embodiments, the DNA nanoswitch-target complex includes a DNA nanoswitch such as described above, and a target-of-interest hybridized to the DNA nanoswitch such as described above. In embodiments, the DNA nanoswitch-target complex is in a closed loop configuration.

In embodiments, the nanoswitches can be used to purify targets such as but not limited to target nucleic acids or target proteins from a sample. The looped conformation nanoswitches, which are bound to such targets, can be physically separated using gel electrophoresis from linear conformation nanoswitches, which are not bound to targets. The looped conformation nanoswitches therefore may be physically separated from a complex mixture, and the targets bound thereto can be isolated from the nanoswitches.

### Sequence-Specific RNA Purification.

In embodiments, the closed and open conformations can be separated using gel electrophoresis. Specific RNA targets will only be present in the looped conformation nanoswitches, and these looped conformation nanoswitches, e.g a DNA nanoswitch-target complex can be isolated by gel extraction from electrophoresis forming a DNA nanoswitch-target complex within a gel medium.

In some embodiments, isolation of one or more RNA-looped nanoswitches, e.g. a DNA nanoswitch-RNA target complex may be performed by gel electrophoresis and gel excision, forming a DNA nanoswitch-RNA target complex within a gel medium.

Referring now to FIG. 1 once the DNA nanoswitch-target complex (e.g. looped conformation nanoswitche(s) are isolated and within a gel medium, process sequence 120 includes digesting the DNA nanoswitch and the gel medium to form digested byproducts, and a free target-of-interest. In embodiments, both the nanoswitch and the gel are simultaneously removed for example by digestion using a DNA digesting enzyme such as a nuclease, e.g., DNAse I. In embodiments, both the nanoswitch and the gel are sequentially removed for example by digestion using a DNA digesting enzyme such as a nuclease, e.g., DNAse I, immediately followed by gel removal as described herein. In embodiments, the free target-of-interest is the target in a form unassociated to any additional nucleic acids or byproducts in the reaction medium. In embodiments, free target-of-interest is not hybridized to the DNA nanoswitch.

Referring now to FIG. 1, at process sequence 130, method 100 includes isolating the free target-of-interest. In embodiments, the free target-of-interest is a single RNA species. Thus, following digestion of the DNA and the gel, the target-of-interest such as RNA can be further purified using byproducts using e.g., a commercially available kit to dissolve the agarose gel pieces and then to purify the RNA from enzymes and nanoswitch fragments removing digested byproducts, or the other components of the solution. Using this method, it is possible to isolate and purify a single RNA sequence from complex mixtures for various downstream applications. In instances in which the overhangs of the nanoswitch hybridize to the target only partially, then it is contemplated the nanoswitches may capture a plurality of targets, all of which will have identical sequences at their 5′ and 3′ ends (as a result of being hybridized and thus captured by the same overhangs) but which will differ from each other in their internal sequence between such ends.

In embodiments, the present disclosure relates to a detect-and-purify method for single species RNA purification (FIGS. 3A-3D) that overcomes many drawbacks of current approaches. Instead of capturing RNAs on a solid support (e.g. a magnetic bead), the present disclosure provides DNA nanoswitches that change conformations upon binding the targeted sequence. In embodiments, the nanoswitch is a linear double stranded DNA (dsDNA) with two ssDNA capture probes (oligo sequences presented. Non-limiting examples of nucleic acids such as DNA suitable for use herein is disclosed in Tables below such as Tables 1 to 5. In embodiments, target hybridization to a DNA nanoswitch causes the DNA nanoswitch to reconfigure to a looped dsDNA (FIG. 3B). See e.g., Chandrasekaran, A. R. et al. Cellular microRNA detection with miRacles: microRNA-activated conditional looping of engineered switches. Science Advances 5, eaau9443 (2019).

In embodiments, the methods of the present disclosure include: 1) isolation of the RNA-looped nanoswitches by gel electrophoresis and gel excision, 2) digestion of DNA nanoswitches and gel pieces, and 3) removal of digested byproducts. In embodiments, the DNA nanoswitches capture target RNA molecules and become looped. Looped nanoswitches are separated and imaged using gel electrophoresis and isolated using gel excision. The nanoswitches are then digested using nuclease such as DNase I, which was found to work even on intact gel pieces. In embodiments, the process sequence removes byproducts using a commercially available kit to dissolve the agarose gel pieces and then to purify the RNA from enzymes and nanoswitch fragments (FIG. 3C).

### Kits

In embodiments, the present disclosure further provides a kit including a single-stranded scaffold nucleic acid, and a plurality of single-stranded oligonucleotides, each having a sequence complementary to a sequence on the scaffold nucleic acid, wherein when the oligonucleotides are hybridized to the scaffold nucleic acid no overlap exists between the oligonucleotides. In some instances, each oligonucleotide, in this first subset of oligonucleotides, has a sequence that is complementary to a contiguous sequence on the scaffold nucleic acid intending that every nucleotide in the oligonucleotide is hybridized with a nucleotide in the scaffold, and no “single-stranded bubbles” exist following hybridization.

In some embodiments, the kit further includes, in some instances, a subset of oligonucleotides, for example two, four, six or more oligonucleotides, that are either detector oligonucleotides such as those shown in FIG. 2A and/or are modified oligonucleotides. The subset of oligonucleotides may comprise for example a first and a second oligonucleotide that each comprise a nucleotide sequence that is complementary to a target nucleic acid. In this manner, the kit is intended to be used to detect a target nucleic acid of known or at least partially known sequence. Such target nucleic acid may be an allelic variant of genomic locus, or a cancer-specific nucleic acid such as may be found circulating in the blood of a subject having cancer, or a miRNA, without limitation. The subset of oligonucleotides may additionally comprise a third and a fourth oligonucleotide that each comprise a nucleotide sequence that is complementary to a second target nucleic acid.

The subset of oligonucleotides may additionally include a pair of oligonucleotides that each comprise a nucleotide sequence complementary to a trigger (or latch) nucleic acid. The trigger (or latch) nucleic acid is also included in the kit, in such instances.

In embodiments, a kit includes scaffold DNA in an amount sufficient to provide about 0.001 to 0.003 micrograms per gel lane; oligonucleotides in an amount sufficient to provide about 0.001 to 0.003 micrograms per gel lane; linearization enzyme in an amount sufficient to provide 0.002 to 0.004. micrograms per gel lane. In embodiments, reagents are provided such as agarose, GelRed, and loading dye. Agarose may be provided in an amount of about 0.01 to 0.03 g. In embodiments, GelRed may be provided in an amount of about 0.0001 microliters. In embodiments loading dye such as FICOLL may be provided in an amount of 0.0002 to 0.0004 g.

In some embodiments, the present disclosure relates to a method of purifying a single ribonucleic acid (RNA) species, including: isolating a DNA nanoswitch-target complex within a gel medium, wherein the DNA nanoswitch-target complex includes a DNA nanoswitch and a target-of-interest; digesting the DNA nanoswitch and the gel medium to form digested byproducts, and a free target-of-interest; and isolating the free target-of-interest, wherein the free target-of-interest is a single RNA species. In some embodiments, the free target-of-interest includes or consist of ribonucleic acid. In some embodiments, the ribonucleic acid is a messenger RNA (mRNA), a catalytic ribozyme, a self-splicing RNA, or a gene regulating RNA. In some embodiments, isolating a DNA nanoswitch-target complex within a gel medium includes electrophoresing the DNA nanoswitch-target complex in an electrophoresis gel and excising the DNA nanoswitch-target complex. In some embodiments, the DNA nanoswitch is characterized as looped. In some embodiments, digesting the DNA nanoswitch and the gel medium comprises contacting the DNA nanoswitch and the gel medium with DNase. In some embodiments, the method further includes, prior to isolating a DNA nanoswitch-target complex, contacting a preselected deoxyribonucleic acid (DNA) nanoswitch and a target to form a DNA nanoswitch-target complex. In embodiments, isolating includes dissolving the digested byproducts and purifying the free target-of-interest. In embodiments, the digested byproducts comprise agarose gel pieces, enzyme, and nanoswitch fragments.

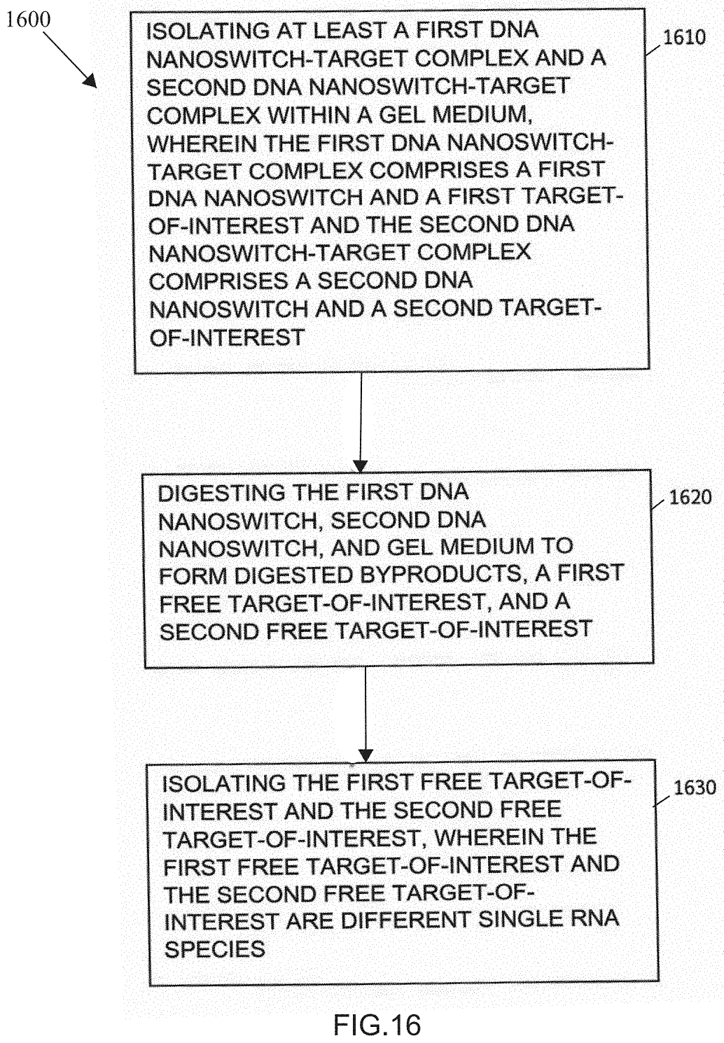

Referring now to FIG. 16, the present disclosure includes method 1600. In embodiments, method 1600 includes at process sequence 1610 isolating at least a first DNA nanoswitch-target complex and a second DNA nanoswitch-target complex within a gel medium, wherein the first DNA nanoswitch-target complex includes a first DNA nanoswitch and a first target-of-interest and the second DNA nanoswitch-target complex comprises a second DNA nanoswitch and a second target-of-interest. At process sequence 1620, method 1600 includes digesting the first DNA nanoswitch, second DNA nanoswitch, and gel medium to form digested byproducts, a first free target-of-interest, and a second free target-of-interest. At process sequence 1630, method 1600 includes isolating the first free target-of-interest and the second free target-of-interest, wherein the first free target-of-interest and the second free target-of-interest are different single RNA species. In some embodiments, the first free target-of-interest and second free target-of-interest comprise or consist of ribonucleic acid. In some embodiments, the ribonucleic acid is messenger RNA (mRNA), catalytic ribozyme, self-splicing RNA, a gene regulating RNA, a microRNA, ribosomal RNA, or viral RNA.

In some embodiments, the first DNA nanoswitch and second DNA nanoswitch are each characterized as looped. In some embodiments, isolating includes electrophoresing the first and second nanoswitch-target complexes in an electrophoresis gel and excising the first and second nanoswitch-target complexes. In some embodiments, digesting includes contacting the first DNA nanoswitch, second DNA nanoswitch, and gel medium with a nuclease such as DNase. In some embodiments, the DNAse is DNase I. In some embodiments, isolating includes dissolving the digested byproducts and purifying the first free target-of-interest and the second free target-of-interest. In some embodiments, the digested byproducts comprise agarose gel pieces, enzyme, and nanoswitch fragments.

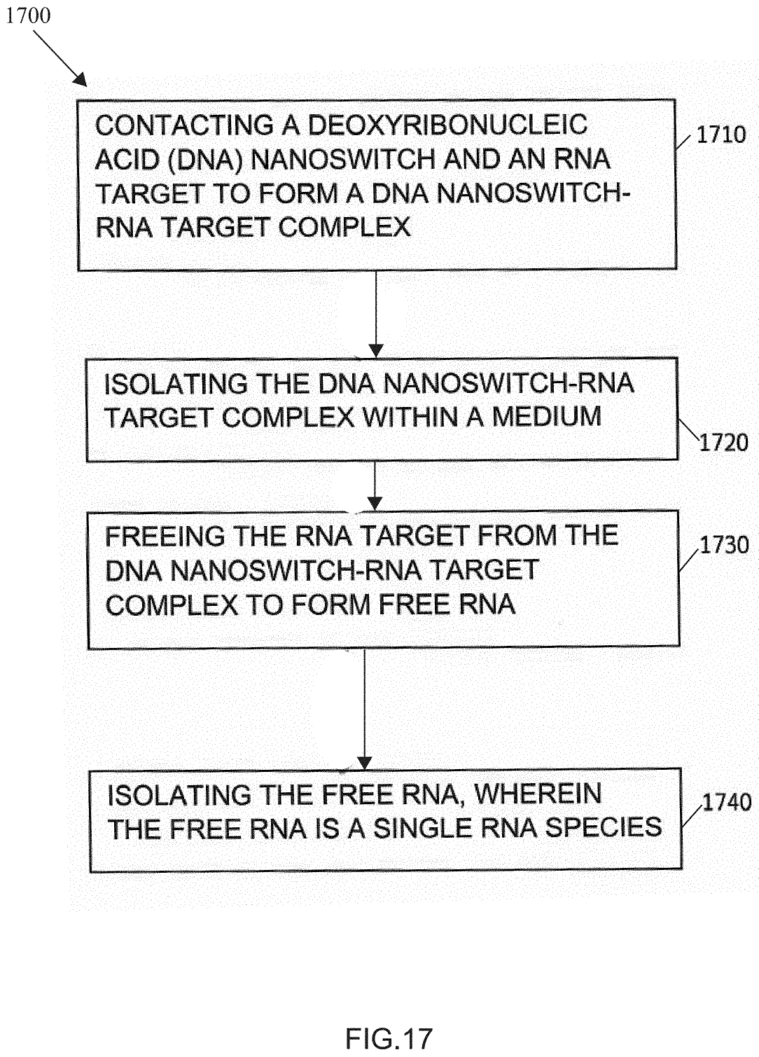

Referring now to FIG. 17, the present disclosure includes method 1700 of purifying a single ribonucleic acid (RNA) species. In embodiments, method 1700 includes at process sequence 1710 contacting a deoxyribonucleic acid (DNA) nanoswitch and an RNA target to form a DNA nanoswitch-RNA target complex. In embodiments, method 1700 includes at process sequence 1720 isolating the DNA nanoswitch-RNA target complex within a medium. In embodiments, method 1700 includes at process sequence 1730 freeing the RNA target from the DNA nanoswitch-RNA target complex to form free RNA. In embodiments, method 1700 includes at process sequence 1740 isolating the free RNA, wherein the free RNA is a single RNA species. In embodiments, the single RNA species is characterized as substantially purified. In some embodiments, the free RNA is a messenger RNA (mRNA), a catalytic ribozyme, a self-splicing RNA, or a gene regulating RNA.

The following Tables are referred to herein including:

TABLE 1
Table 1 relating to the sequence of target mRNA fragment and oligos used
for the design of nanoswitch embodiments.

| Name | Sequence (5′→3′) | Len. |
|---|---|---|
| mRNA | CUGGACCUCCCAAAAGCCAACUUAUUGUGAUAUUUGUAAA | 401 |
| fragment | UUAUAGUUUUAGCAGUUCGUUUGCCACAUGAGUGGAACA | |
| | UCGUGAAUGCACUUUUGAUAAGUGCUCGGUUAUUUUAUA | |
| | UUGUAACUACCAGCCUUCAGAGGCGAUCGUAUGCAUAGU | |
| | UUCUUGAAGUCAAUUUGUCCGUGUAUUCAAAUGUUUGCU | |
| | UUCGUGAAAACUCGCAUUGUUUUGUCACUCUACCAAGUAA | |
| | UCAAUUUGUACCAAUCAAUCGCAUAUGGUUGUCCUAGAUC | |
| | UAAAAAUGGCAAUAAUUUGCCUUCGGUAUUGCACCUAAUG | |
| | UAUUCAAGAACAAGUAGGGAAGCUCGAAAUUUCUCAAAU | |
| | ACUUACCCAAAAAAUAGAUAGAAAUAUAUUUUCGAUUCG | |
| | CAAUCGU (SEQ ID NO: 1) | |
| mRNA | AAGCTCGAAATTTCTCAAATACTTACCCAAAAAATAGATA | 40 |
| frag_DNA | (SEQ ID NO: 2) | |
| Target | | |
| mRNA_Probe1_ | ACCGTTGTAGCAATACTTCTTTGATTAGTAATAACATCACTA | 60 |
| 20 nt | TCTATTTTTTGGGTAAGT (SEQ ID NO: 3) | |
| mRNA_Probe2_ | ATTTGAGAAATTTCGAGCTTTCAACCGATTGAGGGAGGGAA | 60 |
| 20 nt_big | GGTAAATATTGACGGAAAT (SEQ ID NO: 4) | |
| loop | | |
| mRNA_Probe1_ | TGTAGCAATACTTCTTTGATTAGTAATAACATCACATTTTTTG | 55 |
| 15 nt | GGTAAGTCAGTC (SEQ ID NO: 5) | |
| mRNA_Probe2_ | GTCAGATTTGAGAAATTTCGTCAACCGATTGAGGGAGGGA | 55 |
| 15 nt_big | AGGTAAATATTGACG (SEQ ID NO: 6) | |
| loop | | |
| mRNA_Probe1_ | TGTAGCAATACTTCTTTGATTAGTAATAACATCACTTTTGGG | 52 |
| 12 nt | TAAGTCAGTC (SEQ ID NO: 7) | |
| mRNA_Probe2_ | GTCAGATTTGAGAAATTTCAACCGATTGAGGGAGGGAAGG | 52 |
| 12 nt_big | TAAATATTGACG_(SEQ ID NO: 8) | |
| loop | | |
| mRNA_Probe1 _ | TGTAGCAATACTTCTTTGATTAGTAATAACATCACGGGTAAG | 48 |
| 8nt | TCAGTC (SEQ ID NO: 9) | |
| mRNA_Probe2_ | GTCAGATTTGAGATCAACCGATTGAGGGAGGGAAGGTAAA | 48 |
| 8 nt_big | TATTGACG (SEQ ID NO: 10) | |
| loop | | |
| mRNA_Probe2_ | ATTTGAGAAATTTCGAGCTTTGGGTTATATAACTATATGTAA | 60 |
| small loop | ATGCTGATGCAAATCCAA (SEQ ID NO: 11) | |

It is contemplated that certain alterations can be made to the above-referenced sequences, thus sequences suitable for use herein may, in embodiments, included nucleic acid sequences including or consisting of amino acid sequences having at least 80%, 90%, 95%, 97%, and 99% sequence identity to the sequences listed above in Table 1.

TABLE 2
Sequence of target miR-206 and oligos used for the design of
nanoswitches.

| Name | Sequence (5′→3′) | Len. |
|---|---|---|
| miR-206 | UGGAAUGUAAGGAAGUGUGUGG (SEQ ID NO: 12) | 22 |
| miR-206 | TGGAATGTAAGGAAGTGTGTGG (SEQ ID NO: 13) | 22 |
| DNA Target | | |
| miR206_ | ACCGTTGTAGCAATACTTCTTTGATTAGTAATAACATCACCC | 51 |
| Probe1 | ACACACTTC (SEQ ID NO: 14) | |
| miR206_ | CTTACATTCCATCAACCGATTGAGGGAGGGAAGGTAAATAT | 51 |
| Probe2_big | TGACGGAAAT (SEQ ID NO: 15) | |
| loop | | |

It is contemplated that certain alterations can be made to the above-referenced sequences, thus sequences suitable for use herein may, in embodiments, included nucleic acid sequences including or consisting of amino acid sequences having at least 80%, 90%, 95%, 97%, and 99% sequence identity to the sequences listed above in Table 2.

TABLE 3
Sequences of target ribosomal RNA 5.8S and 5S and oligos used for the
design of nanoswitches.

| Name | Sequence (5′→3′) | Len. |
|---|---|---|
| rRNA 5.8S | CGACUCUUAGCGGUGGAUCACUCGGCUCGUGCGUCGAUG | 22 |
| (NR_146147.1) | AAGAACGCAGCUAGCUGCGAGAAUUAAUGUGAAUUGCAG | |
| | GACACAUUGAUCAUCGACACUUCGAACGCACUUGCGGCC | |
| | CCGGGUUCCUCCCGGGGCUACGCCUGUCUGAGCGUCGC | |
| | UU (SEQ ID NO: 16) | |
| 5.8S_DNA | UGGAAUGUAAGGAAGUGUGUGG (SEQ ID NO: 17) | 157 |
| Target | | |
| 5.8S_Probe1 | ACCGTTGTAGCAATACTTCTTTGATTAGTAATAACATCACTC | 55 |
| | ATCGACGCACGAG (SEQ ID NO: 18) | |
| 5.8S_Probe2_ | CCGAGTGATCCACCGTCAACCGATTGAGGGAGGGAAGGTA | 55 |
| big loop | AATATTGACGGAAAT (SEQ ID NO: 19) | |
| rRNA 5S | GUCUACGGCCAUACCACCCUGAACGCGCCCGAUCUCGUC | 121 |
| (NR_023376.1) | UGAUCUCGGAAGCUAAGCAGGGUCGGGCCUGGUUAGUAC | |
| | UUGGAUGGGAGACCGCCUGGGAAUACCGGGUGCUGUAGG | |
| | CUUU (SEQ ID NO: 20) | |
| 5S_DNA | TAGTACTTGGATGGGAGACCGCCTGGGAAT (SEQ ID NO: | 30 |
| Target | 21) | |
| 5S_Probe1 | ACCGTTGTAGCAATACTTCTTTGATTAGTAATAACATCACAT | 55 |
| | TCCCAGGCGGTCT (SEQ ID NO: 22) | |
| 5S_Probe2_ | CCCATCCAAGTACTATGGGTTATATAACTATATGTAAATGCT | 55 |
| big loop | GATGCAAATCCAA (SEQ ID NO: 23) | |

It is contemplated that certain alterations can be made to the above-referenced sequences, thus sequences suitable for use herein may, in embodiments, included nucleic acid sequences including or consisting of amino acid sequences having at least 80%, 90%, 95%, 97%, and 99% sequence identity to the sequences listed above in Table 3.

TABLE 4
Sequence of synthesized RNA with m^{4,4}C chemical modification and
oligos usedf or the design of nanoswitches.

| Name | Sequence (5′→3′) | Len. |
|---|---|---|
| RNA_m^{4,4}C | UAGUCUGCACCUGCACCAGUCGCUCAGGGAU (SEQ ID NO: | 31 |
| | 24) | |
| RNA_m^{4,4}C_ | TAGTCTGCACCTGCACCAGTCGCTCAGGGAT (SEQ ID NO: | 31 |
| DNA Target | 25) | |
| NS1_Probe1 | ACCGTTGTAGCAATACTTCTTTGATTAGTAATAACATCACAT | 50 |
| | CCCTGAGC (SEQ ID NO: 26) | |
| NS1_Probe2_ | GACTGGTGCATCAACCGATTGAGGGAGGGAAGGTAAATAT | 50 |
| big loop | TGACGGAAAT (SEQ ID NO: 27) | |
| NS2_Probe1 | ACCGTTGTAGCAATACTTCTTTGATTAGTAATAACATCACTC | 55 |
| | CCTGAGCGACTGG (SEQ ID NO: 28) | |
| NS2_Probe2_ | TGCAGGTGCAGACTATCAACCGATTGAGGGAGGGAAGGTA | 55 |
| big loop | AATATTGACGGAAAT (SEQ ID NO: 29) | |

It is contemplated that certain alterations can be made to the above-referenced sequences, thus sequences suitable for use herein may, in embodiments, included nucleic acid sequences including or consisting of amino acid sequences having at least 80%, 90%, 95%, 97%, and 99% sequence identity to the sequences listed above in Table 4.

TABLE 5
Backbone used for the design of nanoswitches and blocking oligo used
in some of the detection assays^{1,2}.
Backbone oligos

| # | Sequence (5′-3′) | Length |
|---|---|---|
| 1 | AGAGCATAAAGCTAAATCGGTTGTACCAAAAACATTATGACCCTGTA | 60 |
| | ATACTTTTGCGGG (SEQ ID NO: 30) | |
| 2 | AGAAGCCTTTATTTCAACGCAAGGATAAAAATTTTTAGAACCCTCATA | 60 |
| | TATTTTAAATGC (SEQ ID NO: 31) | |
| 3 | AATGCCTGAGTAATGTGTAGGTAAAGATTCAAAAGGGTGAGAAAGG | 60 |
| | CCGGAGACAGTCAA (SEQ ID NO: 32) | |
| 4 | ATCACCATCAATATGATATTCAACCGTTCTAGCTGATAAATTAATGCC | 60 |
| | GGAGAGGGTAGC (SEQ ID NO: 33) | |
| 5 | TATTTTTGAGAGATCTACAAAGGCTATCAGGTCATTGCCTGAGAGTC | 60 |
| | TGGAGCAAACAAG (SEQ ID NO: 34) | |
| 6 | AGAATCGATGAACGGTAATCGTAAAACTAGCATGTCAATCATATGTA | 60 |
| | CCCCGGTTGATAA (SEQ ID NO: 35) | |
| 7 | TCAGAAAAGCCCCAAAAACAGGAAGATTGTATAAGCAAATATTTAAA | 60 |
| | TTGTAAACGTTAA (SEQ ID NO: 36) | |
| 8 | TATTTTGTTAAAATTCGCATTAAATTTTTGTTAAATCAGCTCATTTTTT | 60 |
| | AACCAATAGGA (SEQ ID NO: 37) | |
| 9 | ACGCCATCAAAAATAATTCGCGTCTGGCCTTCCTGTAGCCAGCTTTC | 60 |
| | ATCAACATTAAAT (SEQ ID NO: 38) | |
| 10 | GGATAGGTCACGTTGGTGTAGATGGGCGCATCGTAACCGTGCATCT | 60 |
| | GCCAGTTTGAGGGG (SEQ ID NO: 39) | |
| 11 | ACGACGACAGTATCGGCCTCAGGAAGATCGCACTCCAGCCAGCTTT | 60 |
| | CCGGCACCGCTTCT (SEQ ID NO: 40) | |
| 12 | GGTGCCGGAAACCAGGCAAAGCGCCATTCGCCATTCAGGCTGCGC | 60 |
| | AACTGTTGGGAAGGG (SEQ ID NO: 41) | |
| 13 | CGATCGGTGCGGGCCTCTTCGCTATTACGCCAGCTGGCGAAAGGG | 60 |
| | GGATGTGCTGCAAGG (SEQ ID NO: 42) | |
| 14 | CGATTAAGTTGGGTAACGCCAGGGTTTTCCCAGTCACGACGTTGTA | 60 |
| | AAACGACGGCCAGT (SEQ ID NO: 43) | |
| 15 | GCCAAGCTTGCATGCCTGCAGGTCGACTCTAGAGGATCCCCGGGT | 60 |
| | ACCGAGCTCGAATTC (SEQ ID NO: 44) | |
| 16 | GTAATCATGGTCATAGCTGTTTCCTGTGTGAAATTGTTATCCGCTCA | 60 |
| | CAATTCCACACAA (SEQ ID NO: 45) | |
| 17 | CATACGAGCCGGAAGCATAAAGTGTAAAGCCTGGGGTGCCTAATGA | 60 |
| | GTGAGCTAACTCAC (SEQ ID NO: 46) | |
| 18 | ATTAATTGCGTTGCGCTCACTGCCCGCTTTCCAGTCGGGAAACCTG | 60 |
| | TCGTGCCAGCTGCA (SEQ ID NO: 47) | |
| 19 | TTAATGAATCGGCCAACGCGCGGGGAGAGGCGGTTTGCGTATTGG | 60 |
| | GCGCCAGGGTGGTTT (SEQ ID NO: 48) | |
| 20 | GTTGCAGCAAGCGGTCCACGCTGGTTTGCCCCAGCAGGCGAAAAT | 60 |
| | CCTGTTTGATGGTGG (SEQ ID NO: 49) | |
| 21 | TTCCGAAATCGGCAAAATCCCTTATAAATCAAAAGAATAGCCCGAGA | 60 |
| | TAGGGTTGAGTGT (SEQ ID NO: 50) | |
| 22 | TGTTCCAGTTTGGAACAAGAGTCCACTATTAAAGAACGTGGACTCCA | 60 |
| | ACGTCAAAGGGCG (SEQ ID NO: 51) | |
| 23 | AAAAACCGTCTATCAGGGCGATGGCCCACTACGTGAACCATCACCC | 60 |
| | AAATCAAGTTTTTT (SEQ ID NO: 52) | |
| 24 | GGGGTCGAGGTGCCGTAAAGCACTAAATCGGAACCCTAAAGGGAG | 60 |
| | CCCCCGATTTAGAGC (SEQ ID NO: 53) | |
| 25 | TTGACGGGGAAAGCCGGCGAACGTGGCGAGAAAGGAAGGGAAGAA | 60 |
| | AGCGAAAGGAGCGGG (SEQ ID NO: 54) | |
| 26 | CGCTAGGGCGCTGGCAAGTGTAGCGGTCACGCTGCGCGTAACCAC | 60 |
| | CACACCCGCCGCGCT (SEQ ID NO: 55) | |
| 27 | TAATGCGCCGCTACAGGGCGCGTACTATGGTTGCTTTGACGAGCAC | 60 |
| | GTATAACGTGCTTT (SEQ ID NO: 56) | |
| 28 | CCTCGTTAGAATCAGAGCGGGAGCTAAACAGGAGGCCGATTAAAGG | 60 |
| | GATTTTAGACAGGA (SEQ ID NO: 57) | |
| 29 | ACGGTACGCCAGAATCCTGAGAAGTGTTTTTATAATCAGTGAGGCC | 60 |
| | ACCGAGTAAAAGAG (SEQ ID NO: 58) | |
| 30 | TTGCCTGAGTAGAAGAACTCAAACTATCGGCCTTGCTGGTAATATCC | 60 |
| | AGAACAATATTAC (SEQ ID NO: 59) | |
| 31 | CGCCAGCCATTGCAACAGGAAAAACGCTCATGGAAATACCTACATTT | 60 |
| | TGACGCTCAATCG (SEQ ID NO: 60) | |
| 32 | TCTGAAATGGATTATTTACATTGGCAGATTCACCAGTCACACGACCA | 60 |
| | GTAATAAAAGGGA (SEQ ID NO: 61) | |
| 33 | CATTCTGGCCAACAGAGATAGAACCCTTCTGACCTGAAAGCGTAAG | 60 |
| | AATACGTGGCACAG (SEQ ID NO: 62) | |
| 34 | ACAATATTTTTGAATGGCTATTAGTCTTTAATGCGCGAACTGATAGC | 60 |
| | CCTAAAACATCGC (SEQ ID NO: 63) | |
| 35 | CATTAAAAATACCGAACGAACCACCAGCAGAAGATAAAACAGAGGT | 60 |
| | GAGGCGGTCAGTAT (SEQ ID NO: 64) | |
| 36 | TAACACCGCCTGCAACAGTGCCACGCTGAGAGCCAGCAGCAAATGA | 60 |
| | AAAATCTAAAGCAT (SEQ ID NO: 65) | |
| 37 | CACCTTGCTGAACCTCAAATATCAAACCCTCAATCAATATCTGGTCA | 60 |
| | GTTGGCAAATCAA (SEQ ID NO: 66) | |
| 38 | CAGTTGAAAGGAATTGAGGAAGGTTATCTAAAATATCTTTAGGAGCA | 60 |
| | CTAACAACTAATA (SEQ ID NO: 67) | |
| 39 | GATTAGAGCCGTCAATAGATAATACATTTGAGGATTTAGAAGTATTA | 60 |
| | GACTTTACAAACA (SEQ ID NO: 68) | |
| 40 | CATTATCATTTTGCGGAACAAAGAAACCACCAGAAGGAGCGGAATTA | 60 |
| | TCATCATATTCCT (SEQ ID NO: 69) | |
| 41 | GATTATCAGATGATGGCAATTCATCAATATAATCCTGATTGTTTGGAT | 60 |
| | TATACTTCTGAA (SEQ ID NO: 70) | |
| 42 | TAATGGAAGGGTTAGAACCTACCATATCAAAATTATTTGCACGTAAA | 60 |
| | ACAGAAATAAAGA (SEQ ID NO: 71) | |
| 43 | AATTGCGTAGATTTTCAGGTTTAACGTCAGATGAATATACAGTAACA | 60 |
| | GTACCTTTTACAT (SEQ ID NO: 72) | |
| 44 | CGGGAGAAACAATAACGGATTCGCCTGATTGCTTTGAATACCAAGTT | 60 |
| | ACAAAATCGCGCA (SEQ ID NO: 73) | |
| 45 | GAGGCGAATTATTCATTTCAATTACCTGAGCAAAAGAAGATGATGAA | 60 |
| | ACAAACATCAAGA (SEQ ID NO: 74) | |
| 46 | AAACAAAATTAATTACATTTAACAATTTCATTTGAATTACCTTTTTTAA | 60 |
| | TGGAAACAGTA (SEQ ID NO: 75) | |
| 47 | CATAAATCAATATATGTGAGTGAATAACCTTGCTTCTGTAAATCGTCG | 60 |
| | CTATTAATTAAT (SEQ ID NO: 76) | |
| 48 | TTTCCCTTAGAATCCTTGAAAACATAGCGATAGCTTAGATTAAGACG | 60 |
| | CTGAGAAGAGTCA (SEQ ID NO: 77) | |
| 49 | ATAGTGAATTTATCAAAATCATAGGTCTGAGAGACTACCTTTTTAACC | 60 |
| | TCCGGCTTAGGT (SEQ ID NO: 78) | |
| 50 | GAAAACTTTTTCAAATATATTTTAGTTAATTTCATCTTCTGACCTAAAT | 60 |
| | TTAATGGTTTG (SEQ ID NO: 79) | |
| 51 | AAATACCGACCGTGTGATAAATAAGGCGTTAAATAAGAATAAACACC | 60 |
| | GGAATCATAATTA (SEQ ID NO: 80) | |
| 52 | CTAGAAAAAGCCTGTTTAGTATCATATGCGTTATACAAATTCTTACCA | 60 |
| | GTATAAAGCCAA (SEQ ID NO: 81) | |
| 53 | CGCTCAACAGTAGGGCTTAATTGAGAATCGCCATATTTAACAACGCC | 60 |
| | AACATGTAATTTA (SEQ ID NO: 82) | |
| 54 | GGCAGAGGCATTTTCGAGCCAGTAATAAGAGAATATAAAGTACCGA | 60 |
| | CAAAAGGTAAAGTA (SEQ ID NO: 83) | |
| 55 | ATTCTGTCCAGACGACGACAATAAACAACATGTTCAGCTAATGCAGA | 60 |
| | ACGCGCCTGTTTA (SEQ ID NO: 84) | |
| 56 | TCAACAATAGATAAGTCCTGAACAAGAAAAATAATATCCCATCCTAAT | 60 |
| | TTACGAGCATGT (SEQ ID NO: 85) | |
| 57 | AGAAACCAATCAATAATCGGCTGTCTTTCCTTATCATTCCAAGAACG | 60 |
| | GGTATTAAACCAA (SEQ ID NO: 86) | |
| 58 | GTACCGCACTCATCGAGAACAAGCAAGCCGTTTTTATTTTCATCGTA | 60 |
| | GGAATCATTACCG (SEQ ID NO: 87) | |
| 59 | CGCCCAATAGCAAGCAAATCAGATATAGAAGGCTTATCCGGTATTCT | 60 |
| | AAGAACGCGAGGC (SEQ ID NO: 88) | |
| 60 | ATTTTGCACCCAGCTACAATTTTATCCTGAATCTTACCAACGCTAAC | 60 |
| | GAGCGTCTTTCCA (SEQ ID NO: 89) | |
| 61 | GAGCCTAATTTGCCAGTTACAAAATAAACAGCCATATTATTTATCCCA | 60 |
| | ATCCAAATAAGA (SEQ ID NO: 90) | |
| 62 | AACGATTTTTTGTTTAACGTCAAAAATGAAAATAGCAGCCTTTACAGA | 60 |
| | GAGAATAACATA (SEQ ID NO: 91) | |
| 63 | AAAACAGGGAAGCGCATTAGACGGGAGAATTAACTGAACACCCTGA | 60 |
| | ACAAAGTCAGAGGG (SEQ ID NO: 92) | |
| 64 | TAATTGAGCGCTAATATCAGAGAGATAACCCACAAGAATTGAGTTAA | 60 |
| | GCCCAATAATAAG (SEQ ID NO: 93) | |
| 65 | AGCAAGAAACAATGAAATAGCAATAGCTATCTTACCGAAGCCCTTTT | 60 |
| | TAAGAAAAGTAAG (SEQ ID NO: 94) | |
| 66 | CAGATAGCCGAACAAAGTTACCAGAAGGAAACCGAGGAAACGCAAT | 60 |
| | AATAACGGAATACC (SEQ ID NO: 95) | |
| 67 | CAAAAGAACTGGCATGATTAAGACTCCTTATTACGCAGTATGTTAGC | 60 |
| | AAACGTAGAAAAT (SEQ ID NO: 96) | |
| 68 | ACATACATAAAGGTGGCAACATATAAAAGAAACGCAAAGACACCAC | 60 |
| | GGAATAAGTTTATT (SEQ ID NO: 97) | |
| 69 | TTGTCACAATCAATAGAAAATTCATATGGTTTACCAGCGCCAAAGAC | 60 |
| | AAAAGGGCGACAT (SEQ ID NO: 98) | |
| 70 | TCACCGTCACCGACTTGAGCCATTTGGGAATTAGAGCCAGCAAAAT | 60 |
| | CACCAGTAGCACCA (SEQ ID NO: 99) | |
| 71 | TTACCATTAGCAAGGCCGGAAACGTCACCAATGAAACCATCGATAG | 60 |
| | CAGCACCGTAATCA (SEQ ID NO: 100) | |
| 72 | GTAGCGACAGAATCAAGTTTGCCTTTAGCGTCAGACTGTAGCGCGT | 60 |
| | TTTCATCGGCATTT (SEQ ID NO: 101) | |
| 73 | TCGGTCATAGCCCCCTTATTAGCGTTTGCCATCTTTTCATAATCAAAA | 60 |
| | TCACCGGAACCA (SEQ ID NO: 102) | |
| 74 | GAGCCACCACCGGAACCGCCTCCCTCAGAGCCGCCACCCTCAGAA | 60 |
| | CCGCCACCCTCAGAG (SEQ ID NO: 103) | |
| 75 | CCACCACCCTCAGAGCCGCCACCAGAACCACCACCAGAGCCGCCG | 60 |
| | CCAGCATTGACAGGA (SEQ ID NO: 104) | |
| 76 | GGTTGAGGCAGGTCAGACGATTGGCCTTGATATTCACAAACAAATA | 60 |
| | AATCCTCATTAAAG (SEQ ID NO: 105) | |
| 77 | CCAGAATGGAAAGCGCAGTCTCTGAATTTACCGTTCCAGTAAGCGT | 60 |
| | CATACATGGCTTTT (SEQ ID NO: 106) | |
| 78 | GATGATACAGGAGTGTACTGGTAATAAGTTTTAACGGGGTCAGTGC | 60 |
| | CTTGAGTAACAGTG (SEQ ID NO: 107) | |
| 79 | CCCGTATAAACAGTTAATGCCCCCTGCCTATTTCGGAACCTATTATT | 60 |
| | CTGAAACATGAAA (SEQ ID NO: 108) | |
| 80 | CCAGGCGGATAAGTGCCGTCGAGAGGGTTGATATAAGTATAGCCCG | 60 |
| | GAATAGGTGTATCA (SEQ ID NO: 109) | |
| 81 | CCGTACTCAGGAGGTTTAGTACCGCCACCCTCAGAACCGCCACCCT | 60 |
| | CAGAACCGCCACCC (SEQ ID NO: 110) | |
| 82 | TCAGAGCCACCACCCTCATTTTCAGGGATAGCAAGCCCAATAGGAA | 60 |
| | CCCATGTACCGTAA (SEQ ID NO: 111) | |
| 83 | CACTGAGTTTCGTCACCAGTACAAACTACAACGCCTGTAGCATTCCA | 60 |
| | CAGACAGCCCTCA (SEQ ID NO: 112) | |
| 84 | TAGTTAGCGTAACGATCTAAAGTTTTGTCGTCTTTCCAGACGTTAGT | 60 |
| | AAATGAATTTTCT (SEQ ID NO: 113) | |
| 85 | GTATGGGATTTTGCTAAACAACTTTCAACAGTTTCAGCGGAGTGAGA | 60 |
| | ATAGAAAGGAACA (SEQ ID NO: 114) | |
| 86 | ACTAAAGGAATTGCGAATAATAATTTTTTCACGTTGAAAATCTCCAAA | 60 |
| | AAAAAGGCTCCA (SEQ ID NO: 115) | |
| 87 | AAAGGAGCCTTTAATTGTATCGGTTTATCAGCTTGCTTTCGAGGTGA | 60 |
| | ATTTCTTAAACAG (SEQ ID NO: 116) | |
| 88 | CTTGATACCGATAGTTGCGCCGACAATGACAACAACCATCGCCCAC | 60 |
| | GCATAACCGATATA (SEQ ID NO: 117) | |
| 89 | TTCGGTCGCTGAGGCTTGCAGGGAGTTAAAGGCCGCTTTTGCGGG | 60 |
| | ATCGTCACCCTCAGC (SEQ ID NO: 118) | |
| 90 | CTTTTTCATGAGGAAGTTTCCATTAAACGGGTAAAATACGTAATGCC | 60 |
| | ACTACGAAGGCAC (SEQ ID NO: 119) | |
| 91 | CAACCTAAAACGAAAGAGGCAAAAGAATACACTAAAACACTCATCTT | 60 |
| | TGACCCCCAGCGA (SEQ ID NO: 120) | |
| 92 | TTATACCAAGCGCGAAACAAAGTACAACGGAGATTTGTATCATCGCC | 60 |
| | TGATAAATTGTGT (SEQ ID NO: 121) | |
| 93 | CGAAATCCGCGACCTGCTCCATGTTACTTAGCCGGAACGAGGCGCA | 60 |
| | GACGGTCAATCATA (SEQ ID NO: 122) | |
| 94 | AGGGAACCGAACTGACCAACTTTGAAAGAGGACAGATGAACGGTGT | 60 |
| | ACAGACCAGGCGCA (SEQ ID NO: 123) | |
| 95 | TAGGCTGGCTGACCTTCATCAAGAGTAATCTTGACAAGAACCGGAT | 60 |
| | ATTCATTACCCAAA (SEQ ID NO: 124) | |
| 96 | TCAACGTAACAAAGCTGCTCATTCAGTGAATAAGGCTTGCCCTGAC | 60 |
| | GAGAAACACCAGAA (SEQ ID NO: 125) | |
| 97 | CGAGTAGTAAATTGGGCTTGAGATGGTTTAATTTCAACTTTAATCATT | 60 |
| | GTGAATTACCTT (SEQ ID NO: 126) | |
| 98 | ATGCGATTTTAAGAACTGGCTCATTATACCAGTCAGGACGTTGGGAA | 60 |
| | GAAAAATCTACGT (SEQ ID NO: 127) | |
| 99 | TAATAAAACGAACTAACGGAACAACATTATTACAGGTAGAAAGATTC | 60 |
| | ATCAGTTGAGATT (SEQ ID NO: 128) | |
| 100 | TAAGAGCAACACTATCATAACCCTCGTTTACCAGACGACGATAAAAA | 60 |
| | CCAAAATAGCGAG (SEQ ID NO: 129) | |
| 101 | AGGCTTTTGCAAAAGAAGTTTTGCCAGAGGGGGTAATAGTAAAATGT | 60 |
| | TTAGACTGGATAG (SEQ ID NO: 130) | |
| 102 | CGTCCAATACTGCGGAATCGTCATAAATATTCATTGAATCCCCCTCA | 60 |
| | AATGCTTTAAACA (SEQ ID NO: 131) | |
| 103 | GTTCAGAAAACGAGAATGACCATAAATCAAAAATCAGGTCTTTACCC | 60 |
| | TGACTATTATAGT (SEQ ID NO: 132) | |
| 104 | CAGAAGCAAAGCGGATTGCATCAAAAAGATTAAGAGGAAGCCCGAA | 60 |
| | AGACTTCAAATATC (SEQ ID NO: 133) | |
| 105 | GCGTTTTAATTCGAGCTTCAAAGCGAACCAGACCGGAAGCAAACTC | 60 |
| | CAACAGGTCAGGAT (SEQ ID NO: 134) | |
| 106 | TAGAGAGTACCTTTAATTGCTCCTTTTGATAAGAGGTCATTTTTGCG | 60 |
| | GATGGCTTAGAGC (SEQ ID NO: 135) | |
| 107 | TTAATTGCTGAATATAATGCTGTAGCTCAACATGTTTTAAATATGCAA | 60 |
| | CTAAAGTACGGT (SEQ ID NO: 136) | |
| 108 | GTCTGGAAGTTTCATTCCATATAACAGTTGATTCCCAATTCTGCGAA | 60 |
| | CGAGTAGATTTAG (SEQ ID NO: 137) | |
| 109 | TTTGACCATTAGATACATTTCGCAAATGGTCAATAACCTGTTTAGCTA | 49 |
| | T (SEQ ID NO: 138) | |

It is contemplated that certain alterations can be made to the above-referenced sequences, thus sequences suitable for use herein may, in embodiments, included nucleic acid sequences including or consisting of amino acid sequences having at least 80%, 90%, 95%, 97%, and 99% sequence identity to the sequences listed above in Table 5.

TABLE 6
Variable oligos

| Name | Sequence (5′-3′) | Length |
|---|---|---|
| Var 1 | AACATCCAATAAATCATACAGGCAAGGCAAAGAATTAGCAAAATTA | 60 |
| | AGCAATAAAGCCTC (SEQ ID NO: 139) | |
| Var 2 | GTGAGCGAGTAACAACCCGTCGGATTCTCCGTGGGAACAAACGG | 60 |
| | CGGATTGACCGTAATG (SEQ ID NO: 140) | |
| Var 3 | TTCTTTTCACCAGTGAGACGGGCAACAGCTGATTGCCCTTCACCG | 60 |
| | CCTGGCCCTGAGAGA (SEQ ID NO: 141) | |
| Var 4 | TCTGTCCATCACGCAAATTAACCGTTGTAGCAATACTTCTTTGATT | 60 |
| | AGTAATAACATCAC (SEQ ID NO: 142) | |
| Var 5 | ATTCGACAACTCGTATTAAATCCTTTGCCCGAACGTTATTAATTT | 60 |
| | TAAAAGTTTGAGTAA (SEQ ID NO: 143) | |
| Var 6 | TGGGTTATATAACTATATGTAAATGCTGATGCAAATCCAATCGCA | 60 |
| | AGACAAAGAACGCGA (SEQ ID NO: 144) | |
| Var 7 | GTTTTAGCGAACCTCCCGACTTGCGGGAGGTTTTGAAGCCTTAA | 60 |
| | ATCAAGATTAGTTGCT (SEQ ID NO: 145) | |
| Var 8 | TCAACCGATTGAGGGAGGGAAGGTAAATATTGACGGAAATTATT | 60 |
| | CATTAAAGGTGAATTA (SEQ ID NO: 146) | |
| Var 9 | GTATTAAGAGGCTGAGACTCCTCAAGAGAAGGATTAGGATTAGCG | 60 |
| | GGGTTTTGCTCAGTA (SEQ ID NO: 147) | |
| Var | AGCGAAAGACAGCATCGGAACGAGGGTAGCAACGGCTACAGAG | 60 |
| 10 | GCTTTGAGGACTAAAGA (SEQ ID NO: 148) | |
| Var | TAGGAATACCACATTCAACTAATGCAGATACATAACGCCAAAAGG | 60 |
| 11 | AATTACGAGGCATAG (SEQ ID NO: 149) | |
| Var | ATTTTCATTTGGGGCGCGAGCTGAAAAGGTGGCATCAATTCTACT | 60 |
| 12 | AATAGTAGTAGCATT (SEQ ID NO: 150) | |

It is contemplated that certain alterations can be made to the above-referenced sequences, thus sequences suitable for use herein may, in embodiments, included nucleic acid sequences including or consisting of amino acid sequences having at least 80%, 90%, 95%, 97%, and 99% sequence identity to the sequences listed above in Table 6.

TABLE 7
Filler oligos

| Name | Sequence (5′-3′) | Length |
|---|---|---|
| Var 4 filler | TCTGTCCATCACGCAAATTA (SEQ ID NO: 151) | 20 |
| Var 5 filler | AATTTTAAAAGTTTGAGTAA (SEQ ID NO: 152) | 20 |
| Var 6 filler | TCGCAAGACAAAGAACGCGA (SEQ ID NO: 153) | 20 |
| Var 7 filler | TCGCAAGACAAAGAACGCGA (SEQ ID NO: 154) | 20 |
| Var 8 filler | TATTCATTAAAGGTGAATTA (SEQ ID NO: 155) | 20 |
| Var 9 filler | TAGCGGGGTTTTGCTCAGTA (SEQ ID NO: 156) | 20 |

It is contemplated that certain alterations can be made to the above-referenced sequences, thus sequences suitable for use herein may, in embodiments, included nucleic acid sequences including or consisting of amino acid sequences having at least 80%, 90%, 95%, 97%, and 99% sequence identity to the sequences listed above in Table 7.

TABLE 8
Other oligos

| | | |
|---|---|---|
| Blocking | TCTCATGGCCCTTC (SEQ ID NO: 157) | 14 |
| BtsCI cut | CTACTAATAGTAGTAGCATTAACATCCAATAAATCA | 40 |
| site oligo | TACA (SEQ ID NO: 158) | |

It is contemplated that certain alterations can be made to the above-referenced sequences, thus sequences suitable for use herein may, in embodiments, included nucleic acid sequences including or consisting of amino acid sequences having at least 80%, 90%, 95%, 97%, and 99% sequence identity to the sequences listed above in Table 8.

### Example

### Materials and Methods

### DNA Nanoswitches

DNA nanoswitches were designed and fabricated according to the protocol presented in Chandrasekaran, A. R. et al. Cellular microRNA detection with miRacles: microRNA-activated conditional looping of engineered switches. Science Advances 5, eaau9443 (2019) (herein entirely incorporated by reference). (See also Lifeng Zhou, Cassandra Cavaliere, Andrew Hayden, Paromita Dey, Song Mao, Arun Richard Chandrasekaran, Jia Sheng, Bijan K. Dey, Ken Halvorsen, Single Species RNA Purification with DNA nanoswitches, bioRxiv 2020.07.07.191338; doi: https://doi.org/10.1101/2020.07.07.191338 (herein entirely incorporated by reference).

Briefly, circular M13 ssDNA (New England Biolabs) was linearized by enzyme BtsCl (New England Biolabs). The backbone and detection oligos (IDT DNA) were mixed with the linearized M13 with about 10× excess (all oligos used in this research are presented in Tables 1 to 5 above). All nanoswitches were self-assembled in a thermal cycler with a thermal annealing protocol (90 to 25° C., 1° C./min). After fabrication, DNA nanoswitches were purified by either HPLC or by PEG precipitation. See e.g, Chandrasekaran, A. R. et al. Cellular microRNA detection with miRacles: microRNA-activated conditional looping of engineered switches. Science Advances 5, eaau9443 (2019). Purified nanoswitches were resuspended in 1×PBS and their concentrations were measured using a Nanodrop 2000.

### RNA Samples

The 401 nt mRNA fragment (Table 1) was produced by in vitro transcription (NEB, HiScribe™ T7 High Yield RNA Synthesis Kit) from a DNA template (Table 1 and 2), which was a gift from Prof. Prashanth Rangan. The microRNA target (miR-206) was commercially synthesized (IDT DNA). Total RNA from HeLa cells was purchased from BioChain Institute Inc.

RNA strand containing the m^{4,4}C modification was synthesized at 1.0 mmol scale by solid phase synthesis using an Oligo-800 synthesizer. After synthesis, the oligos were cleaved from the solid support and fully deprotected with AMA (ammonium hydroxide:methylamine solution=1:1) at 65° C. for 45 min. The amines were removed by Speed-Vac concentrator followed by Triethylamine trihydrofluoride (Et3N.3HF) treatment for 2.5 h at 65° C. to remove the TBDMS protecting groups. Cooled down to room temperature the RNA was precipitated by adding 0.025 mL of 3 M sodium acetate and 1 mL of ethanol. The solution was cooled to −80° C. for 1 h before the RNA was recovered by centrifugation and finally dried under vacuum. The RNA strands were then purified by 15% denaturing polyacrylamide gel electrophoresis (PAGE) and were desalted, concentrated and lyophilized before redissolving in RNase free water

### Nanoswitch Detection Assays

Unless otherwise noted, all nanoswitch detection and assays were performed by incubating the nanoswitches with target RNA in 1×PBS and 10 mM MgCl₂ in a thermal annealing ramp (40 to 25° C., 0.1° C./min) for about 12 hours. Nanoswitch concentrations varied from ˜0.1-1 nM as noted in figure captions, with typically high nanoswitch concentrations for high capture in the purification process and low concentrations for validation assays. Validation assays also included 200 nM of “blocking oligos” to minimize RNA sticking to the tubes. GelRed DNA stain and a Ficoll-based loading dye were added to the final samples at a 3.3× and 1× final concentrations, respectively. Gel electrophoresis was performed using 0.8% agarose gels in 0.5×TBE buffer. Gels were run at 4° C. at 60-75V for 45 minutes to 2 hours depending on the assay.

### RNA Purification

After performing the nanoswitch detection assay and running gel electrophoresis, the looped nanoswitch gel bands were excised on the image platform of a Gel Doc XR+ System (Bio-Rad) by using disposable plastic gel cutting tool and razor (Sigma-Aldrich). All gel bands were diced into small pieces before transfer into 1.5 ml tubes. Then, the gel pieces were submerged in 1×DNase I buffer (NEB) and 4 U DNase I (NEB) was used for each 200 μl to digest the DNA nanoswitches at 37° C. for 1 hour. Following DNase I digestion, Zymoclean Gel RNA recovery kit (Zymo research) was used to recover and clean the target RNA. The manufacturer's instructions were followed, except for doing two sequential elutions for the last step in 10 μl nuclease-free water each. Totally, for each column, we obtained 20 μl RNA sample. An optional 15-minute heating step at 90° C. was used to destroy any residual DNase I before downstream re-detection by the DNA nanoswitches.

### Modification Analysis by LC-MS

Measurement of the level of m^{4,4}C was performed by ultra-performance liquid chromatography coupled with tandem mass spectrometry (UHPLC-MS/MS) using a method similar to that previously described in Tardu, M., Jones, J. D., Kennedy, R. T., Lin, Q. & Koutmou, K. S. Identification and Quantification of Modified Nucleosides in Saccharomyces cerevisiae mRNAs. ACS Chem. Biol. 14, 1403-1409 (2019). After the purification of the RNA with m^{4,4}C modification, the inventors first dried the sample in a universal vacuum system (Savant UVS 400) and then resuspended in RNase-free water and digested the RNA into nucleotides by Nucleoside Digestion Mix (NEB, M0649S) in 15 μl volume and used 10 μl for the test. The UHPLC-MS/MS analysis was accomplished on a Waters XEVO TQ-S™ (Waters Corporation, USA) triple quadruple tandem mass spectrometer equipped with an electrospray source (ESI) maintained at 150° C. and a capillary voltage of 1 kV. Nitrogen was used as the nebulizer gas which was maintained at 7 bars pressure, flow rate of 1000 l/h and at a temperature of 500° C. UHPLC-MS/MS analysis was performed in ESI positive-ion mode using multiple-reaction monitoring (MRM) from ion transitions previously determined for m^{4,4}C (m/z 272>140). A Waters ACQUITY UPLC™ HSS T3 guard column (2.1×5 mm, 1.8 μm) attached to a HSS T3 column (2.1×50 mm, 1.7 μm) was used for the separation. Mobile phases included RNAse-free water (18 MΩcm⁻¹) containing 0.01% formic acid (Buffer A) and 50% acetonitrile (v/v) in Buffer A (Buffer B). The digested nucleotides were eluted at a flow rate of 0.4 ml/min with a gradient as follows: 0-2 min, 0-10% B; 2-3 min, 10-15% B; 3-4 min, 15-100% B; 4-4.5 min, 100% B. The total run time was 7 min. The column oven temperature was kept at 35° C. and sample injection volume was 10 μl. Three injections were performed for each sample. Data acquisition and analysis were performed with MassLynx V4.1 and TargetLynx. Calibration curves were plotted using linear regression with a weight factor of 1/concentration (1/x).

qRT-PCR Assays

cDNA synthesis of purified mRNA fragment was carried out using the iScript cDNA Synthesis Kit (Bio-Rad) as instructed. The various amount of known mRNA fragments (0.0625 nM, 0.125 nM, 0.25 nM, 0.5 nM, and 1 nM) were similarly converted into cDNAs and used to generate a standard curve. Then, qRT-PCR was carried out using SYBR green PCR master mix (Bio-Rad) in a Bio-Rad Realtime Thermal Cycler using purified fragment specific primers (Forward-TGTTTGCTTTCGTGAAAACTCGCAT) (SEQ ID NO: 159); Reverse-ACATTAGGTGCAATACCGAAGGCA (SEQ ID NO: 160). The concentration of unknown purified fragments was derived from the known amount of fragments used to generate standard curves.

cDNA synthesis of purified miR-206 was carried out using miRCURY LNA RT Kit (Qiagen) as described. Briefly, every 10 μl RT reaction contained 2 μl of 5× reaction buffer, 4.5 μl of RNase free water, 1 μl of 10×RT enzyme mix, 0.5 μl of RNA spike in SP6 and 2 μl of template microRNA. The reactions were thoroughly mixed in 0.2 ml tubes and subjected to cycling: 60 min at 42° C., 5 min at 95° C. Similarly known amount of synthetic miR-206 (0.0625 nM, 0.125 nM, 0.25 nM, 0.5 nM, and 1 nM) were converted into cDNAs and used to generate a standard curve for calculating unknown miR-206 concentration. qRT-PCR for microRNA was done following the instructions of miRCURY SYBR Green PCR Kit (Qiagen). Briefly, for every 10 μl reaction, 5 μl of 2×miRCURY SYBR Green Master Mix, 2 μl of Nuclease Free Water, 1 μl of miR-206 PCR Primer Mix, and 2 μl of cDNA Template were used. The reactions were mixed well and dispensed into PCR plate (10 μl/well), which was then placed in a Bio-Rad Realtime Thermal Cycler using the cycling protocol: after 2 minutes at 95° C., 40 repetitions of 10 seconds at 95° C. and 60 seconds at 56° C. The melt curve analysis was done at 60-95° C. The concentration of unknown purified miR-206 was derived from the known amount of miR-206 used to generate standard curves.

### Single Species RNA Purification Based on DNA Nanoswitches

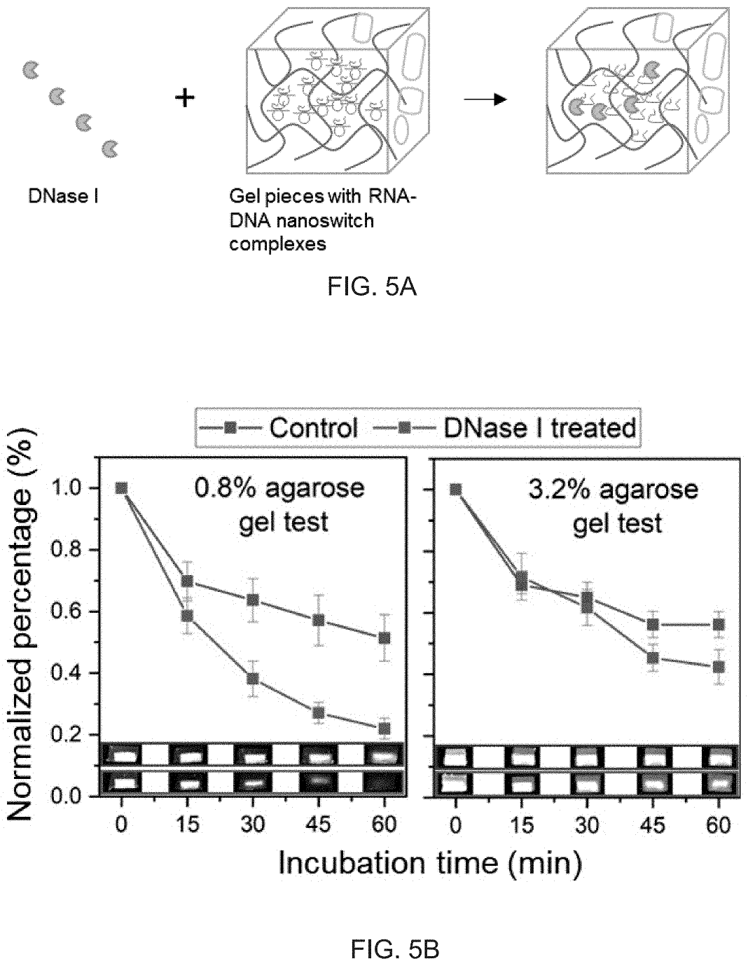

A detect-and-purify strategy of the present disclosure for single species RNA purification was implemented overcoming many drawbacks of current approaches is shown in FIG. 3. Here, the inventors used DNA nanoswitches of the present disclosure configured to change conformation upon binding to a targeted sequence such as an RNA sequence. In embodiments, the nanoswitch of the present disclosure is a linear double stranded DNA (dsDNA) with two ssDNA capture probes (oligo sequences presented in Tables 1 to 5) that cause it to reconfigure to a looped dsDNA upon binding an RNA target (FIG. 3B)⁸. In embodiments, the strategy includes three process sequences: 1) isolation of an RNA-looped nanoswitches by gel electrophoresis and gel excision; 2) digestion of DNA nanoswitches and gel pieces; and 3) removal of digested byproducts. First, the DNA nanoswitches capture target RNA molecules and become looped. Looped nanoswitches are separated and imaged using gel electrophoresis and isolated using gel excision. The nanoswitches are then digested using DNase I, which we found to work even on intact gel pieces (FIG. 5). The last step removes byproducts using a commercially available kit to dissolve the agarose gel pieces and then to purify the RNA from enzymes and nanoswitch fragments (FIG. 3C).

As shown in FIG. 3A, a plurality of RNA molecules 305 such as a mixture 307 may be present in an initial sample. It is very challenging to isolate and purify any single species from a biological mixture including a plurality of RNA molecules, or other biological products. Referring to FIG. 3B, a DNA nanoswitch 309 of the present disclosure is shown converting from a liner form 309 to a looped form 311 in the presence of a target RNA molecule 313. Referring to FIG. 3C, a purification process sequence of the present disclosure may include, in embodiments, detecting (as shown in step 1 (315) and step 2 (317)) and purifying (steps 3 (319), step 4 (321) and step 5 (323)) RNA.

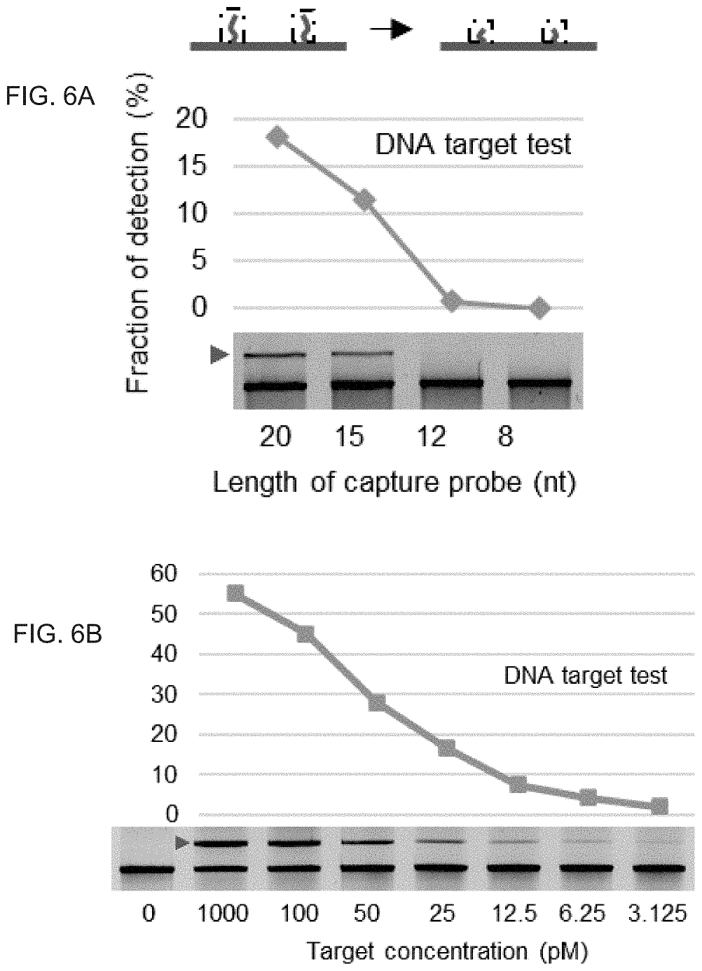

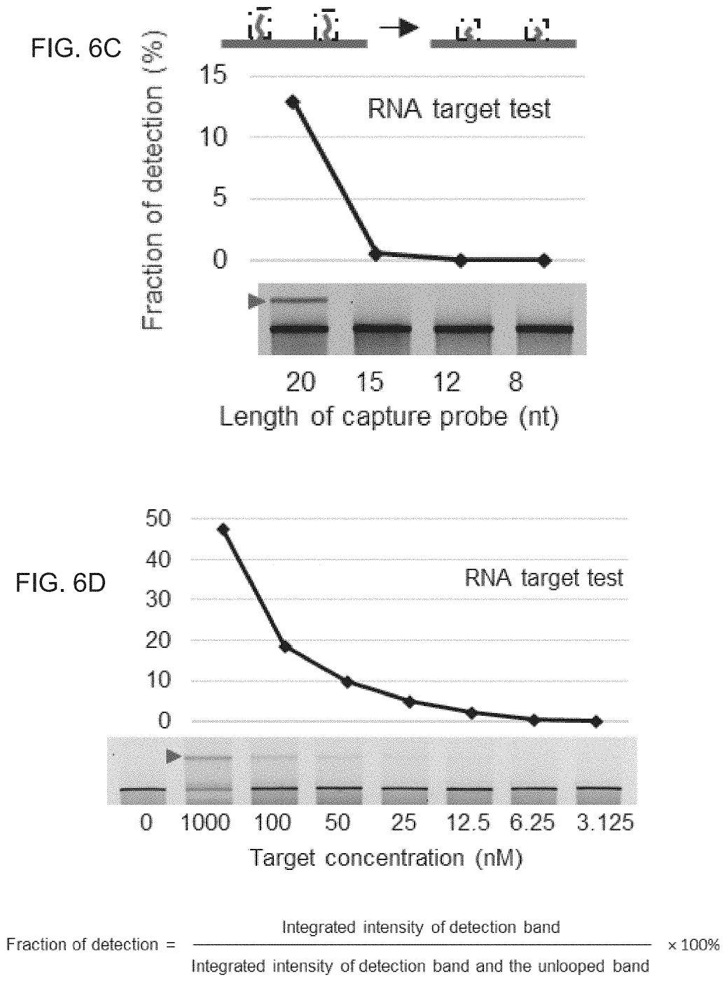

To demonstrate the purification, two target RNA molecules were provided with different lengths: a 401 nucleotide (nt) transcribed RNA fragment from the 3′ untranslated region of a Drosophila mRNA (See e.g., Flora, P. et al. Sequential Regulation of Maternal mRNAs through a Conserved cis-Acting Element in Their 3′ UTRs. Cell Reports 25, 3828-3843.e9 (2018) and a synthetic 22 nt microRNA-206 (miR-206). A target region (40 nt) on the mRNA fragment (Table 1) was chosen. Four versions of nanoswitches with different capture probe lengths were designed and it was found that the nanoswitch with 20 nt was the most efficient, with a detection limit of ˜12.5 pM for the mRNA fragment (FIG. 6). For miR-206, it was demonstrated sub-picomolar detection ability and adopted the same nanoswitch design here for the detection and purification. See e.g., Chandrasekaran, A. R. et al. Cellular microRNA detection with miRacles: microRNA-activated conditional looping of engineered switches. Science Advances 5, eaau9443 (2019).

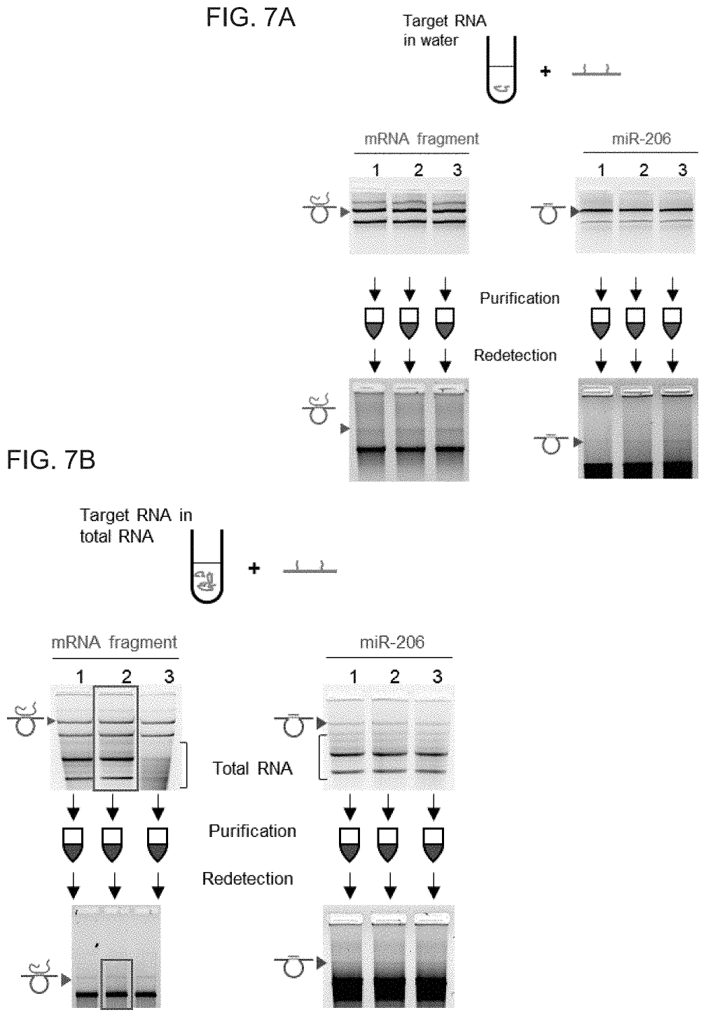

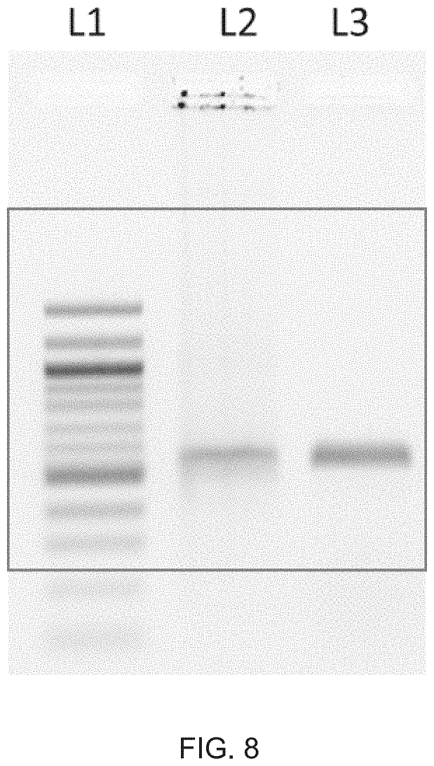

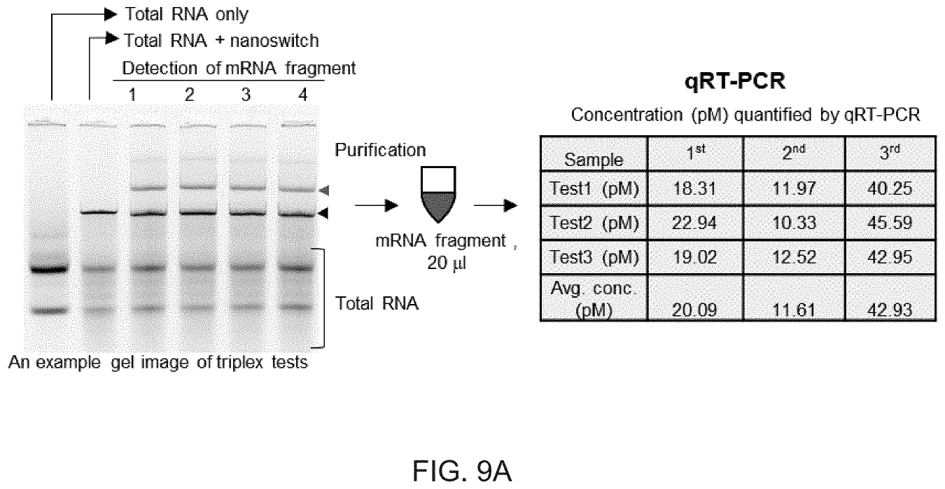

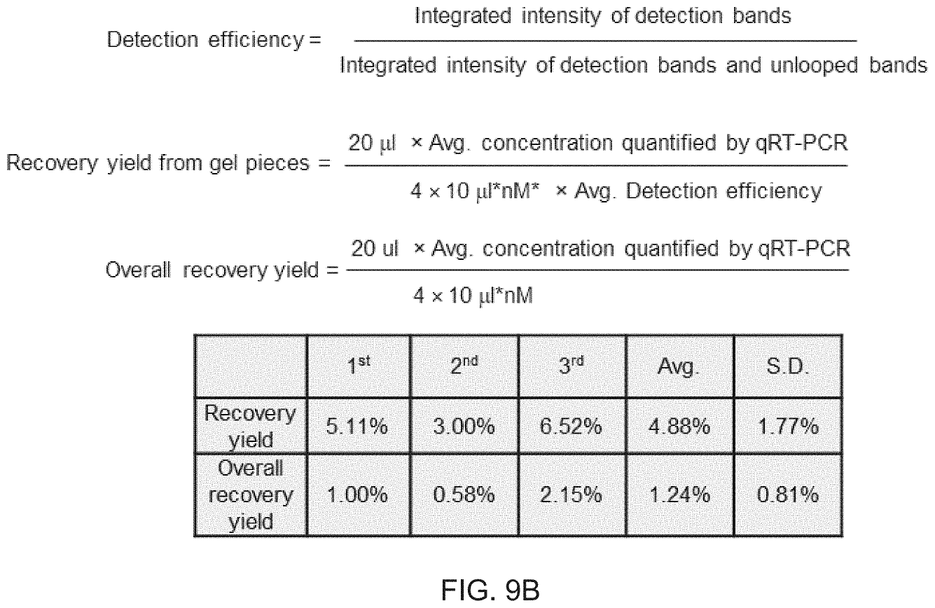

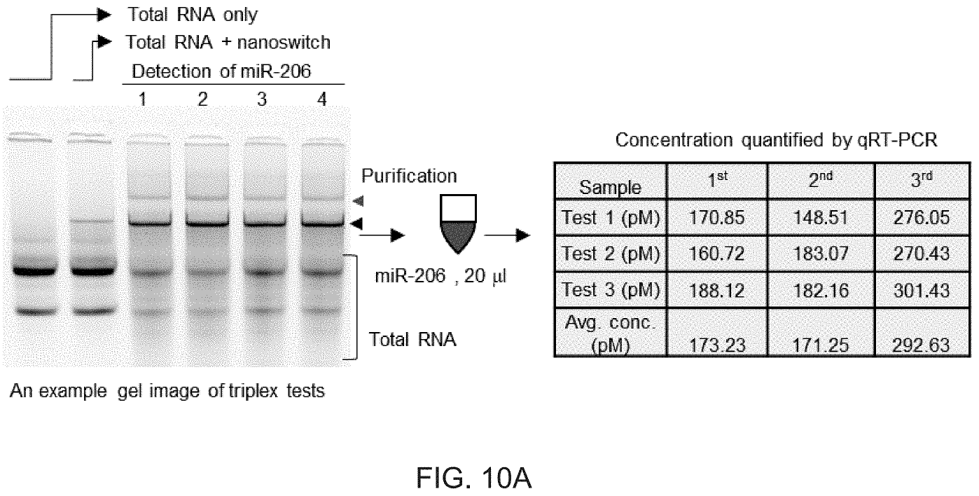

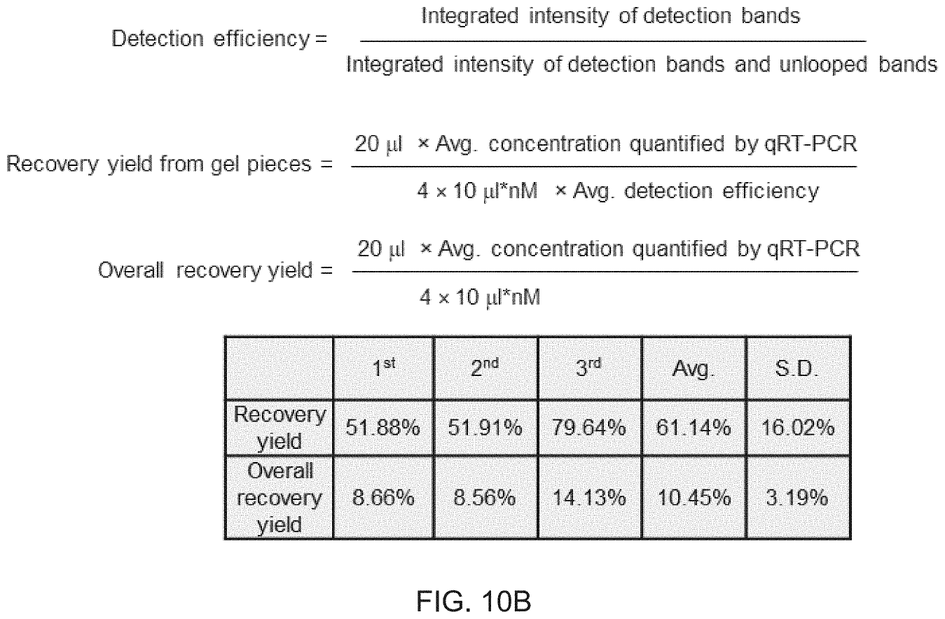

As proof-of-concept, a purification procedure was performed for the mRNA fragment and miR-206 in water as well as spiked into total RNA to mimic a biological context and targets in both cases were recovered (FIG. 3D and FIG. 7). Successful purification was demonstrated of the correct sequence by re-detecting the purified product with the nanoswitches (FIG. 3D). To further validate the purification and quantify the yield, qRT-PCR was performed on the purified products. Both products were successfully amplified, and the mRNA fragment was consistent with the full-length DNA template used for transcription (FIG. 3D and FIG. 8). The final amounts determined by qRT-PCR were compared to the captured RNA and the starting RNA to determine the recovery yield and overall yield, respectively. Overall yield of mRNA fragment and miR-206 was found to be 1.2% and 10.5% (FIG. 3E, FIG. 5 and FIG. 6).

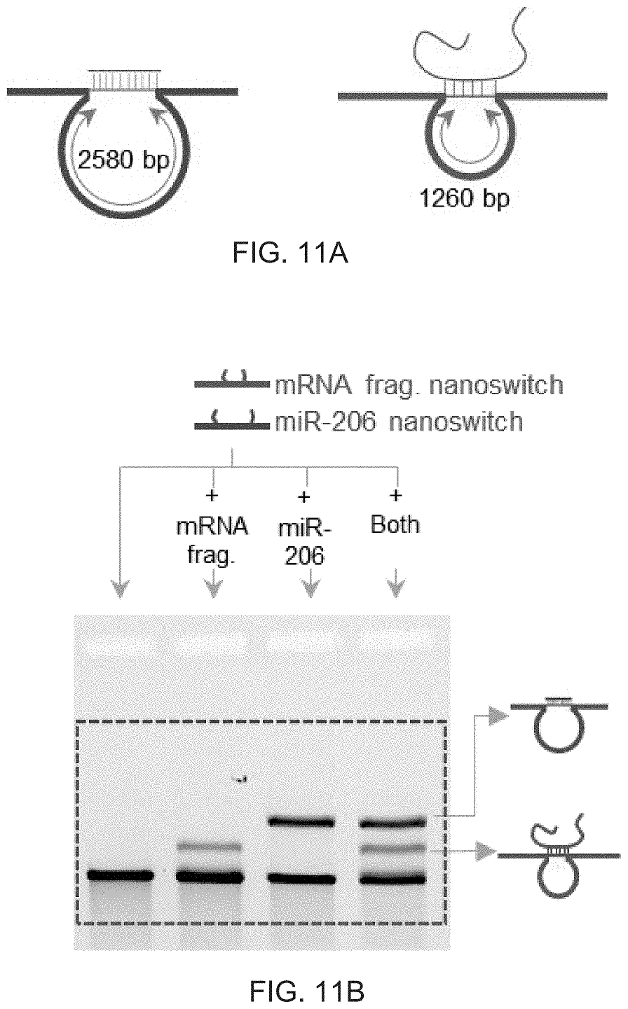

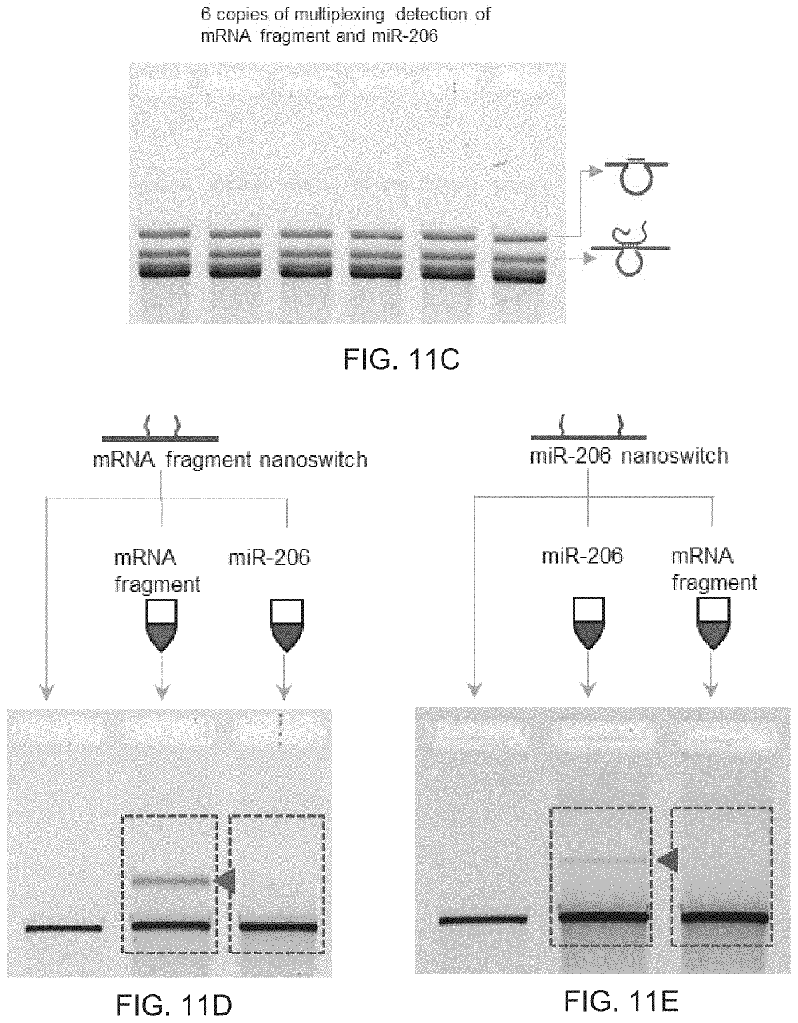

A unique feature of the DNA nanoswitches is that they can be programmed to enable multiplexing (See e.g., Chandrasekaran, A. R. et al. Cellular microRNA detection with miRacles: microRNA-activated conditional looping of engineered switches. Science Advances 5, eaau9443 (2019), which we used here for simultaneous purification of multiple RNA molecules from a single reaction. This cannot be easily accomplished by other methods such as bead-based purification. To achieve this, the capture probes were positioned for the mRNA fragment nanoswitch to form a smaller loop (with faster migration) than the looped nanoswitch of miR-206 (FIG. 7). With this configuration, detection and individual purification of the mRNA fragment and miR-206 at the same time was shown (FIG. 3F). Redetection of the purified products with nanoswitches demonstrated the specificity of the purification method. Each nanoswitch only detected the correct purified target (mRNA or miR-206), indicating successful isolation of target RNA without notable cross-contamination (FIG. 11). Based on previous multiplexed detection, this should be scalable to at least 5 different RNA molecules in a single reaction (See e.g., Chandrasekaran, A. R., Levchenko, O., Patel, D. S., MacIsaac, M. & Halvorsen, K. Addressable configurations of DNA nanostructures for rewritable memory. Nucleic Acids Res 45, 11459-11465 (2017).

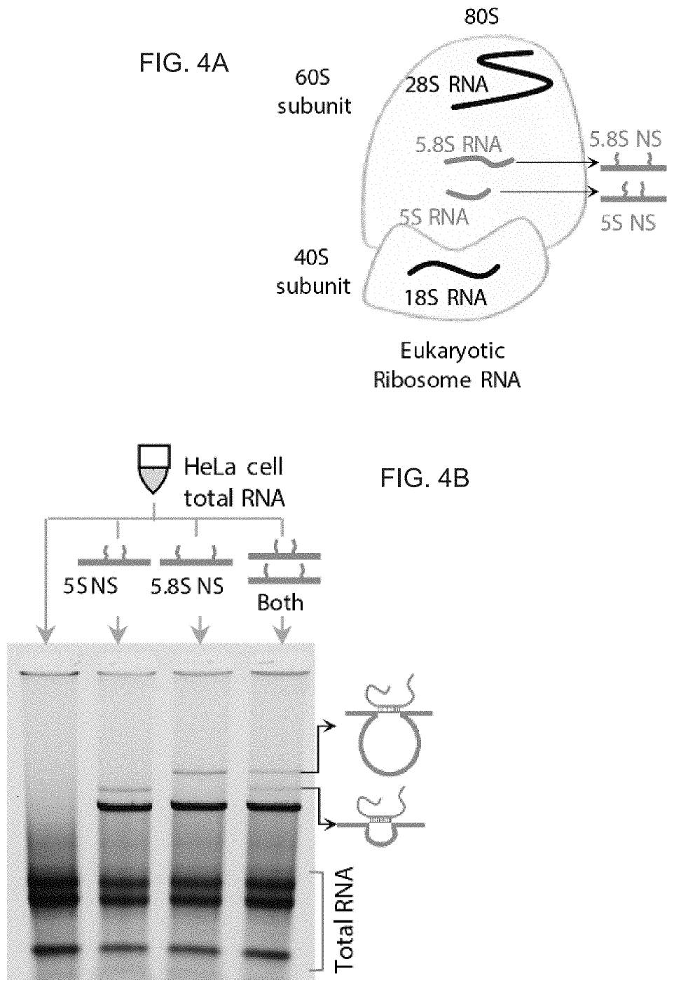

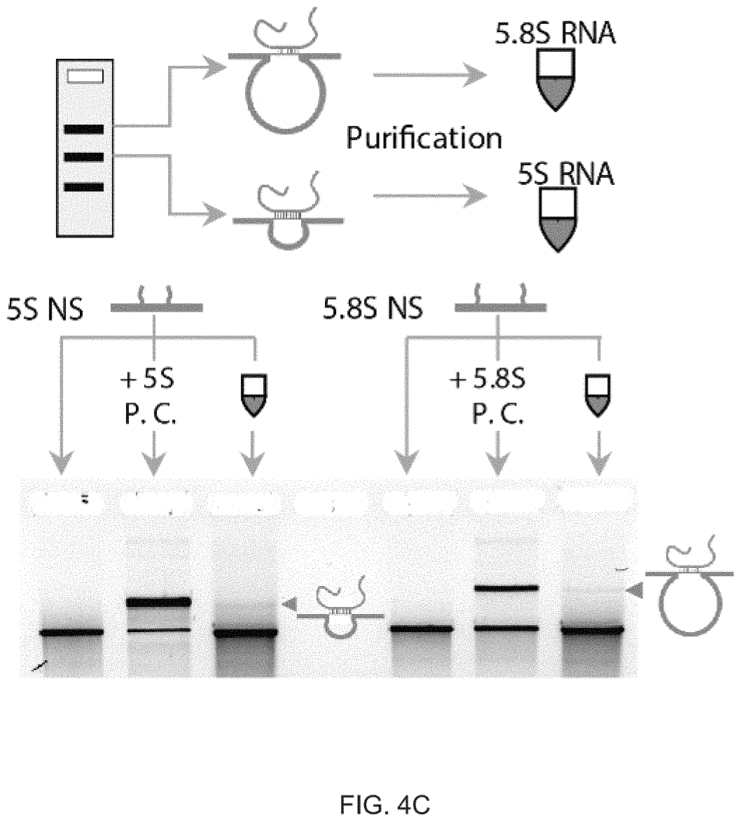

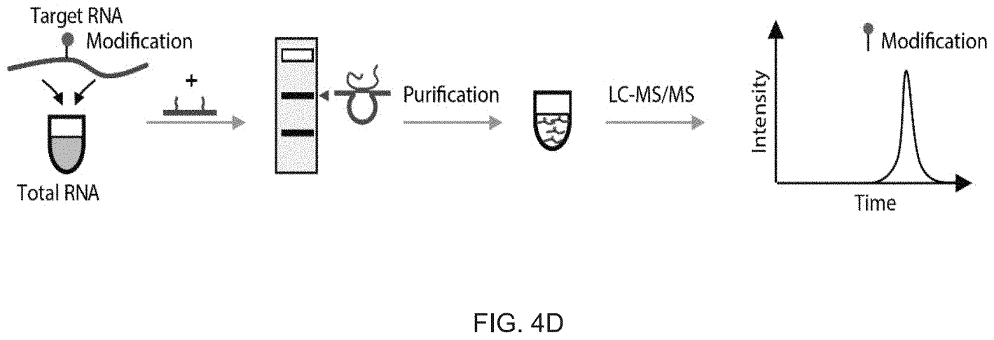

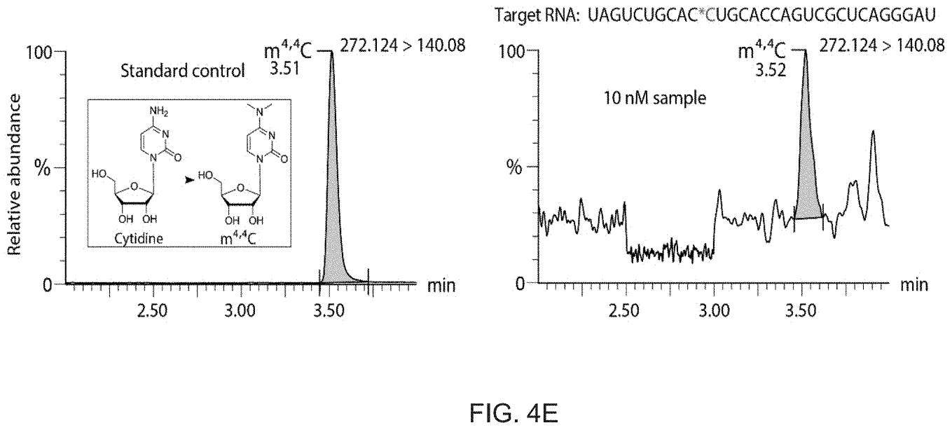

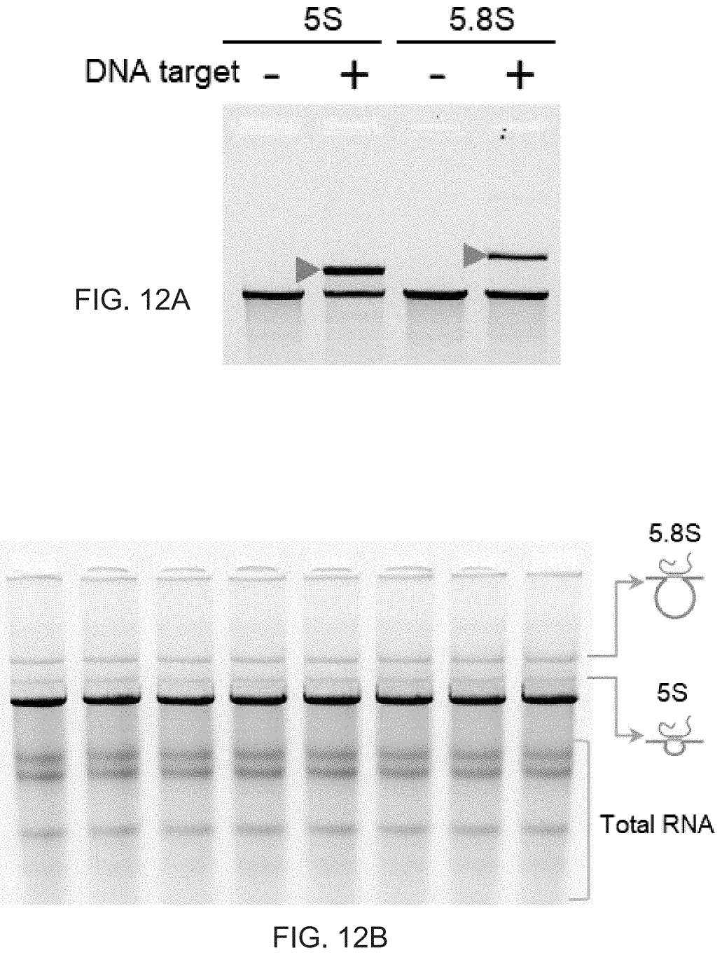

To demonstrate purification of RNAs from real biological samples, multiplexed nanoswitches targeting the 5.8S and 5S ribosomal RNAs (rRNAs) from HeLa cell total RNA was developed (FIG. 4A). The 5.8S and 5S subunits (156 and 121 nt respectively) are critical for protein translation (See e.g., Gillespie, J. J., Johnston, J. S., Cannone, J. J. & Gutell, R. R. Characteristics of the nuclear (18S, 5.8S, 28S and 5S) and mitochondrial (12S and 16S) rRNA genes of Apis mellifera (Insecta: Hymenoptera): structure, organization, and retrotransposable elements. Insect Mol Biol 15, 657-686 (2006)) and have been shown to contain chemical modifications including pseudouridine. (See e.g., Decatur, W. A. & Schnare, M. N. Different Mechanisms for Pseudouridine Formation in Yeast 5S and 5.8S rRNAs. Mol Cell Biol 28, 3089-3100 (2008) and Taoka, M. et al. Landscape of the complete RNA chemical modifications in the human 80S ribosome. Nucleic Acids Res 46, 9289-9298 (2018)). Target regions were chosen for each based on secondary structure analysis and designed nanoswitches to form a large loop for 5.8S rRNA and a small loop for 5S rRNA (FIG. 12). Clear multiplexed detection of the 5.8S and 5S rRNAs directly from 250 ng total RNA from HeLa cells was shown (FIG. 4B). Following the established workflow, one separately purified each rRNA species from 2 μg total RNA of HeLa cell (FIG. 12) and confirmed successful purification of the correct rRNA in each instance (FIG. 4C).

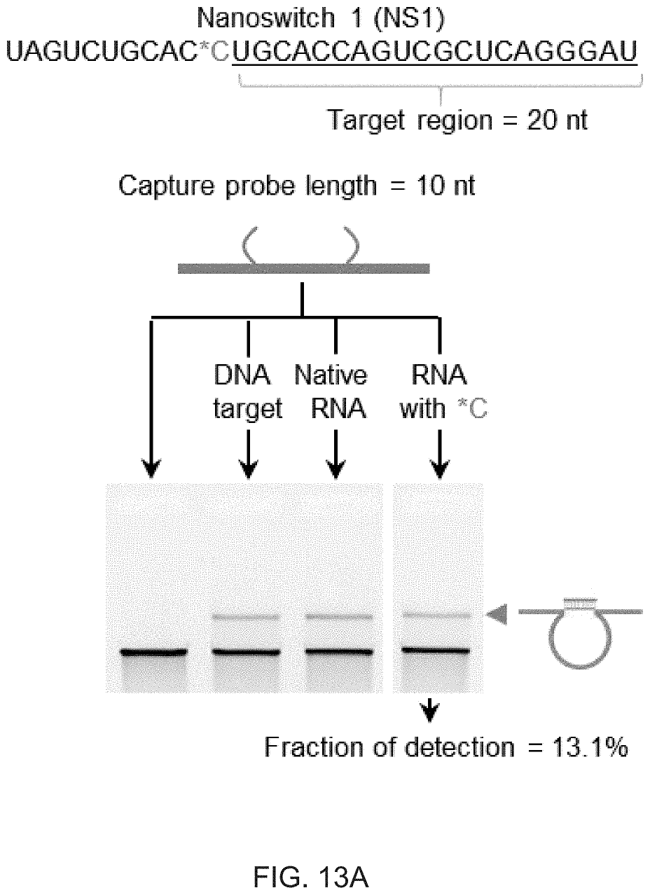

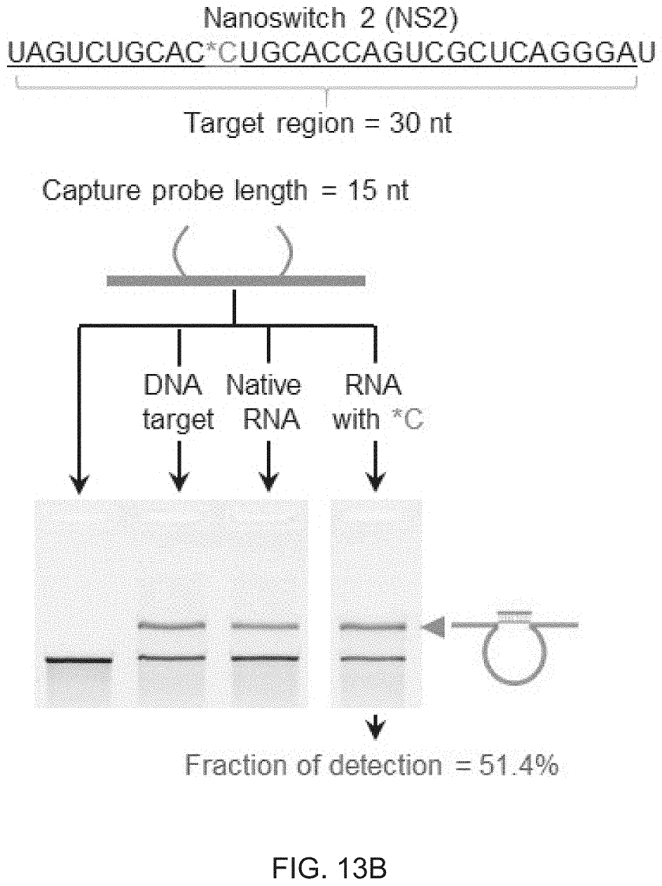

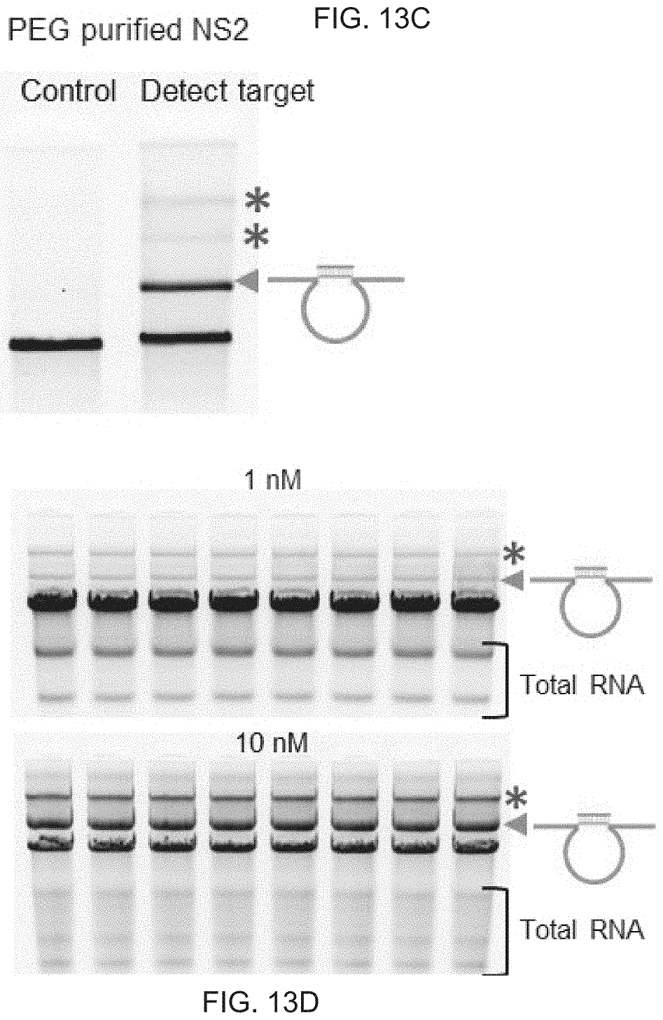

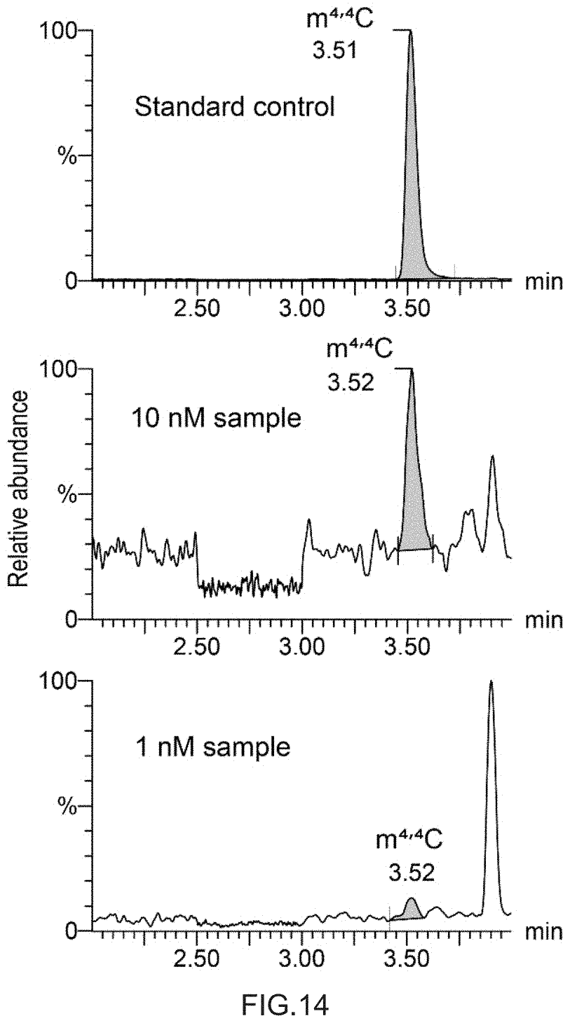

One compelling application of the approach is to study how the >100 natural RNA modifications can alter biological functions of particular RNAs. See e.g., Boccaletto, P. et al. MODOMICS: a database of RNA modification pathways. 2017 update. Nucleic Acids Res 46, D303-D307 (2018). For example, N6-methyladenosine (m⁶A) affects the stability of mRNA and protein translation (See e.g., Wang, X. et al. m⁶A-dependent regulation of messenger RNA stability. Nature 505, 117-120 (2014), rRNA modifications can influence translation efficiency See e.g, Sloan, K. E. et al. Tuning the ribosome: The influence of rRNA modification on eukaryotic ribosome biogenesis and function. RNA Biol 14, 1138-1152 (2016), and some RNA modifications appear in response to viral infection (See e.g., McIntyre, W. et al. Positive-sense RNA viruses reveal the complexity and dynamics of the cellular and viral epitranscriptomes during infection. Nucleic Acids Res 46, 5776-5791 (2018)). One of the gold standard methods for measuring RNA modifications is ultra high-performance liquid chromatography-tandem Mass Spectrometry (UHPLC-MS/MS) (See e.g., Basanta-Sanchez, M., Temple, S., Ansari, S. A., D'Amico, A. & Agris, P. F. Attomole quantification and global profile of RNA modifications: Epitranscriptome of human neural stem cells. Nucleic Acids Res 44, e26 (2016)), but this method typically uses digested RNA and loses sequence information. Used in conjunction with our purification method, RNA modifications could be measured on specific RNA sequences (FIG. 4D). To demonstrate this application, the m^{4,4}C (N⁴, N⁴-dimethylcytidine) modification was chosen (FIG. 4E), which has relevance in viral infection and to our knowledge can only be identified by mass spectrometry methods. One synthesized and a PAGE purified short (31 nt) RNA with a single m^{4,4}C modification, and screened two nanoswitch designs since the modification can influence base pairing (FIG. 13). To mimic biological samples, the modified RNA was spiked at different concentrations (10 nM and 1 nM) into 2 μg of total RNA from HeLa cells and performed the purification (FIG. 13). Detection of the modification from the purified samples at both concentrations was shown, demonstrating that our method can be applied to extract target RNA with chemical modifications with enough material for downstream UHPLC-MS/MS testing (FIG. 4E and FIG. 14).

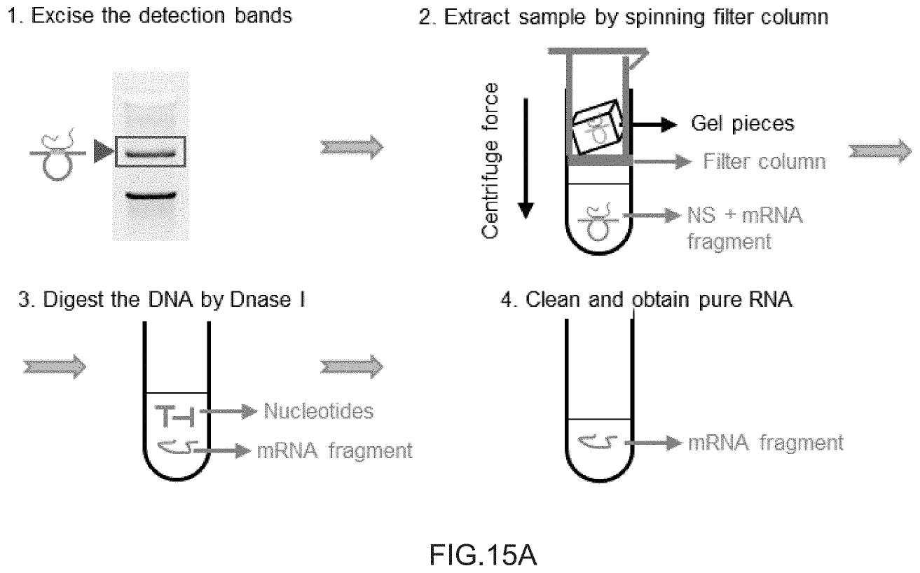

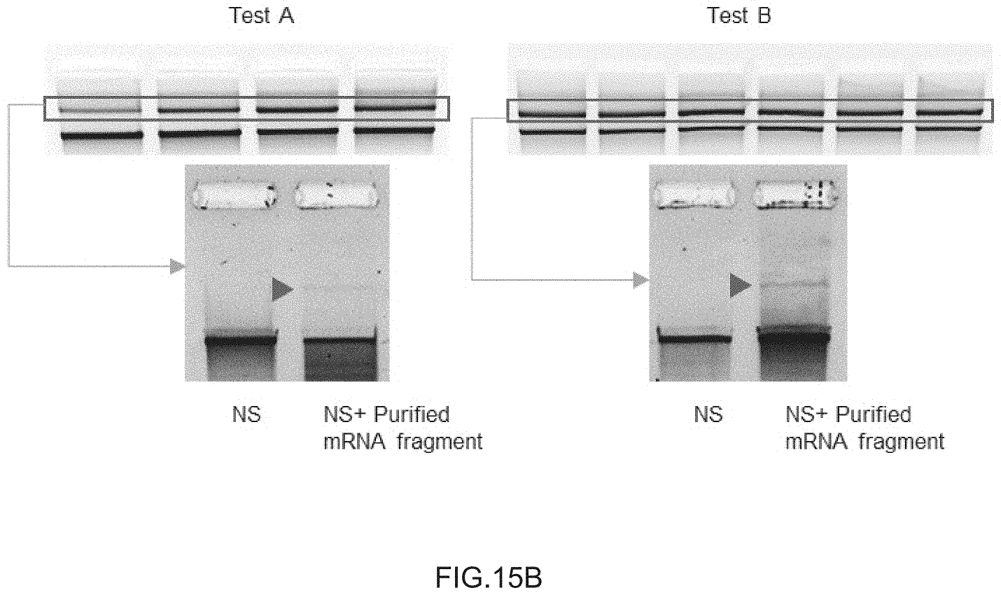

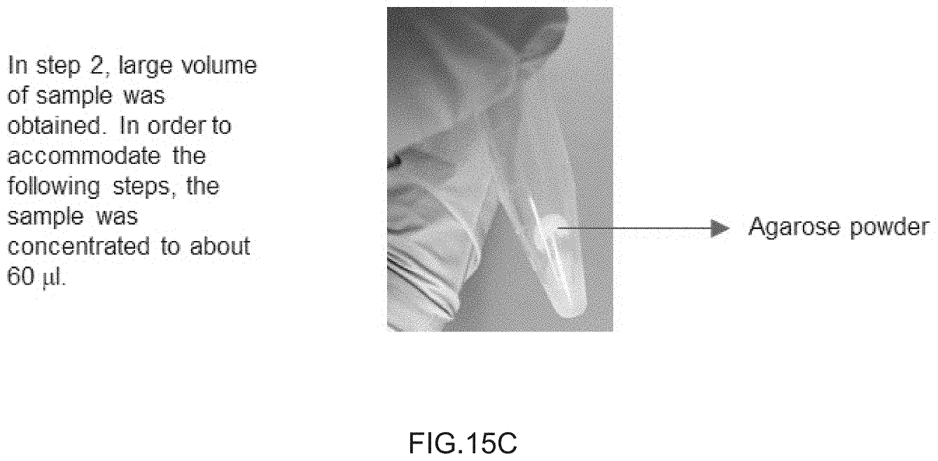

In embodiments, the methods of the present disclosure for single species RNA purification enables robust purification of diverse types of RNA from microgram scale samples in just a few hours. The capture probes of our nanoswitches can be readily programmed for multiple purification targets without significant effort. The approach of the present disclosure avoids the downsides of surface binding approaches present in bead-based methods, provides visual feedback of the process for troubleshooting, and can be performed at low cost on the benchtop (Table 6). It is envisioned that embodiments, are suitable for increase the overall yield and purification scale, expand multiplexing, and further reduce the cost and time for processing. Some other techniques could potentially speed or improve the collection of looped nanoswitches such as the BluePippin gel cassette (See e.g., Durin, G., Boles, C. & Ventura, P. Complementary DNA Shearing and Size-selection Tools for Mate-pair Library Construction. J Biomol Tech 23, S36-S37 (2012) or better spin columns (DNA gel extraction columns suffered from low yield—See FIG. 15).

Analysis of chemical modifications of RNA is a particularly attractive application. Mass spectrometry techniques can detect modifications at below fmol scale but tend to lose sequence information. By purifying single RNA species for MS (See e.g., Basanta-Sanchez, M., Temple, S., Ansari, S. A., D'Amico, A. & Agris, P. F. Attomole quantification and global profile of RNA modifications: Epitranscriptome of human neural stem cells. Nucleic Acids Res 44, e26 (2016), cryo-EM analysis (See e.g., Natchiar, S. K., Myasnikov, A. G., Kratzat, H., Hazemann, I. & Klaholz, B. P. Visualization of chemical modifications in the human 80S ribosome structure. Nature 551, 472-477 (2017) or epitranscriptome sequencing technologies (See e.g., Li, X., Xiong, X. & Yi, C. Epitranscriptome sequencing technologies: decoding RNA modifications. Nat Methods 14, 23-31 (2017), it will be easier to determine which particular RNAs contain modifications. It can be seen from the history of scientific literature that advances in purification tend to precede new discoveries (e.g. Dr. Meischer's isolation of DNA in 1868). It is envisioned that this approach will similarly facilitate new discoveries of RNA science.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

What is claimed:

1. A method of purifying a single ribonucleic acid (RNA) species, comprising:
isolating a DNA nanoswitch-target complex within a gel medium, wherein the DNA nanoswitch-target complex comprises a DNA nanoswitch and a target-of-interest;
digesting the DNA nanoswitch and the gel medium to form digested byproducts, and a free target-of-interest; and
isolating the free target-of-interest, wherein the free target-of-interest is a single RNA species.

2. The method of claim 1, wherein the free target-of-interest comprises or consist of ribonucleic acid.

3. The method of claim 2, wherein the ribonucleic acid is a messenger RNA (mRNA), a catalytic ribozyme, a self-splicing RNA, a gene regulating RNA, a microRNA, ribosomal RNA, or viral RNA.

4. The method of claim 1, wherein isolating a DNA nanoswitch-target complex within a gel medium comprises electrophoresing the DNA nanoswitch-target complex in an electrophoresis gel and excising the DNA nanoswitch-target complex.

5. The method of claim 1, wherein the DNA nanoswitch is characterized as looped.

6. The method of claim 1, wherein digesting the DNA nanoswitch and the gel medium comprises contacting the DNA nanoswitch and the gel medium with a nuclease.

7. The method of claim 1, wherein the method further comprises, prior to isolating a DNA nanoswitch-target complex, contacting a preselected deoxyribonucleic acid (DNA) nanoswitch and a target to form a DNA nanoswitch-target complex.

8. The method of claim 1, wherein isolating comprises dissolving the digested byproducts and purifying the free target-of-interest.

9. The method of claim 1, wherein the digested byproducts comprise agarose gel pieces, enzyme, and nanoswitch fragments.

10. A method of purifying two or more single ribonucleic acid (RNA) species, comprising:
isolating at least a first DNA nanoswitch-target complex and a second DNA nanoswitch-target complex within a gel medium, wherein the first DNA nanoswitch-target complex comprises a first DNA nanoswitch and a first target-of-interest and the second DNA nanoswitch-target complex comprises a second DNA nanoswitch and a second target-of-interest;
digesting the first DNA nanoswitch, second DNA nanoswitch, and gel medium to form digested byproducts, a first free target-of-interest, and a second free target-of-interest; and
isolating the first free target-of-interest and the second free target-of-interest, wherein the first free target-of-interest and the second free target-of-interest are different single RNA species.

11. The method of claim 10, wherein the first free target-of-interest and second free target-of-interest comprise or consist of ribonucleic acid.

12. The method of claim 11, wherein the ribonucleic acid is messenger RNA (mRNA), catalytic ribozyme, self-splicing RNA, or a gene regulating RNA.

13. The method of claim 10, wherein the first DNA nanoswitch and second DNA nanoswitch are each characterized as looped.

14. The method of claim 10, wherein isolating comprises electrophoresing the first and second nanoswitch-target complexes in an electrophoresis gel and excising the first and second nanoswitch-target complexes.

15. The method of claim 10, wherein digesting comprises contacting the first DNA nanoswitch, second DNA nanoswitch, and gel medium with DNase.

16. The method of claim 15, wherein the DNAse is DNase I.

17. The method of claim 10, wherein isolating comprises dissolving the digested byproducts and purifying the first free target-of-interest and the second free target-of-interest.

18. The method of claim 10, wherein the digested byproducts comprise agarose gel pieces, enzyme, and nanoswitch fragments.

19. A method of purifying a single ribonucleic acid (RNA) species, comprising:
contacting a deoxyribonucleic acid (DNA) nanoswitch and an RNA target to form a DNA nanoswitch-RNA target complex;
isolating the DNA nanoswitch-RNA target complex within a medium;
freeing the RNA target from the DNA nanoswitch-RNA target complex to form free RNA; and
isolating the free RNA, wherein the free RNA is a single RNA species.

20. The method of claim 19, wherein the free RNA is a messenger RNA (mRNA), a catalytic ribozyme, a self-splicing RNA, or a gene regulating RNA.
